(19) United States
(12) Patent Application Publication
(10) Pub. No.: US 2025/0213533 A1
(43) Pub. Date: Jul. 3, 2025

(54) 5-MEO-DMT FOR USE IN THE TREATMENT OF SLEEP DISTURBANCE

(71) Applicant: GH RESEARCH IRELAND LIMITED, Dublin 2 (IE)

(72) Inventors: Theis TERWEY, Berlin (DE); Conor BURKE, Laytown (IE); Naoise GAFFNEY, Dublin (IE)

(73) Assignee: GH RESEARCH IRELAND LIMITED, Dublin 2 (IE)

(21) Appl. No.: 18/851,349

(22) PCT Filed: Mar. 27, 2023
(86) PCT No.: PCT/EP2023/057828

§ 371 Date: Sep. 26, 2024

(30) Foreign Application Priority Data
Mar. 27, 2022 (EP) 22000084.8
Mar. 27, 2022 (EP) 22000085.5
Jan. 30, 2023 (EP) 23153996.6
Jan. 30, 2023 (EP) 23154014.7

(51) Int. Cl. A61K 31/4045; A61K 9/00; A61P 25/24

(52) U.S. Cl. CPC A61K 31/4045; A61K 9/0073; A61P 25/24

(57) ABSTRACT

5-Methoxy-N,N-dimethyltryptamine (5-MeO-DMT) or a pharmaceutically acceptable salt thereof is used in treating a patient suffering from sleep disturbance, wherein the 5-MeO-DMT or a pharmaceutically acceptable salt thereof is administered via the intravenous, intramuscular or subcutaneous route.

### TECHNICAL FIELD

The present invention is directed to improved methods for the treatment of sleep disturbance, in particular sleep disturbance in a patient suffering from a mental disorder or a nervous system disorder, such as a disorder characterized by depressive episodes, for example, Major Depressive Disorder (MDD), Postpartum Depression (PPD), Persistent Depressive Disorder, Seasonal Affective Disorder and Bipolar Disorder (BD), such as Bipolar I Disorder and Bipolar II Disorder; Anxiety Disorder, for example, Separation Anxiety Disorder, Agoraphobia, Generalised Anxiety Disorder (GAD), Social Anxiety Disorder (SAD), Panic Disorder, Phobia, and Substance/Medication Induced Anxiety Disorder; Somatic Symptom Disorder; Obsessive compulsive and Related Disorders, for example, Obsessive Compulsive Disorder (OCD) and Body Dysmorphic Disorder (BDD); Post-Traumatic Stress Disorder (PTSD); Pain Disorders, for example Chronic Pain, Fibromyalgia, and Migraine; Mental and Behavioural Disorders due to Psychoactive Substance Use, for example, Substance Use Disorder (SUD); Psychotic Disorders, for example Schizophrenia; Huntington's Disease; Parkinson's Disease; Dementia, for example Alzheimer's Dementia (AD), Parkinson's Disease Dementia, Dementia with Lewy Bodies, Vascular Dementia, Fronto-Temporal Dementia; Eating Disorders; Attention Deficit Hyperactivity Disorder (ADHD); Personality Disorders, for example Schizotypal Personality Disorder and Borderline Personality Disorder; Autism Spectrum Disorder; Chronic Fatigue Syndrome; a mental disorder or a nervous system disorder associated with HIV, Traumatic Brain Injury or Post COVID Condition.

The treatment comprises administering to a patient in need thereof a therapeutically effective amount of 5-methoxy-N,N-dimethyltryptamine (5-MeO-DMT) or of a pharmaceutically acceptable salt thereof.

### BACKGROUND OF THE INVENTION

Sleep disturbance (sleep dysregulation) refers to conditions that affect sleep quality, timing, or duration. It impacts a person's ability to properly function while the person is awake.

Sleep disturbances can be either idiopathic or can occur in the context of a medical condition such as for example mental disorders or nervous system disorders. In fact, several mental disorders and nervous system disorders are known to be associated with sleep disturbance.

Sleep disturbance can not only have a severe impact on the quality of life but can also lead to various secondary health problems.

Thus, there is a need for a treatment of sleep disturbance, in particular sleep disturbance associated with a mental disorder or a nervous system disorder.

### SUMMARY OF THE INVENTION

An aim of the invention is in particular the provision of therapies which are more effective (i.e., a) a larger percentage of patients experiencing a clinical response, b) a larger average clinical response, c) an earlier onset of the clinical response, and/or d) a more durable clinical response) than previously described therapies.

A further aim of the current invention is to provide a compound for improved psychoactive therapies and dosing regimens for said therapies which have a better safety profile and/or are better tolerated than previously described therapies. Another aim of the current invention is to provide a compound for improved psychoactive therapies and dosing regimens for said therapies which are more convenient than previously described therapies. Another aim of the current invention is to provide a compound for improved psychoactive therapies and dosing regimens for said therapies which are associated with higher rates of patient compliance (including higher rates of treatment initiation) than previously described therapies. A still further aim of the current invention is to identify specific disease aspects and specific subgroups of disease aspects which benefit from such improved psychoactive therapies.

The present invention provides 5-Methoxy-N,N-dimethyltryptamine (5-MeO-DMT) or a pharmaceutically acceptable salt thereof for use in treating a patient suffering from sleep disturbance, in particular insomnia, hypersomnia and/or a circadian rhythm disorder.

The invention also relates to 5-MeO-DMT or a pharmaceutically acceptable salt thereof for use in treating a patient suffering from sleep disturbance such as parasomnia, a sleep-related breathing disorder or a sleep-related movement disorder.

The sleep disturbance may be idiopathic or occur in a patient suffering from a mental disorder or a nervous system disorder, such as a disorder characterized by depressive episodes, for example, Major Depressive Disorder (MDD), Postpartum Depression (PPD), Persistent Depressive Disorder, Seasonal Affective Disorder and Bipolar Disorder (BD), such as Bipolar I Disorder and Bipolar II Disorder; Anxiety Disorder, for example, Separation Anxiety Disorder, Agoraphobia, Generalised Anxiety Disorder (GAD), Social Anxiety Disorder (SAD), Panic Disorder, Phobia, and Substance/Medication Induced Anxiety Disorder; Somatic Symptom Disorder; Obsessive compulsive and Related Disorders, for example, Obsessive Compulsive Disorder (OCD) and Body Dysmorphic Disorder (BDD); Post-Traumatic Stress Disorder (PTSD); Pain Disorders, for example Chronic Pain, Fibromyalgia, and Migraine; Mental and Behavioural Disorders due to Psychoactive Substance Use, for example, Substance Use Disorder (SUD); Psychotic Disorders, for example Schizophrenia; Huntington's Disease; Parkinson's Disease; Dementia, for example Alzheimer's Dementia (AD), Parkinson's Disease Dementia, Dementia with Lewy Bodies, Vascular Dementia, Fronto-Temporal Dementia; Eating Disorders; Attention Deficit Hyperactivity Disorder (ADHD); Personality Disorders, for example Schizotypal Personality Disorder and Borderline Personality Disorder; Autism Spectrum Disorder; Chronic Fatigue Syndrome; a mental disorder or a nervous system disorder associated with HIV, Traumatic Brain Injury or Post COVID Condition.

The present invention also provides dose ranges and dosing regimen useful for the treatment of sleep disturbance.

In the context of the present invention, 5-MeO-DMT or a pharmaceutically acceptable salt thereof is administered via intravenous, intramuscular or subcutaneous administration.

5-MeO-DMT or a pharmaceutically acceptable salt thereof is administered at a dose or in a dosage regimen that causes the patient to experience a peak psychedelic experience. A dosage of about 1 mg to about 10 mg 5-MeO-DMT or an equimolar amount of a pharmaceutically acceptable salt may be administered.

### DETAILED DESCRIPTION OF THE INVENTION

### Definitions

As used in the context of the present invention, unless otherwise noted, the term “5-MeO-DMT” refers to the free base 5-MeO-DMT. It is contemplated that pharmaceutically acceptable salts of 5-MeO-DMT may also be used. Such salts are in particular acid addition salts, wherein the acid may be selected from, for instance, acetic acid, benzoic acid, citric acid, fumaric acid, hydrobromic acid, hydrochloric acid, hydrofluoric acid, hydroiodic acid, oxalic acid, succinic acid and triflic acid. A preferred example is the hydrobromide salt. The appropriate weight amount of a salt to be administered can be calculated from the weight amount of the free base, assuming that equimolar amounts are used.

As used in the context of the present invention, a “patient” to be treated is a human subject who is suffering from sleep disturbance by a licensed professional in accordance with accepted medical practice or who is diagnosed by a licensed professional in accordance with accepted medical practice with a mental disorder or a nervous system disorder associated with sleep disturbance. In the latter case, assessing sleep disturbance may or may not be part of the diagnosis.

Diagnosis of a mental disorder or a nervous system disorder can, for instance, be in accordance with the Diagnostic and Statistical Manual of Mental Disorders—Fifth Edition (DSM-5) published by the American Psychiatric Association. In some instances, as is apparent from the discussion of specific conditions below, the criteria may be modified or supplemented to better define patients or patient groups particularly benefiting from a treatment according to the invention. The diagnosis will in any event be by a physician or a psychologist. It is not sufficient that the human subject himself/herself considers that he/she is suffering from the disorder.

As used in the context of the present invention, unless otherwise noted, the terms “treating” and “treatment” shall include the management and care of a patient for the purpose of combating a disease, condition, or disorder and includes the administration of compounds and methods according to the present invention to alleviate the signs and/or symptoms of the disease or eliminate the disease, condition, or disorder.

“Treatment of sleep disturbance” shall include the management and care of a patient for the purpose of combating sleep disturbance and includes the administration of compounds and methods according to the present invention to alleviate the signs and/or symptoms of sleep disturbance or eliminate sleep disturbance. The sleep disturbance may be idiopathic or may be associated with a mental disorder or a nervous system disorder.

The patient may suffer from treatment resistant disease. Treatment resistance means that the patient had no adequate improvement after at least two adequate courses of therapy. The patient in particular had no adequate improvement after at least two adequate courses of therapy, wherein at least one of the two courses was a pharmacotherapy; for instance, the patient had no adequate improvement after at least two adequate courses of pharmacotherapy. The at least two prior courses of treatment were in particular administered in the current episode of the disease, for instance, if the patient suffers from a disorder characterized by depressive episodes, in the current episode of depression.

As used in the context of the present invention, unless otherwise noted, the term “therapeutically effective amount” shall mean the amount of active compound or pharmaceutical ingredient that elicits the biological or clinical response in a human that is being sought by a researcher, medical doctor or other clinician, which includes alleviation of the signs and/or symptoms of the disease, condition or disorder being treated.

“Clinical response” includes, but is not limited to, improvements on rating scales. These scales assess (i) sleep disturbance or aspects of sleep disturbance and/or (ii) a mental disorder or nervous system disorder or aspects of such a disorder.

The severity of a condition as well as changes of the severity can be assessed by the Clinical Global Impression (CGI) rating scales which are measures of symptom severity, treatment response and the efficacy of treatments.

The CGI rating scales were developed to provide a brief, stand-alone assessment of the clinician's view of the patient's global functioning prior to and after a treatment (Busner, J. and Tagrum, S. D., 2007. The Clinical Global Impressions Scale: Applying a Research Tool in Clinical Practice. Psychiatry 2007, 29-37).

The CGI-Severity (CGI-S) is based on one question the clinician has to answer: “Considering your total clinical experience with this particular population, how mentally ill is the patient at this time?” This is rated on the following seven-point scale: 1=normal, not at all ill; 2=borderline mentally ill; 3=mildly ill; 4=moderately ill; 5=markedly ill; 6=severely ill; 7=among the most extremely ill patients.

The CGI-S can be used to assess treatment success by comparing scores before and after treatment.

Alternatively, treatment success can be assessed using the CGI-Improvement (CGI-1), which is similarly simple in its format. After the treatment, the clinician compares the patient's overall clinical condition to the one prior to the treatment (the so-called baseline value). Again, only one query is rated on a seven-point scale: “Compared to the patient's condition at admission to the project [prior to medication initiation], this patient's condition is: 1=very much improved since the initiation of treatment; 2=much improved; 3=minimally improved; 4=no change from baseline (the initiation of treatment); 5=minimally worse; 6=much worse; 7=very much worse since the initiation of treatment.”

The Patient Global Impression scale (PGI), also known as Subject Global Impression (SGI), is the counterpart to the Clinical Global Impressions scale (CGI). It consists of one item based on the CGI and adapted to the patient. It can measure disease severity (PGIS) or disease improvement (PGI-I).

Individual items of scales as described herein as well as sub-combinations of individual items may be used to assess specific disease aspects.

As used in the context of the present invention, unless otherwise noted, the term “administration” (or “application”) shall mean the introduction of an amount, which may be a predetermined amount, of active compound or pharmaceutical ingredient into a patient via any route. The active compound is administered by intravenous administration, by intramuscular administration or by subcutaneous administration.

As used in the context of the present invention, unless otherwise noted, the terms “dose” and “dosage” and “dosage amount” shall mean the amount of active compound or pharmaceutical ingredient which is administered to a patient in an individual administration. The term “dosage regimen” (or “dosing regimen”) shall mean a defined sequence of one or more individual administrations.

### Sleep Disturbance

There are two fundamental types of sleep: rapid eye movement (REM) sleep and non-REM sleep. Non-REM sleep can be divided into four stages (I-IV). These non-REM stages correspond to an increasing depth of sleep. Non-REM and REM sleep alternate during each of the four to five cycles of normal human sleep each night. During the earlier proportion of the night, non-REM sleep is deeper and occupies a disproportionately large amount of time, particularly within the first cycle of sleep. As the night progresses, non-REM sleep becomes shallow and more of each cycle is allocated to REM sleep.

Normal healthy sleep consists of different phases as outlined above that proceed in successive, tightly regulated order through the night.

Disruption of this tight regulation results in sleep disturbances.

Sleep disturbance refers to conditions, whether idiopathic or occurring in the context of a medical condition such as for example a mental disorder or a nervous system disorder, that affect sleep quality, timing, or duration. It impacts a person's ability to properly function while the person is awake.

Common forms of sleep disturbances encompass disorders of initiating and maintaining sleep (insomnia), disorders of excessive somnolence (hypersomnia), disorders of sleep-wake schedule (circadian rhythm disorders), dysfunctions associated with sleep, sleep stages, or partial arousals (parasomnia), disorders characterized by respiratory disturbance during sleep (sleep-related breathing disorders) and disorders characterized by abnormal movements during sleep (sleep-related movement disorders).

Insomnia is a sleep disturbance where people have difficulty falling or staying asleep. People with insomnia have difficulty falling asleep; wake up often during the night and have trouble going back to sleep; wake up too early in the morning; have unrefreshing sleep; and/or have at least one daytime problem such as fatigue, sleepiness, problems with mood, concentration, accidents at work or while driving, etc. due to poor sleep.

Hypersomnia is characterized by excessive daytime sleepiness, and/or prolonged nighttime sleep. Sleep drunkenness is also a symptom found in hypersomnia patients. It is a difficulty transitioning from sleep to wake. Individuals experiencing sleep drunkenness report waking with confusion, disorientation, slowness and repeated returns to sleep.

Circadian rhythm disorders are characterized by chronic or recurring sleep disturbances due to alterations of the individual's internal circadian rhythm or due to misalignments between their circadian rhythm and their desired or required work or social schedule. This dyssynchrony may be transient or persistent. The ensuing clinical picture combines elements of both insomnia and hypersomnia. Sleep periods are usually shortened and disrupted, performance during the desired waking state is impaired, and temporary opportunities to revert to a regular sleep schedule are unsuccessful.

Parasomnia designates various forms of sleep disturbance characterized by abnormal behavioural or physiological activity (such as sleepwalking or nightmares) that people experience prior to falling asleep, while asleep, or during the arousal period between sleep and wakefulness. There are considerable variations in terms of characteristics, severity, and frequency. Parasomnia may compromise the quality of sleep.

Sleep-related breathing disorders are characterized by abnormal and difficult respiration during sleep. Respiration is a complex process that relies heavily on the coordinated action of the muscles of respiration and the (control center in the) brain. One form of a sleep-related breathing disorder is central sleep apnoea. It occurs when the brain stops sending signals that control breathing, for instance, based on an underlying health condition. Central sleep apnoea has a potentially serious impact on sleep and the balance of oxygen and carbon dioxide in the blood. The reduction of airflow leads to intermittent hypoxia which in leads to sleep fragmentation due to microarousals or awakenings. A consequence can be excessive daytime sleepiness.

In sleep-related movement disorders repetitive, relatively simple, usually stereotyped, movements interfere with sleep or its onset. The most common of these are restless leg syndrome (RLS) and periodic limb movement disorder (PLMD).

Not getting the proper amount or quality of sleep may lead to personality changes and may not only exacerbate existing mental illness, but also be a trigger for the development of mental illness. Sleep disturbance may also interfere with cognitive function and lead to memory impairment. A subject who is deprived of sleep may experience difficulty making decisions, irritability, have problems with performance, and may have slower reaction times. Sleep loss can also adversely affect life by contributing to the development of obesity, diabetes, and heart disease.

Treatment of sleep disorders varies depending on the type and underlying cause. Maintenance of good sleep hygiene, a healthy sleep environment, and a consistent sleep-wake schedule are often considered as first-line treatment. If not successful, treatment also involves pharmacotherapy or psychotherapy.

Available treatments are not successful in all patients, may be associated with side effects and/or require treatment over a long period of time to achieve a relevant treatment effect.

In patients suffering from sleep disturbance in association with a mental disorder or a nervous system disorder known treatments of the mental or nervous system disorder do not necessarily improve the sleep disturbance.

For instance, sleep disturbance is frequently associated with mental disorders, such as depression. However, treatment of depression does not necessarily lead to an improvement of the concomitant sleep disturbances. While most antidepressants have been proven to influence the sleep architecture, some classes of antidepressants improve sleep, but others may cause sleep impairment.

### Measuring Sleep Disturbance

Sleep can be assessed by measuring parameters such as sleep duration, sleep architecture, sleep latency, and the frequency and duration of awakenings throughout the night. The quantitative metrics may be measured using objective methods, including polysomnography, actigraphy, and the determination of sleep latency, or by way of self-reported measures (questionnaires).

Polysomnography is a technique requiring that a patient is monitored overnight at a specialized clinic. A variety of functions are measured throughout the night, including eye movements, brain and muscle activity, respiratory effort and airflow, blood oxygen levels, body positioning and movements, snoring, and heart rate.

Another quantitative measurement is actigraphy. An actimetry sensor is worn to measure motor activity, which is recorded continually and used to assess sleep-wake cycles. This technique allows the patient to continue normal routines while the required data are being recorded in natural sleep environment.

Sleep latency can be measured by the multiple sleep latency test (MSLT). This test provides an objective measure to determine how long it takes a person to fall asleep across a multiplicity of test naps. An average sleep latency of approximately 10 minutes is considered to be normal; less than eight minutes is indicative of sleep disturbance (excessive daytime sleepiness). Accompanying analysis of brain activity can assist in the further diagnosis of the sleep disturbance.

Sleep-rating questionnaires capture ratings of components of sleep quality, such as perceptions of sleep depth, rousing difficulties, and restfulness after sleep, in addition to other factors that could affect sleep quality, such as comorbid conditions and medication use. The evaluation of the qualitative aspects of sleep experience is important, as sleep complaints can often persist despite normal values for quantitative measures of sleep.

Questionnaires not only facilitate a quick and accurate assessment of a complex clinical problem, but they are potentially also helpful for tracking a patient's progress.

Various sleep quality indexes are known. The following indexes include examples of questionnaires to assess sleep in general and questionnaires to assess in particular insomnia, hypersomnia, circadian rhythm disorders and parasomnia, respectively. The invention is, however, not limited to the use of a particular index or questionnaire.

Some questionnaires rely on recall periods (recall windows) of several days or even weeks. While this may be appropriate for diagnosing sleep disturbances, it is not always appropriate for assessing treatment effects, in particular rapid onset of effect after treatment. For several of the questionnaires the recall period can be modified so that the scores obtained reflect a period after treatment. Questionnaires specifically discussed herein to assess effects of a treatment on sleep in patients suffering from specific conditions rely on a recall period that does not start earlier than the time point when acute psychedelic experiences have subsided after the last administration. To meet this criterion, the normally applied recall period is modified if necessary.

Sleep quality in general can be assessed, for instance, with the Sleep-50 questionnaire.

The SLEEP-50 questionnaire consists of 50 items designed to screen for a variety of sleep disorders in the general population. The scale consists of nine subscales, reflecting some of the most common disorders and complaints related to sleep and the factors required for diagnosis such as sleep apnoea, insomnia, narcolepsy, restless legs/periodic leg movement disorder, circadian rhythm sleep disorder, sleepwalking, nightmares, factors influencing sleep, and the impact of sleep complaints on daily functioning. For each item, respondents are provided with a scale ranging from 1 (“not at all”) to 4 (“very much”) and are asked to indicate the extent to which the statement has matched their experience over the previous month or another appropriate recall window.

For diagnosing a sleep disorder not only the specific subscale (e.g., insomnia) must exceed a certain cut-off point, but respondents must also meet a cut-off of at least 3 or 4 (“rather much” or “very much”, respectively) on the subscale evaluating the impact of sleep complaints on daily functioning (Spoormaker et al., Initial validation of the SLEEP-50 questionnaire. Behav Sleep Med. 2005; 3(4):227-46).

Treatment success is indicated (i) by a decrease of the score, preferably (ii) by a decrease to below the cut-off value.

A common questionnaire assessing sleep disturbance is the Pittsburgh Sleep Quality Index. Other instruments are the insomnia severity index, the Espie sleep disturbance questionnaire and the Patient Reported Outcomes Measurement Information System (PROMIS®) Sleep Disturbance.

The Pittsburgh Sleep Quality Index (PSQI) assesses overall sleep quality and disturbances. The PSQI is a self-rated questionnaire comprising 19 questions. Respondents are asked to indicate how frequently they have experienced certain sleep difficulties over the past month or another appropriate recall window.

The 19 self-rated questions assess a wide variety of factors relating to sleep quality, including estimates of sleep duration and latency and of the frequency and severity of specific sleep-related problems. These 19 items are grouped into seven component scores: (1) subjective sleep quality; (2) sleep latency; (3) sleep duration; (4) habitual sleep efficiency; (5) sleep disturbances; (6) use of sleeping medication; (7) daytime dysfunction.

Each component is assigned a score of 0 to 3. Higher scores indicate more acute sleep disturbances. Detailed Scoring Instructions for the Pittsburgh Sleep Quality Index can be found in the Appendix of Buysse et al. The Pittsburgh Sleep Quality Index: a new instrument for psychiatric practice and research. Psychiatry Res. 1989 May; 28(2):193-213.

The seven component scores are then summed to yield one global score, with a range of 0-21 points, “0” indicating no difficulty and “21” indicating severe difficulties in all areas. A global score cut-off of 5 distinguishes poor from good sleepers. A global score >5 indicates that a patient is having severe difficulties in at least two areas, or moderate difficulties in more than three areas.

If treatment outcome is assessed using the PSQI, treatment success is indicated (i) by a decrease of the score, preferably (ii) by a decrease to 5 or below.

The insomnia severity index (ISI) is a short questionnaire relating to subjective sleep quality, severity of symptoms, subjective satisfaction with sleep, the degree to which insomnia interferes with daily functioning, how noticeable the respondent feels his or her insomnia is compared to others, and the overall level of distress created by the sleep problem. Individual responses can be scored from 0 (=none) to 4 (=very); a higher total score corresponds to more severe insomnia. A total score of 0-7 indicates “no clinically significant insomnia,” 8-14 means “subthreshold insomnia,” 15-21 is “clinical insomnia (moderate severity),” and 22-28 means “clinical insomnia (severe)” (A. Shahid et al. (eds.), STOP, THAT and One Hundred Other Sleep Scales, Springer Science+Business Media, LLC 2012. The recall window is two weeks. Another appropriate recall window can also be used.

Treatment success is indicated (i) by a decrease of the score, for instance, by >7 points, in particular >8 point; preferably (ii) by a decrease to below the cut-off value for clinically significant insomnia.

The Espie sleep disturbance questionnaire (SDQ) evaluates subjective experiences of insomnia. With ratings on restlessness/agitation, mental overactivity, consequences of insomnia, and lack of sleep readiness, the SDQ is concerned specifically with beliefs about the sources of sleep issues. Respondents use a five-point scale to indicate how often certain statements about insomnia are representative of their experience. 1 means “never true,” while 5 means “very often true.” Higher scores are indicative of more dysfunctional beliefs about the causes and correlates of insomnia (A. Shahid et al., loc. cit.).

Treatment success is indicated by a decrease of the score.

The Patient-Reported Outcomes Information System (PROMIS)® Sleep Disturbance instrument is a universal measure to evaluate sleep disturbances. The instrument is available as a long form and 4 different short forms (e.g., 4-, 6-, and 8-items) and assesses self-reported perceptions of sleep quality, sleep depth, and any perceived difficulties related to getting and staying asleep over a 7-day period.

Each item on the measure is rated on a 5-point scale. The raw scores on the items are summed to obtain a total raw score. Total raw scores are then converted into a standardized T-score using conversion tables.

Treatment success is indicated by a decrease of the T-score.

Hypersomnia or hypersomnolence can be assessed by the Epworth Sleepiness Scale, the Stanford Sleepiness Scale, or the Idiopathic Hypersomnia Severity scale.

The Epworth Sleepiness Scale (ESS) evaluates overall daytime sleepiness. The questionnaire asks respondents to rate how likely they are to fall asleep in eight different situations representing a moment of relative inactivity, such as a nap in the afternoon or sitting in a car stopped in traffic. Using a scale of 0-3 (with 0 meaning “would never doze” and 3 meaning “high chance of dozing”), respondents rate their likelihood of falling asleep. Scoring ranges from 0-24; the higher the score, the higher the severity of daytime sleepiness. A cut-off score of 10 identifies daytime sleepiness at a potentially clinical level (A. Shahid et al., loc. cit.).

Treatment success is indicated (i) by a decrease of the score, preferably (ii) by a decrease to 10 or below.

The Stanford Sleepiness Scale is a subjective measure of sleepiness, evaluating sleepiness at specific moments in time. Consisting of only one item, the scale requires respondents to select one of seven statements best representing their current level of perceived sleepiness. A scale from 1 (=Feeling active and vital; alert; wide awake) to 7 (=Almost in reverie; sleep onset soon; lost struggle to remain awake) is used to assess the level of sleepiness (A. Shahid et al., loc. cit.).

Treatment success is indicated by a decrease of the score.

Parasomnias can be evaluated by the Paris Arousal Disorders Severity Scale (PADSS).

The Paris Arousal Disorders Severity Scale (PADSS) is a self-rating scale listing parasomniac behaviours, assessing their frequency and includes an evaluation of consequences (Amulf et al., A scale for assessing the severity of arousal disorders. Sleep. 2014 Jan. 1; 37(1):127-36).

Treatment success is indicated (i) by a decrease of the score, preferably (ii) by a decrease to below the cut-off value.

A common questionnaire that assesses sleep-related breathing disorders is the Berlin Questionnaire (A. Shahid et al., loc. cit.). An appropriate recall period can also be chosen.

Treatment success is indicated by a decrease of the score.

A common questionnaire that assesses sleep-related movement disorders is the International Restless Legs Syndrome Study Group Rating Scale. The 10-item questionnaire asks respondents to use Likert-type ratings to indicate how acutely the disorder has affected them over the course of the past week. Questions can be divided into one of two categories: disorder symptoms (nature, intensity, and frequency) and their impact (sleep issues, disturbances in daily functioning, and resultant changes in mood. Each of the ten questions requires respondents to rate their experiences with RLS on a scale from 0 to 4, with 4 representing the most severe and frequent symptoms and 0 representing the least. Total scores can range from 0 to 40. As a brief scale with excellent psychometric qualities, the instrument may be suitable for a variety of research and clinical purposes, including screening and assessment of treatment outcomes. (A. Shahid et al., loc. cit.).

Treatment response can be assessed by a decrease of the score.

### Scales to Assess Mental and Nervous System Disorders

Numerous scales have been suggested to assess severity of mental disorders or nervous system disorders. Such scales are based on tests which can be self-administered or administered by a clinician.

Scales for the assessment of mental or nervous system disorders which may be used according to the invention include those known in the art for diagnosis and/or monitoring the mental or nervous system disorders discussed in more detail below.

Treatment outcome is assessed by using one or more indices or scales at one or more time points after completion of a treatment course.

The assessment can be carried out after the acute psychedelic experience has subsided. An appropriate point in time for an early assessment is generally about 2 to 3 hours after the last administration. An early assessment can generally be carried out, for instance, about 2 hours or about 3 hours after the last administration.

An assessment of an effect on sleep disturbance or an effect on a mental or nervous system disorder related to an effect on sleep disturbance can, however, be carried out at the earliest on the day after the treatment (i.e., on day 1) so that the treated patient had the opportunity to sleep for at least one night.

Thus, an assessment at day 1 or on day 1 means an assessment on the day following the administration. The assessment will be carried out not earlier than 12 hours after the last administration and in any event not earlier than one night after the last administration and not later than 36 hours after the last administration. The assessment can be carried out after about 24 hours.

An assessment at day 7 or on day 7 means an assessment on the seventh day following the administration (the day of administration is day 0). Analogous definitions apply for other assessment timings measured in days.

When assessing a clinical response, for instance, using one of the scales to assess severity of a mental disorder or a nervous system disorder, at an early timepoint after drug administration (e.g. at 2 hours) based on endpoints which have been developed for a longer recall period (e.g. normally 7 days for the MADRS), a rational modification of such endpoint (e.g. changing the MADRS recall period to 2 hours and carrying forward the sleep item recorded at baseline before drug administration) may be applied. The same applies with respect to any other scale applied herein to assess treatment effects on a mental or nervous system disorder, unless a recall period is specifically indicated.

The considerations outlined apply for early timepoints because, on the one hand, in order to assess a clinical response, the influence of the patient's status before the treatment on any score recorded after treatment should be kept as low as possible, whereas on the other hand the sleep item cannot be assessed 2 hours after drug administration.

At later timepoints, for instance, on day 1 or later, typically all items of the relevant scales to assess a clinical response can be assessed, using, if required, an adapted recall period, so that it is not necessary to carry forward any pre-treatment score.

### Resting State Networks and Sleep Disturbance

Brain processes can be studied by functional magnetic resonance imaging (fMRI). Brain activity is associated with blood flow, and temporal correlations of spontaneous blood oxygen level dependent (BOLD) signal fluctuations between different brain areas can be measured.

Functional images of the brain are acquired over the course of several minutes. Patterns of low-frequency BOLD signal oscillation are observed across the brain. The decomposition of this spontaneous signal reveals distributed areas with correlated and anti-correlated fluctuations.

In this way, resting-state fMRI can be used to characterize large-scale functional networks, so-called resting-state networks (RSN), which are a set of spatially distinct brain regions that show coordinated activity in the absence of any explicit cognitive task (i.e., at rest). The observed patterns, characterizing a network of brain regions with coherent patterns of signal variation, are called resting-state networks (RSN).

Different resting state networks have been identified and named mostly based on spatial similarity between the resting state networks and activation patterns seen in task fMRI experiments.

Resting-state fMRI can therefore be used to assess the intrinsic functional organization of the brain. Resting-state networks have been characterized for aspects of attention, memory, cognitive control, default mode, motor, and sensory system.

RSNs have been shown to be responsible for various aspects of complex brain function, and it has been found that these connectivity networks are compromised in various disease states. Such disease states are associated with altered functional connectivity within a specific resting state network and/or between one or more regions in one or more additional resting state networks.

Altered resting state networks can be found in insomnia, hypersomnia, circadian rhythm disorders, parasomnias, sleep-related breathing disorders and sleep-related movement disorders.

A key network involved in sleep is the default mode network (DMN). Generally, the DMN is deactivated during tasks and activated at rest. It is involved in multiple cognitive processes such as higher cognition, emotion, and interoception. During sleep, its overall activity level decreases. Given the importance of the DMN for sleep physiology, altered activity of the DMN is of particular relevance in the context of sleep disturbance.

Compromised resting state networks can also be found in mental disorders or nervous system disorders, in particular a disorder characterized by depressive episodes, for example, Major Depressive Disorder (MDD), Postpartum Depression (PPD), Persistent Depressive Disorder, Seasonal Affective Disorder and Bipolar Disorder (BD), such as Bipolar I Disorder and Bipolar II Disorder; Anxiety Disorder, for example, Separation Anxiety Disorder, Agoraphobia, Generalised Anxiety Disorder (GAD), Social Anxiety Disorder (SAD), Panic Disorder, Phobia, and Substance/Medication Induced Anxiety Disorder; Somatic Symptom Disorder; Obsessive compulsive and Related Disorders, for example, Obsessive Compulsive Disorder (OCD) and Body Dysmorphic Disorder (BDD); Post-Traumatic Stress Disorder (PTSD); Pain Disorders, for example Chronic Pain, Fibromyalgia, and Migraine; Mental and Behavioural Disorders due to Psychoactive Substance Use, for example, Substance Use Disorder (SUD); Psychotic Disorders, for example Schizophrenia; Huntington's Disease; Parkinson's Disease; Dementia, for example Alzheimer's Dementia (AD), Parkinson's Disease Dementia, Dementia with Lewy Bodies, Vascular Dementia, Fronto-Temporal Dementia; Eating Disorders; Attention Deficit Hyperactivity Disorder (ADHD); Personality Disorders, for example Schizotypal Personality Disorder and Borderline Personality Disorder; Autism Spectrum Disorder; Chronic Fatigue Syndrome; a mental disorder or a nervous system disorder associated with HIV, Traumatic Brain Injury or Post COVID Condition.

Resting state networks involved in sleep disturbance are also affected by mental or nervous system conditions which are a consequence of certain medical health conditions.

In insomnia patients, dysfunctional connectivity is observed within the default mode network (DMN) and within the salience network, which is implicated in the detection and integration of emotional and sensory stimuli. Studies have suggested that these networks contain critical regions integrating emotional and bodily states, and the dysfunctional connectivity within and/or between these networks and other brain areas may underlie the vigilance, subjective distress, and poor sleep continuity of patients.

In hypersomnia patients, the default mode network is affected. For instance, in idiopathic hypersomnia, distinct DMN hubs—the precuneus and medial prefrontal cortex—demonstrate significant changes, and functional connectivity in the DMN correlates with self-reported sleepiness severity.

A study investigating differences between night shift nurses and day work nurses revealed that dysrhythmia of circadian rhythms contributes to resting-state functional changes in the cerebellum, involved in sleep regulation, and cognitive functions such as responsiveness and alertness. Moreover, the functional connectivity of the DMN is fundamentally different in early and late circadian phenotypes. Like other forms of sleep disturbance, circadian rhythm disorders can lead to changes in brain functional connectivity. Changes in resting state brain functional connectivity have been reported in various diseases with circadian rhythm disorders.

While functional brain imaging is technically difficult to perform during a parasomnia event, differences in the precuneus have been observed in disorders of arousal representing a non-REM parasomnia.

The precuneus is involved in the analysis and integration of visual, audio, and somesthetic information and the monitoring of movements. The precuneus is a subregion of the DMN. Thus, in patients with parasomnias the default mode network is affected.

Resting-state fMRI studies in patients suffering from sleep-related breathing disorders, such as central sleep apnoea, indicate significant global and regional connectivity deficits, especially in the default mode network (DMN) and regions involved in the arousal and sensorimotor systems.

Sleep-related movement disorders, such as for example periodic limb movements during sleep are reflected by alterations in the prefrontal motor control pathway, a subregion of the default mode network. Also, activity in the cerebellum and thalamus, with additional activation in the red nuclei and brainstem can be observed.

In many instances, aberrant functional connectivity of resting state networks involved in sleep disturbance are also involved in the conditions listed above. Thus, according to the invention, influencing those networks by a therapy according to the invention will lead to an improvement of the sleep disturbance and, if the patient treated suffers from a mental disorder or a nervous system disorder, also of that disorder.

### The Active Agent

The above discussion shows that sleep disturbances as such present a significant disease burden and deserve appropriate treatment.

The inventors considered that a carefully chosen hallucinogen may lead to an improved treatment of important aspects of sleep disturbances and may lead to overall improvements of the condition.

One group of hallucinogens entails compounds which bind to the 5-hydroxytryptamine (5-HT) receptors, which are also referred to as serotonin receptors (described are 7 families 5-HT1 to 5-HT7 with several subtypes). Examples are lysergic acid diethylamide (LSD), psilocybin, and N,N-dimethyltryptamine (DMT). These serotonergic agents are often referred to as “psychedelics”, which emphasizes their predominant ability to induce qualitatively altered states of consciousness such as euphoria, trance, transcendence of time and space, spiritual experiences, dissolution of self-boundaries, or even near-death experiences, while other effects such as sedation, narcosis, or excessive stimulation are only minimal.

Chemically, serotonergic psychedelics are either phenylalkylamines or indoleamines, with the indoleamine class being divided into two subsets, ergolines and tryptamines, the latter being derived from tryptamine.

The various serotonergic psychedelics have different binding affinity and activation potency for various serotonin receptors, particularly 5-HT1A, 5-HT2A, and 5-HT2C, and their activity may also be modulated by interaction with other targets such as monoamine transporters and trace amine-associated receptors.

Recently published clinical studies which have used serotonergic psychedelic drugs such as LSD, psilocybin and DMT (using the shamanic brew Ayahuasca, which contains DMT) in certain mental disorders suggest that those compounds could provide an alternative to the currently available treatments for certain mental disorders. However, there are reports that these compounds can induce mania in patients suffering from depressive symptoms, and this may preclude their clinical use.

For instance, Lake et al. (Lake, C. R., Stirba, A. L., Kinneman, R. E. Jr, Carlson, B., Holloway, H. C., 1981. Mania associated with LSD ingestion. American Journal of Psychiatry. 138(11):1508-9) report about a patient who suffered a manic attack after ingesting LSD or an LSD analogue. The patient experienced acute symptoms of LSD intoxication, which resolved but were followed in about 3 weeks by a typical manic episode of psychotic magnitude. Hendin and Penn (Hendin, H. M., Penn, A. D., 2021. An episode of mania following self-reported ingestion of psilocybin mushrooms in a woman previously not diagnosed with bipolar disorder: A case report. Bipolar Disorders 23(4):1-3) report about an episode of mania following self-reported ingestion of psilocybin mushrooms. Szmulewicz et al. (Szmulewicz, A. G., Valerio, M. P., and Jose M Smith, J. M., 2015. Switch to mania after ayahuasca consumption in a man with bipolar disorder: a case report. International Journal of Bipolar Disorders (2015) 3:4) report on a switch to mania after consumption of ayahuasca, a DMT containing brew, in a man with bipolar disorder.

A further case report is found in Brown, T., Shao, W., Ayub, S., Chong, D., & Cornelius, C. (2017). A Physician's attempt to self-medicate bipolar depression with N, N-dimethyltryptamine (DMT). Journal of Psychoactive Drugs, 49(4), 294-296.

The inventors considered that in order to avoid the induction of mania or hypomania or at least reduce the risk of induction of mania or hypomania, the compound administered must be appropriately chosen and preferably is administered in a particular dosing regimen.

The inventors identified 5-methoxy-N,N-dimethyltryptamine (5-MeO-DMT) as a psychedelic of particular interest for use in therapy. 5-MeO-DMT has a distinct pharmacological profile which differs from that of other psychedelic compounds.

5-MeO-DMT is a potent, fast-acting, naturally occurring serotonin (5-HT) agonist, acting at both the 5-HT1A and the 5-HT2A receptor, with higher affinity for the 5-HT1A receptor subtype compared to other classical psychedelics.

Inhibition constants (Kᵢ values) as further detailed on the example section below for psilocin (the dephosphorylated from of psilocybin which is formed after uptake of psilocybin), DMT and 5-MeO-DMT are 48, 38 and 1.80 nM, respectively, at 5-HT1A receptors located in the hippocampus of post-mortem human brain. Thus, 5-MeO-DMT exhibits high affinity and psilocin and DMT exhibit moderate affinity for 5-HT1A receptors. Inhibition constants (Kᵢ values) for psilocin, DMT and 5-MeO-DMT are 37, 117 and 122 nM, respectively, at 5-HT2A receptors located in the frontal cortex of post-mortem human brain. Therefore, psilocin exhibits moderate/strong affinity and DMT and 5-MeO-DMT exhibit comparatively weak affinity for 5-HT2A receptors.

Relative to the other psychoactive compounds mentioned previously, 5-MeO-DMT displays an enhanced affinity for the 5-HT1A receptor, where it acts as a potent agonist. In the case of psilocin and DMT, there is an increased contribution of 5-HT2A binding, relative to 5-MeO-DMT, with the latter displaying the largest differential affinity for 5-HT1A over 5-HT2A of the three compounds. Therefore, 5-HT1A binding plays a much bigger role in the overall effect of 5-MeO-DMT relative to 5-HT2A binding compared to the other two compounds.

It has been reported that 5-HT1A agonism reduces impulsivity and aggression, whereas 5-HT2A agonism can result in short-term increases in these same traits. Furthermore, the dopamine system has been implicated in contributing to mania, with increased dopamine drive being linked to mania. LSD, psilocybin and DMT all display increased affinity for a variety of dopamine receptors relative to 5-MeO-DMT

Compared to other psychedelics, like LSD, psylocibin or DMT, 5-MeO-DMT can be administered to patients, preferably using dosing schemes as described herein, without a significant risk of inducing mania or hypomania in a patient suffering from a mental or nervous system disorder, including a disorder characterized by depressive episodes, for example, Major Depressive Disorder (MDD), Postpartum Depression (PPD), Persistent Depressive Disorder, Seasonal Affective Disorder and Bipolar Disorder (BD), such as Bipolar I Disorder and Bipolar II Disorder; a Psychotic Disorder, such as Schizophrenia; or a personality disorder, such as Schizotypal Personality Disorder. The patient suffering from such a mental or nervous system disorder, treated according to the invention, does not experience treatment-emergent mania or hypomania.

It is also noted that reports of treatment-emergent mania or hypomania related to psychoactive substance use seem to indicate large quantities of the respective compounds (e.g., DMT/ayahuasca, psilocybin, LSD) were used.

The inventors' approach of sequential up-titration of 5-MeO-DMT significantly reduces the risk of excessive dose administration with its potential for attendant adverse events.

Still further, the induction by antidepressants of isolated events of hypomania has been reported in patients suffering from treatment resistant depression (TRD) (Bader, Cynthia D., and David L. Dunner. “Antidepressant-induced hypomania in treatment-resistant depression.” •Journal of Psychiatric Practice•13.4 (2007): 233-237). However, the recently concluded clinical trial of 5-MeO-DMT in TRD patients showed no evidence of hypomania induction.

5-MeO-DMT can induce peak experiences, i.e., experiences characterized by an emotional perspective shift, which is described as “loss of ego” which often culminates in an overwhelming sense of “oneness with the universe”, more rapidly than other psychedelics and has a short duration of acute psychedelic effects (5 to 30 minutes after intravenous injection compared with several hours for e.g. oral psilocybin and oral LSD). These characteristics of 5-MeO-DMT are associated with an improved therapeutic profile which can be explained by specific alterations of Resting State Network (RSN) activity under 5-MeO-DMT treatment.

Furthermore, 5-MeO-DMT is a 5-HT7 receptor agonist showing high affinity towards the receptor. The inventors determined, using recombinant human 5-HT7 receptor, [³H]LSD as a radio ligand and serotonin to estimate non-specific binding, a Kᵢ of 2.3 nM. The 5-HT7 receptor has a role in neurogenesis, synaptogenesis and dendritic spine formation.

Another feature of 5-MeO-DMT is its short half-life.

5-MeO-DMT is mainly inactivated through a deamination pathway mediated by monoamine oxidase A, and it is O-demethylated by cytochrome P450 2D6 (CYP2D6) enzyme.

The inventors investigated pharmacokinetic properties of 5-MeO-DMT and observed rapid absorption and distribution of inhaled 5-MeO-DMT, with maximum concentrations and pharmacological effects observed during and immediately after dosing.

An analysis of the pharmacokinetic properties of 5-MeO-DMT after inhalation shows a very rapid decline of the plasma concentration. Already 10 minutes after administration, the concentration drops to 10% of Cmax or below; after 2 hours, it is 1% of Cmax or below; after 3 hours, 5-MeO-DMT is no longer detectable in the plasma. This applies over the whole dose range tested (6 mg, 12 mg, 18 mg). No accumulation is observed upon repeated administration within a time frame of 1 to 4 hours. Up titration as disclosed herein will not lead to accumulation and thus not to higher plasma concentrations, for instance, 10 minutes, 2 hours, or 3 hours after administration.

The properties of 5-MeO-DMT make the compound especially suitable for the treatment of sleep disturbance, in particular for patients suffering from a mental disorder or a nervous system disorder.

The properties of 5-MeO-DMT also allow specific dosage regimens, as discussed in more detail below.

According to the invention, isotopic variants of 5-MeO-DMT and pharmaceutically acceptable salts thereof can also be used. When reference is made to the use of 5-MeO-DMT or a pharmaceutically acceptable salt thereof, the use of isotopic variants is also contemplated.

These variants are in particular deuterated forms of 5-MeO-DMT and pharmaceutically acceptable salts of such forms.

Deuterated forms of 5-MeO-DMT are forms having a higher deuterium content than expected based on the natural abundance of this isotope.

Deuterated forms of 5-MeO-DMT are in particular forms wherein deuterium has been introduced at one or more defined hydrogen positions.

Examples of deuterated forms of 5-MeO-DMT include, without limitation, 1-deuterio-2-(5-methoxy-1H-indol-3-yl)-N,N-dimethylethanamine, 1,1-dideuterio-2-(5-methoxy-1H-indol-3-yl)-N,N-dimethylethanamine, 1,1,2,2-tetradeuterio-2-(5-methoxy-1H-indol-3-yl)-N,N-dimethylethanamine, and N,N-dimethyl-2-[5-(trideuteriomethoxy)-1H-indol-3-yl]ethanamine.

Further examples include forms of 5-MeO-DMT wherein deuterium has been introduced at one or more hydrogen positions of the N-bound methyl groups. Still further examples include forms of 5-MeO-DMT wherein one or more deuterium atoms replace hydrogen atoms of the indole ring system. It is moreover noted that combinations of the above substitution patterns are also contemplated.

Preparation methods for these compounds are known in the art.

According to the invention, mixtures of deuterated forms of 5-MeO-DMT, mixtures of one or more deuterated form with non-deuterated 5-MeO-DMT, pharmaceutically acceptable salts of deuterated forms of 5-MeO-DMT, mixture of such salts as well as mixtures of salts of deuterated and non-deuterated 5-MeO-DMT can also be used.

Further according to the invention, deuterated 5-MeO-DMT and salts of deuterated 5-MeO-DMT are used in amounts that are equimolar to the amounts of the corresponding non-deuterated forms.

According to the invention, prodrugs of 5-MeO-DMT and pharmaceutically acceptable salts of such prodrugs can also be used. Such prodrugs of 5-MeO-DMT can be metabolically converted to 5-MeO-DMT. Thus, when reference is made to the use of 5-MeO-DMT or a pharmaceutically acceptable salt thereof, this can be replaced by a 5-MeO-DMT prodrug or a salt thereof.

In suitable prodrugs, the hydrogen in position 1 of the indole moiety is substituted by an organic moiety which can be split off after administration.

Examples of suitable organic moieties are —C(O)OR¹, —C(O)R², —CH(R³)OR⁴, —C(O)OCH(R³)OC(O)R⁴, —C(O)OCH(R³)OC(O)OR⁴, —CH(R³)C(O)R⁴, —CH(R³)OC(O)R⁴, —CH(R³)OC(O)OR⁴, wherein each of R¹, R², R³, and R⁴ is independently hydrogen, alkyl, heteroalkyl, cycloalkyl, heterocyclylalkyl, aryl, or heteroaryl, wherein each alkyl, heteroalkyl, cycloalkyl, heterocyclylalkyl, aryl, and heteroaryl is independently substituted or unsubstituted.

Preferred examples of organic moieties are —CH(R³)OC(O)R⁴ and —C(O)OR¹, wherein R¹, R³, and R⁴ are defined as above.

Prodrugs, especially those of the above structure, can also be used on the form of pharmaceutically acceptable salts.

Specific examples of prodrugs are 5-MeO-DMT carboxy-isopropyl valinate, preferably in salt form, in particular as ditrifluoroacetate (1-(((S)-2-amino-3-methylbutanoyl)oxy)-2-methylpropyl 3-(2-(dimethylamino)ethyl)-5-methoxy-1H-indole-1-carboxylate di-trifluoroacetate) and 5-MeO-DMT methyl pivalate (3-(2-(dimethylamino)ethyl)-5-methoxy-1H-indol-1-yl)methyl pivalate).

Preparation methods for prodrugs as discussed herein are known in the art.

According to the invention, the Tₘₐₓ value of the metabolite 5-MeO-DMT as measured in male Sprague-Dawley (SD) rats following oral dosing of the prodrug at 10 mg/kg is preferably 1 hour or less, more preferably 0.7 hours or less and in particular 0.5 hours or less.

Further according to the invention, prodrugs of 5-MeO-DMT and salts of prodrugs of 5-MeO-DMT are used in amounts that are equimolar to the amounts of the corresponding non-prodrug forms.

### Modes of Administration

The therapeutically effective amount of 5-MeO-DMT is administered by intravenous administration, by intramuscular administration or by subcutaneous administration. Administration via these routes can assure a rapid onset of action. A most preferred route of administration is administration via the intravenous route, i.e. by intravenous injection.

5-MeO-DMT can be employed as a pharmaceutically acceptable salt, preferably the hydrobromide salt, or in the form of a formulation for administration via injection, examples of excipients and vehicles for such formulations being known in the art.

### Dosing Regimen

The present invention also provides dose ranges, particular doses as well as dosing regimens (administration schemes) and appropriate routes of administration.

The invention is in part based on the inventors' conclusion that the occurrence of a peak psychedelic experience during the acute phase after administration of 5-MeO-DMT or a pharmaceutically acceptable salt thereof is driving its therapeutic benefit in patients suffering from sleep disturbance, in particular one or more of the aspects defined above, either in a causal relationship or at least as a surrogate behavioral marker for the underlying unknown therapeutic mechanism.

Consequently, achieving peak experiences more rapidly, in a larger proportion of patients and with better reproducibility in an individual patient, compared with previously tested psychedelic agents, dosing regimens and administration routes, will lead to a better therapeutic profile.

Further, the present invention also relies on the short duration of action of 5-MeO-DMT and the absence of relevant tolerance (i.e., the absence of diminished or no psychedelic effects after re-administration), as a basis for enabling a dosing regimen with frequent re-administrations (such as more than once daily, or daily), which are designed to increase the rate of occurrence of peak experiences, thereby increasing the therapeutic benefit. Such repeat administrations within short time also allow an intraindividual dose-optimization which reduces the risk of overdosing, which may otherwise lead to somatic side effects, such as the serotonin syndrome, negative psychic reactions, such as flashbacks of the experience at later timepoints, induction of mania or hypomania or to less meaningful psychedelic experiences with few or no memories of the altered state (so-called “white-outs”). Further, starting with a low dose allows familiarization of the patient with the psychedelic experience in general, and allows preparation for the more intense symptoms to occur at the higher doses, which will positively influence the experience at those higher doses. Also, the prospect of being able to initiate treatment with a low dose will increase patient acceptance of the therapeutic approach and improve overall compliance rates on the patient population level.

Frequent re-administrations of a serotonergic psychedelic with the aim to increase the rate and tailor the reproducibility of peak experiences and to improve the therapeutic effect, reduce the side effects and improve the compliance rates may not be possible with other psychedelics, due to the late onset and long duration of psychedelic effects and due to the rapid development of tolerance (i.e. diminished or no psychedelic effects after re-administration) which can last for several days.

A patient as defined herein who suffers from sleep disturbance is treated by administration of 5-MeO-DMT or a pharmaceutically acceptable salt thereof.

In a preferred embodiment, the 5-MeO-DMT is administered as a monotherapy, i.e., the patient does not receive any other treatment for sleep disturbance.

The dosage amount of 5-MeO-DMT administered to a patient, as defined herein, suffering from sleep disturbance, is in the range of about 1 mg to about 10 mg, or any amount of range therein and is administered in the form of a formulation for administration based on a pharmaceutically acceptable salt of 5-MeO-DMT, such as the hydrobromide salt, which weight amount can be calculated from the stated weight amounts of the 5-MeO-DMT free base, assuming that equimolar amounts are used. Useful specific amounts of 5-MeO-DMT are e.g. about 1 mg, about 2 mg, about 3 mg, about 4 mg, about 5 mg, about 6 mg, about 7 mg, about 8 mg, about 9 mg, and about 10 mg. Note that in this specification, when ranges are set forth, such as “about 1 mg to about 10 mg,” the inventor contemplates all discrete values within that range, some of which are specifically mentioned, but all of which are not—simply for the purpose of brevity.

In preferred embodiments the improved methods for the treatment of a patient, as defined herein, suffering from sleep disturbance, with a therapeutically effective amount of 5-MeO-DMT, comprise the occurrence of a clinical response not later than about 2 hours after administration of 5-MeO-DMT.

In preferred embodiments the improved methods for the treatment of a patient, as defined herein, suffering from sleep disturbance, with a therapeutically effective amount of 5-MeO-DMT, comprise the persistence of a clinical response, including a clinical response which occurred not later than about 2 hours after administration of 5-MeO-DMT, until at least about 6 days after the last administration of 5-MeO-DMT, preferably until at least about 14 days after the last administration of 5-MeO-DMT, more preferably until at least about 28 days after the last administration of 5-MeO-DMT.

In preferred embodiments the improved methods for the treatment of a patient, as defined herein, suffering from sleep disturbance, with a therapeutically effective amount of 5-MeO-DMT comprise the administration of more than a single dose of 5-MeO-DMT.

In a preferred embodiment this more than a single dose of 5-MeO-DMT is administered to a patient in one or more treatment blocks, each block consisting of 2 to 7 administrations, with not less than about 1 hour and not more than about 24 hours between each administration within each treatment block, and not less than about 6 days between the end of one treatment block and the start of the next treatment block.

In an even more preferred embodiment this more than a single dose of 5-MeO-DMT is administered to a patient in one or more treatment blocks, each block consisting of 1 to 3 administrations, with about 24 hours between each administration within each treatment block, and not less than about 6 days between the end of one treatment block and the start of the next treatment block.

In a most preferred embodiment this more than a single dose of 5-MeO-DMT is administered to a patient in one or more treatment blocks, each block consisting of 1 to 3 administrations, with about 1 to 4 hours, preferably 1 to 2 hours, between each administration within each treatment block, and not less than about 6 days between the end of one treatment block and the start of the next treatment block.

In an embodiment the dosage amount of the 5-MeO-DMT administered to an individual patient in each of the administrations and in each of the treatment blocks is constant for that individual patient and is selected from about 1 mg to about 10 mg.

In a preferred embodiment the dosage amount of the 5-MeO-DMT administered to an individual patient is selected from about 1 mg to about 2 mg for the first administration within each treatment block, and then increases with each subsequent administration within each treatment block until the earlier of 10 mg being reached or all administrations within that treatment block being administered.

In an even more preferred embodiment the dosage amount of the 5-MeO-DMT administered to an individual patient is selected from about 1 mg to about 2 mg for the first administration within each treatment block, and then increases with each subsequent administration within each treatment block until the earlier of 10 mg being reached or all administrations within that treatment block being administered or the patient having experienced a peak psychedelic experience or the supervising physician having decided that further dose increases are inappropriate based on observed side effects.

For embodiments where the dosage amount increases for subsequent administrations, the dosage amount for the next administration is determined by adding about 0.25 mg to about 3 mg, preferably about 0.5 mg to about 3 mg to the dosage amount of the prior administration. For example, if the dosage amount of the first administration was 1 mg and the dosage amount increase is 3 mg, unless one of the previously mentioned stopping criteria has been reached, then the dosage amount of the second administration will be 4 mg. Preferably, the dosage amount for the third administration will be 7 mg.

In a preferred embodiment the dosage amount of the 5-MeO-DMT administered to an individual patient in each treatment block is selected from about 1 mg to about 3 mg for the first administration, and then increased, unless the patient has already experienced a peak psychedelic experience within that treatment block or the supervising physician has decided that further dose increases are inappropriate based on observed side effects, to a dosage selected from about 4 mg to about 6 mg for the second administration, and from about 7 mg to about 9 mg for the third administration. Useful specific amounts for the first, second and third administration are e.g. about 2 mg, about 5 mg, and about 8 mg.

In an additional preferred embodiment, the dosage amount of the 5-MeO-DMT administered to an individual patient in each treatment block is selected from about 0.5 mg to about 1.5 mg for the first administration, and then increased, unless the patient has already experienced a peak psychedelic experience within that treatment block or the supervising physician has decided that further dose increases are inappropriate based on observed side effects, to a dosage selected from about 1.5 mg to about 2.5 mg for the second administration, and from about 2.5 mg to about 3.5 mg for the third administration. Useful specific amounts for the first, second and third administration are e.g. about 1 mg, about 2 mg, and about 3 mg.

In a further preferred embodiment the dosage amount of the 5-MeO-DMT administered to an individual patient is selected from about 1 mg to about 2 mg for the first administration of the first treatment block, and then increases with each subsequent administration within that first treatment block until the earlier of 10 mg being reached or all administrations within that treatment block being administered or the patient having experienced a peak psychedelic experience or the supervising physician having decided that further dose increases are inappropriate based on observed side effects, with that highest dosage in that first treatment block being used as the dosage for all subsequent treatment blocks and administrations within those subsequent treatment blocks. For example, if the highest dosage in the first treatment block was 8 mg because the patient experienced a peak psychedelic experience at that dose, then the dosage for all subsequent treatment blocks and administrations within those subsequent treatment blocks will be 8 mg.

In a particularly preferred embodiment the dosage amount of the 5-MeO-DMT administered to an individual patient is selected from about 1 mg to about 3 mg for the first administration of the first treatment block, and then increased, unless the patient has already experienced a peak psychedelic experience within that treatment block or the supervising physician has decided that further dose increases are inappropriate based on observed side effects, to a dosage selected from about 4 mg to about 6 mg for the second administration of the first treatment block, and from about 7 mg to about 9 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 2 mg, about 5 mg, and about 8 mg.

In a particularly preferred embodiment the dosage amount of the 5-MeO-DMT administered to an individual patient is selected from about 0.5 mg to about 1.5 mg for the first administration of the first treatment block, and then increased, unless the patient has already experienced a peak psychedelic experience within that treatment block or the supervising physician has decided that further dose increases are inappropriate based on observed side effects, to a dosage selected from about 1.5 mg to about 2.5 mg for the second administration of the first treatment block, and from about 2.5 mg to about 3.5 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 1 mg, about 2 mg, and about 3 mg.

It is understood that a pharmaceutically acceptable salt of 5-MeO-DMT is preferably used in all of the above dosing regimen, and that the appropriate weight amounts of a salt to be administered can be calculated from the stated weight amounts of the free base, assuming that equimolar amounts are used.

According to the invention, 5-MeO-DMT is preferably not administered together with a MAO inhibitor.

The occurrence of a “peak psychedelic experience” in a patient can be identified through achievement of at least 60% of the maximum possible score in each of the four subscales (mystical, positive mood, transcendence of time and space, and ineffability) of the 30-item revised Mystical Experience Questionnaire (MEQ-30) (as described in Barrett F S, J Psychopharmacol. 2015; 29(11):1182-90).

The occurrence of a “peak psychedelic experience” in a patient can also be identified through achievement of at least 60% of the maximum possible score of the Oceanic Boundlessness (OBN) dimension of the Altered States of Consciousness (ASC) questionnaire (as described in Roseman L et al., Front Pharmacol. 2018; 8.974).

In accordance with the invention, the occurrence of a “peak psychedelic experience” in a patient is preferably identified through achievement of a score of at least 75 in the Peak Experience Scale (PES) Total Score, also referred to as the Peak Psychedelic Experience Questionnaire (PPEQ), which averages answers scored by the patient from 0 to 100 for the following three questions: 1. How intense was the experience; 2. To what extent did you lose control; 3. How profound (i.e. deep and significant) was the experience?

### Treatment of Sleep Disturbance and Mental and Nervous System Disorders

According to the invention, idiopathic sleep disturbance as well as sleep disturbance in patients suffering from mental disorders or nervous system disorders can be treated. In patients suffering from sleep disturbance in association with a mental disorder or a nervous system disorder a treatment of sleep disturbance according to the invention leads to an improvement of the condition with which the sleep disturbance is associated.

In many instances, resting state networks involved in sleep disturbance are also involved in the conditions listed above.

5-MeO-DMT has the ability to disrupt established functional connectivity patterns of resting state networks. This disruption leads to a reset of the pathological ill-connected brain connections as the networks reconnect. New, healthy functional connections are established with persistent effects.

The persistence of the effects can be explained by the neuroplasticity-promoting characteristics of 5-MeO-DMT. 5-MeO-DMT in particular fosters the structural and functional plasticity of synapses, i.e., of sites where neurons connect and communicate with each other. 5-MeO-DMT modulates morphogenesis and maturation of dendritic spines so that the formation of new synaptic connections is initiated. These new connections will be strengthened or weakened or even be eliminated, dependent on activity.

New synapses ultimately formed will in turn influence the activity patterns of the neurons. The inventors conclude that such reciprocal structural and functional modifications contribute to a proper establishment of networks and the persistence of effects after administration of 5-MeO-DMT.

From a biochemical perspective, 5-MeO-DMT interacts, among others, with 5-HT receptors. 5-HT receptors, receptors for the neurotransmitter serotonin or 5-hydroxytryptamine (5-HT), are found throughout the central and the peripheral nervous system. A wide range of physiological and pathological functions are mediated via these receptors.

In the brain, seven types of 5-HT receptors which can be further separated into several subtypes are expressed. The various types and subtypes show distinct spatial distributions.

5-MeO-DMT interacts with several of the 5-HT receptors. These receptors are involved in mediating effects of 5-MeO-DMT on resting state networks and neuronal plasticity. Besides 5-HTA1 and 5-HT2A discussed above, 5-MeO-DMT also interacts with the 5-HT7 receptor. 5-MeO-DMT act as an agonist on this receptor and shows a high (nanomolar) binding affinity.

The 5-HT7 receptor has a role in neurogenesis, synaptogenesis and dendritic spine formation. It is, among other things, associated with central processes such as learning and memory, with sleep regulation and circadian rhythm and with nociception.

The 5-HT7 receptor is in particular expressed in the spinal cord, raphe nuclei, thalamus, hypothalamus including the suprachiasmatic nucleus, hippocampus, prefrontal cortex, striatal complex, amygdala and in the Purkinje neurons of the cerebellum.

The suprachiasmatic nucleus is the central pacemaker of the circadian timing system. It coordinates circadian rhythms in various brain regions. Disruption of this coordination will result in disease states, in particular disease states involving sleep disturbance. In patients suffering from sleep disturbance resting state functional connectivity analysis reveals alterations in functional connectivity between the suprachiasmatic nucleus and regions within the default mode network.

The expression of the 5-HT7 receptor in the suprachiasmatic nucleus corresponds to the function of the receptor in regulation of sleep/wake cycles. The inventors consider that this allows treatment of patients suffering from sleep disturbance by 5-MeO-DMT which acts on the receptor.

The inventors consider that binding of 5-MeO-DMT to the 5-HT7 receptor as one mediator of the pharmacological effects of 5-MeO-DMT, which involve functional connectivity “resets” of networks and neuroplasticity effects, contributes to the beneficial effects of 5-MeO-DMT in the treatment of patients suffering from sleep disturbance.

The inventors further consider that binding of 5-MeO-DMT to the 5-HT7 receptor as well as to the 5-HT1A receptor as two mediators of effects exerted by 5-MeO-DMT, which include functional connectivity “resets” of networks and neuroplasticity effects, allows achieving beneficial effects also in patients suffering from other symptoms or conditions, such as cognitive dysfunction, anxiety, psychomotor retardation, negative thinking or social/emotional withdrawal. This is supported by the clinical results demonstrated in studies referred to herein.

A treatment according to the invention will lead to an improvement of the sleep disturbance and, if the patient treated suffers from a mental disorder or a nervous system disorder, also of that disorder.

Clinical data from studies of patients suffering from Treatment Resistant Depression (TRD) or Postpartum Depression (PPD) confirm that sleep disturbance can successfully be treated by the administration of 5-MeO-DMT.

In the TRD studies, which are described in more detail in the example section below, among others the MADRS item “reduced sleep”, which reflects insomnia, was assessed.

The MADRS item “reduced sleep” represents the experience of reduced duration or depth of sleep compared to the subject's own normal pattern when well. A score of 0 is assigned when the subject sleeps as usual. A score of 2 reflects slight difficulty dropping off to sleep or slightly reduced, light or fitful sleep. A score of 4 means that sleep is reduced or broken by at least two hours. A score of 6 means less than two or three hours sleep.

In the study group receiving the individualized dosing regimen, the aggregated score for the MADRS item “reduced sleep” across all 8 patients was 25 at base line. At day 1 after treatment, the earliest timepoint for assessing an impact of the treatment on sleep, it was reduced to 12 which corresponds to an improvement of 13 points or 52%. At day 7 after treatment, it was reduced to 9 which corresponds to an improvement of 16 points or 64%.

In the 12 mg group, the aggregated score for the MADRS item “reduced sleep” across all 4 patients was 12 at base line. At day 1 after treatment, it was reduced to 10 which corresponds to an improvement of 2 points or 17%. At day 7 after treatment, it was reduced to 6 which corresponds to an improvement of 6 points or 50%.

Thus, the score of the scale item that is of particular relevance to sleep disturbance, “reduced sleep”, is markedly improved. The inventors conclude that 5-MeO-DMT can be used to treat sleep disturbance, in particular patients suffering from a mental disorder or a nervous system disorder.

### Idiopathic Sleep Disturbance

Treating a patient suffering from idiopathic sleep disturbance with 5-MeO-DMT or a pharmaceutically acceptable salt thereof reduces or eliminates the idiopathic sleep disturbance.

The reduction or elimination of idiopathic sleep disturbance is observed on day 1, for instance after about 24 hours; on day 7; on day 14; and/or on day 28 after the last administration of 5-MeO-DMT or a pharmaceutically acceptable salt thereof.

The reduction or elimination of idiopathic sleep disturbance preferably occurs not later than about 24 hours after the last administration of 5-MeO-DMT or a pharmaceutically acceptable salt thereof. The reduction or elimination of idiopathic sleep disturbance preferably persists until at least 6 days; in particular until at least 14 days; more preferably until at least 28 days after the last administration of 5-MeO-DMT or a pharmaceutically acceptable salt thereof.

In an embodiment, the reduction or elimination of idiopathic sleep disturbance is observed on day 7 after the last administration of 5-MeO-DMT or a pharmaceutically acceptable salt thereof and preferably persists until at least 14 days, more preferably until at least 28 days.

In cases of idiopathic sleep disturbance, a clinical response may be reflected by a reduction in the Clinical Global Impression-Severity (CGI-S) score. According to the invention, a reduction in the CGI-S score means that the CGI-S is reduced by at least 1. Preferably, the CGI-S is reduced by at least 2 and/or to a score of 0. It is especially preferred if the CGI-S is reduced by at least 3 and/or to a score of 0.

An improvement of idiopathic sleep disturbance as reflected by a reduction in the CGI-S score is observed on day 1, for instance after about 24 hours; on day 7; on day 14; and/or on day 28 after the last administration of 5-MeO-DMT or a pharmaceutically acceptable salt thereof.

An improvement of idiopathic sleep disturbance as reflected by a reduction in the CGI-S score, occurs not later than about 24 hours after the last administration of 5-MeO-DMT or a pharmaceutically acceptable salt thereof.

An improvement in idiopathic sleep disturbance as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in idiopathic sleep disturbance as reflected by a reduction in the CGI-S score is observed on day 7 after the last administration of 5-MeO-DMT or a pharmaceutically acceptable salt thereof and preferably persists until at least 14 days, more preferably until at least 28 days.

In cases of idiopathic sleep disturbance, an improvement in sleep disturbance, as reflected by at least a score of “much improved” in the Clinical Global Impression-Improvement (CGI-1) score or the Patient Global Impression-Improvement (PGI-I) score, is observed on day 1, for instance after about 24 hours; on day 7; on day 14; and/or on day 28 after the last administration of 5-MeO-DMT or a pharmaceutically acceptable salt thereof.

An improvement in idiopathic sleep disturbance, as reflected by at least a score of “much improved” in the CGI-I score or the PGI-I score, preferably occurs not later than about 24 hours after the last administration of 5-MeO-DMT or a pharmaceutically acceptable salt thereof.

The improvement in sleep disturbance, as reflected by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

In an embodiment, an improvement in idiopathic sleep disturbance as reflected by at least a score of “much improved” in the CGI-I score or the PGI-I score is observed on day 7 after the last administration of 5-MeO-DMT or a pharmaceutically acceptable salt thereof and preferably persists until at least 14 days, more preferably until at least 28 days.

Improvements in cases of idiopathic sleep disturbance may also be assessed by any other scale reflecting changes in sleep quality or quantity, as indicated above, for instance the Pittsburgh Sleep Quality Index (PSQI).

If treatment outcome is assessed using the PSQI, treatment success is indicated (i) by a decrease of the global score, preferably (ii) by a decrease to 5 or below. The recall period applied does not start earlier than the time point when acute psychedelic experiences have subsided after the last administration.

An improvement in idiopathic sleep disturbance, as reflected by a decrease of the PSQI global score, in particular by a decrease to 5 or below, preferably occurs not later than about 24 hours after the last administration of 5-MeO-DMT or a pharmaceutically acceptable salt thereof.

Such an improvement in idiopathic sleep disturbance as reflected by a decrease of the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

An improvement of idiopathic sleep disturbance as reflected by a reduction in the PSQI global score, in particular by a decrease to 5 or below, is observed on day 1, for instance after about 24 hours; on day 7; on day 14; and/or on day 28 after the last administration of 5-MeO-DMT or a pharmaceutically acceptable salt thereof.

In an embodiment, an improvement of idiopathic sleep disturbance as reflected by a reduction in the PSQI global score, in particular by a decrease to 5 or below, is observed on day 7 and preferably persists until day 14, more preferably until day 28.

Sleep Disturbance Associated with Mental and Nervous System Disorders

While sleep disturbance may be considered a condition deserving treatment independent of any other condition, disorder or symptom an individual may suffer from, several mental disorders and nervous system disorders are associated with sleep disturbance. Notably, the relationship between sleep and a mental or nervous system disorder is often bidirectional. Not only can mental or nervous system disorders have a negative impact on a healthy sleep pattern but sleep disturbance can also be a contributing factor to the onset, progression, and prognosis of mental health or nervous system disorders.

The treatment according to the invention reduces or eliminates sleep disturbance and preferably also improves the associated mental disorder or nervous system disorder.

### Disorders Characterized by Depressive Episodes

There are several disorders which are characterized by depressive episodes.

A depressive episode is a period of depressed mood and/or loss of pleasure in most activities.

For instance, according to DSM-V, a Major Depressive Episode is characterized by five or more symptoms that have been present during the same 2-week period and represent a change from previous functioning; at least one of the symptoms being either (1) depressed mood or (2) loss of interest or pleasure.

The patient suffering from a disorder characterized by depressive episodes may suffer from a treatment resistant form of the disorder.

During a depressive episode, a patient often suffers from insomnia or hypersomnia.

Sleep disturbance is included as a diagnostic criterion for disorders characterized by depressive episodes. It may be assessed as part of the determination of the MADRS or the HAM-D.

The Montgomery-Åsberg Depression Rating Scale (MADRS) is a ten-item diagnostic questionnaire used to measure the severity of depressive episodes in patients with mood disorders. Higher MADRS scores indicate more severe depression.

Items are apparent sadness; reported sadness; inner tension; reduced sleep; reduced appetite; concentration difficulties; lassitude; inability to feel; pessimistic thoughts; and suicidal thoughts and each item yields a score of 0 to 6. The overall score ranges from 0 to 60.

The item reduced sleep reflects sleep disturbance, in particular the experience of reduced duration or depth of sleep compared to the subject's own normal pattern when well. A score of 0 is assigned if the patient sleeps as usual. A score of 2 is assigned if the patient suffers from slight difficulty dropping off to sleep or slightly reduced, light or fitful sleep. The score is 4 if sleep is reduced or broken by at least two hours. The score is 6 in case of less than two or three hours sleep.

The Hamilton Rating Scale for Depression (HAM-D) allows clinicians to assess the nature and severity of mood disorders in patient populations. The scale is comprised of 21 items for inquiry, though only the first 17 (depressed mood; feelings of guilt; suicide; initial insomnia; insomnia during the night; delayed insomnia; work and interests; retardation; agitation; psychiatric anxiety; somatic anxiety; gastrointestinal somatic symptoms; general somatic symptoms; genital symptoms; hypochondriasis; weight loss; insight) are used in scoring.

For the majority of questions, scores range from 0 to 4, with 4 representing more acute signs of depression. Several questions have ranges that extend only as high as 2. A total score is tallied and can then be compared with previous scores or can be contrasted with a pre-defined cut-off score.

Initial insomnia reflects insomnia early in the night. It is scored 0 if the patient has no difficulty falling asleep; 1 if the patient complains of occasional difficulty falling asleep, i.e., more than ½ hour; 2 if the patient complains of nightly difficulty falling asleep.

Insomnia, during the night, i.e., in the middle of the night, is scored 0 if there is no difficulty; 1 if the patient complains of being restless and disturbed during the night; 2 if the patient wakes during the night (any getting out of bed, except for purposes of voiding, rates 2).

Delayed insomnia reflects insomnia in the early hours of the morning. The score is 0 if there is no difficulty; 1 if the patient wakes in early hours of the morning but goes back to sleep; 2 if the patient is unable to fall asleep again if he/she gets out of bed.

In patients suffering from a disorder characterized by depressive episodes altered functional connectivity is observed within and/or between several brain regions implicated in processing, regulation, emotional memory; cognitive processes related to rumination; impaired concentration and physiological arousal.

Treating a patient suffering from a disorder characterized by depressive episodes, including a treatment resistant form of the disorder, and associated sleep disturbance with 5-MeO-DMT or a pharmaceutically acceptable salt thereof reduces or eliminates the sleep disturbance and leads to an improvement of the disorder characterized by depressive episodes.

The reduction or elimination of sleep disturbance in a patient suffering from a disorder characterized by depressive episodes is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from a disorder characterized by depressive episodes occurs not later than about 24 hours after the last administration of 5-MeO-DMT or a pharmaceutically acceptable salt thereof. The reduction or elimination of sleep disturbance preferably persists until at least 6 days; in particular until at least 14 days; more preferably until at least 28 days after the last administration of 5-MeO-DMT or a pharmaceutically acceptable salt thereof.

In an embodiment, the reduction or elimination of sleep disturbance in a patient suffering from a disorder characterized by depressive episodes is observed on day 7 after the last administration of 5-MeO-DMT or a pharmaceutically acceptable salt thereof and preferably persists until at least 14 days, more preferably until at least 28 days.

An improvement in sleep disturbance in a patient suffering from a disorder characterized by depressive episodes, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from a disorder characterized by depressive episodes, as reflected by a reduction in the CGI-S score, occurs not later than about 24 hours after the last administration of 5-MeO-DMT or a pharmaceutically acceptable salt thereof. An improvement in sleep disturbance in a patient suffering from a disorder characterized by depressive episodes, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from a disorder characterized by depressive episodes, as reflected by a reduction in the CGI-S score, is observed on day 7 after the last administration of 5-MeO-DMT or a pharmaceutically acceptable salt thereof and preferably persists until at least 14 days, more preferably until at least 28 days.

The reduction or elimination of sleep disturbance in a patient suffering from a disorder characterized by depressive episodes, in particular of reduced sleep, is reflected by at least an improvement in the score of the MADRS item reduced sleep on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from a disorder characterized by depressive episodes, in particular of reduced sleep, as reflected by an improvement in the score of the MADRS item reduced sleep occurs not later than about 24 hours after the last administration of 5-MeO-DMT or a pharmaceutically acceptable salt thereof. The reduction or elimination of sleep disturbance in a patient suffering from a disorder characterized by depressive episodes, in particular of reduced sleep, as reflected by an improvement in the score of the MADRS item reduced sleep preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from a disorder characterized by depressive episodes, in particular of reduced sleep, as reflected by an improvement in the score of the MADRS item reduced sleep, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from a disorder characterized by depressive episodes, in particular of reduced sleep, is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from a disorder characterized by depressive episodes, in particular of reduced sleep, as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from a disorder characterized by depressive episodes, in particular of reduced sleep, as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from a disorder characterized by depressive episodes, in particular of reduced sleep, as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is closely linked to disorders characterized by depressive episodes. An improvement in sleep disturbance will therefore also lead to an improvement of a disorder characterized by depressive episodes. Since sleep disturbance furthermore also affects other aspects of a disorder characterized by depressive episodes, the inventors conclude that the improvement in sleep disturbance, in particular the reduction or elimination of reduced sleep, will additionally contribute to an overall improvement of the disorder characterized by depressive episodes.

An improvement of the disorder characterized by depressive episodes in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the disorder characterized by depressive episodes in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the disorder characterized by depressive episodes in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of the disorder characterized by depressive episodes in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Major Depressive Disorder (MDD) is a mood disorder that causes a persistent feeling of sadness and loss of interest. It affects how a person feels, thinks, and behaves and can lead to a variety of emotional and physical problems.

The patient may suffer from moderate or severe MDD as indicated by a Montgomery-Åsberg Depression Rating Scale (MADRS) score of 20 or more or by a Hamilton Depression Rating Scale (HAM-D) score of 17 or more. It is further considered that the patient may suffers from severe major depressive disorder as indicated by a Montgomery-Åsberg Depression Rating Scale (MADRS) score of 35 or more or by a Hamilton Depression Rating Scale (HAM-D) score of 25 or more.

The patient suffering from MDD may suffer from a treatment resistant form of the disorder (TRD).

In patients suffering from MDD dysfunctional connectivity and regulation within and/or between multiple resting-state networks including the DMN, salience network, executive control network and limbic network is observed. Functional connectivity differs significantly from that observed in healthy controls.

As indicated above, dysfunctional connectivity of resting state networks is also associated with sleep disturbance, such as insomnia and hypersomnia.

An estimated ¾ of all patients with MDD suffer from a form of sleep disturbance such as insomnia or hypersomnia, sometimes both in the same episode. In the DSM-5 criteria, sleep disturbance is one of 9 symptoms of which at least 5 need to be present for a certain period of time to satisfy a diagnosis of MDD.

Sleep disturbance in the context of MDD is associated with a greater risk of suicide and a significant reduction in the quality of the life of the patient.

Treating a patient suffering from MDD, including a treatment resistant form of the disorder, and associated sleep disturbance with 5-MeO-DMT or a pharmaceutically acceptable salt thereof reduces or eliminates the sleep disturbance and leads to an improvement of MDD.

The reduction or elimination of sleep disturbance in a patient suffering from MDD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from MDD occurs not later than about 24 hours after the last administration of 5-MeO-DMT or a pharmaceutically acceptable salt thereof. The reduction or elimination of sleep disturbance preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from MDD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from MDD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from MDD, as reflected by a reduction in the CGI-S score, occurs not later than about 24 hours after the last administration of 5-MeO-DMT or a pharmaceutically acceptable salt thereof. An improvement in sleep disturbance in a patient suffering from MDD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from MDD, as reflected by a reduction in the CGI-S score, is observed on day 7 after the last administration of 5-MeO-DMT or a pharmaceutically acceptable salt thereof and preferably persists until at least 14 days, more preferably until at least 28 days.

The reduction or elimination of sleep disturbance in a patient suffering from MDD, in particular of reduced sleep, is reflected by at least an improvement in the score of the MADRS item reduced sleep on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from MDD, in particular of reduced sleep, as reflected by at least an improvement in the score of the MADRS item reduced sleep occurs not later than about 24 hours after the last administration of 5-MeO-DMT or a pharmaceutically acceptable salt thereof. The reduction or elimination of sleep disturbance in a patient suffering from MDD, in particular of reduced sleep, as reflected by at least an improvement in the score of the MADRS item reduced sleep preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from MDD, in particular of reduced sleep, as reflected by an improvement in the score of the MADRS item reduced sleep, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from MDD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from MDD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from MDD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from MDD, in particular of reduced sleep, as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is closely linked to MDD. An improvement in sleep disturbance will therefore also lead to an improvement of MDD. Since sleep disturbance furthermore also affects other aspects of MDD, the inventors conclude that the improvement in sleep disturbance, in particular the reduction or elimination of reduced sleep, will additionally contribute to an overall improvement of MDD.

An improvement of the Major Depressive Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Major Depressive Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Major Depressive Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of MDD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Sleep Disturbance has Also been Associated with Postpartum Depression (PPD).

Over 50% of women may experience short-lasting low mood or tearfulness after childbirth. However, a subset of women may develop PPD—a debilitating mood disorder occurring during pregnancy or within 4 weeks following delivery. Epidemiological studies estimate that the prevalence rate of PPD is about 15%.

A patient may suffer from moderate or severe PPD as indicated by a Montgomery-Åsberg Depression Rating Scale (MADRS) score of 20 or more or by a Hamilton Depression Rating Scale (HAM-D) score of 16 or more. It is further considered that the patient may suffer from severe PPD as indicated by a Montgomery-Åsberg Depression Rating Scale (MADRS) score of 35 or more or by a Hamilton Depression Rating Scale (HAM-D) score of 27 or more.

A patient treated according to the invention may have a Montgomery-Åsberg Depression Rating Scale (MADRS) score of 20 or more or a 17-item Hamilton Depression Rating Scale (HAM-D) score of 16 or more.

Further, a patient treated according to the invention may have a MADRS score of 28 or more or a HAM-D score of 22 or more.

Still further, a patient treated according to the invention may have a MADRS score of 35 or more or a HAM-D score of 25 or more.

The patient suffering from PPD may suffer from a treatment resistant form of the disorder.

Depression during the postpartum period not only affects the mother's overall wellbeing, but it also affects how a mother interacts with her child and thus how the child develops. Sleep disturbance is a commonly reported symptom during pregnancy and is highly linked to the presence of depressive symptoms. Women with poorer sleep quality are 3.34 times more likely to suffer from depression than those with good sleep quality.

The presence of sleep disturbance during the pregnancy period is itself a predictive symptom of PPD.

Studies note that change in sleep quality rather than hormonal milieu are associated with PPD recurrence and severity.

PPD can be assessed by the Edinburgh Postnatal Depression Scale (EPDS), a scale evaluating how the mother has felt the past 7 days. Insomnia as a consequence of unhappiness is one of out of 10 questions that is assessed by the questionnaire. Rating ranges from “0” “No, not at all” to “3” “Yes, most of the time”. With a threshold level of a total score of 13, sleep disturbances can be considered as a fundamental part of PPD diagnosis.

In patients with PPD, significant changes in neural activity in brain regions important for self-regulation, empathy, emotion, and cognition are observed. PPD is associated with dysfunctional connectivity of resting state networks, for instance, within and/or between the default mode network and frontoparietal network.

Treating a patient suffering from PPD, including a treatment resistant form of the disorder, and associated sleep disturbance with 5-MeO-DMT or a pharmaceutically acceptable salt thereof reduces or eliminates the sleep disturbance and leads to an improvement of PPD.

The reduction or elimination of sleep disturbance in a patient suffering from PPD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from PPD occurs not later than about 24 hours after the last administration of 5-MeO-DMT or a pharmaceutically acceptable salt thereof. The reduction or elimination of sleep disturbance preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from PPD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from PPD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from PPD, as reflected by a reduction in the CGI-S score, occurs not later than about 24 hours after the last administration of 5-MeO-DMT or a pharmaceutically acceptable salt thereof. An improvement in sleep disturbance in a patient suffering from PPD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from PPD, as reflected by a reduction in the CGI-S score, is observed on day 7 after the last administration of 5-MeO-DMT or a pharmaceutically acceptable salt thereof and preferably persists until at least 14 days, more preferably until at least 28 days.

The reduction or elimination of sleep disturbance in a patient suffering from PPD, in particular of reduced sleep, is reflected by at least an improvement in the score of the MADRS item reduced sleep on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from PPD, in particular of reduced sleep, as reflected by an improvement in the score of the MADRS item reduced sleep occurs not later than about 24 hours after the last administration of 5-MeO-DMT or a pharmaceutically acceptable salt thereof. The reduction or elimination of sleep disturbance in a patient suffering from PPD, in particular of reduced sleep, as reflected by an improvement in the score of the MADRS item reduced sleep preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from PPD, in particular of reduced sleep, as reflected by an improvement in the score of the MADRS item reduced sleep, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from PPD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from PPD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from PPD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from PPD, in particular of reduced sleep, as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is closely linked to PPD. An improvement in sleep disturbance will therefore also lead to an improvement of PPD. Since sleep disturbance furthermore also affects other aspects of PPD, the inventors conclude that the improvement in sleep disturbance, in particular the reduction or elimination of reduced sleep, will additionally contribute to an overall improvement of PPD.

An improvement of the Postpartum Depression in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Postpartum Depression in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Postpartum Depression in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of PPD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

In an embodiment, an improvement of PPD in a patient also suffering from associated sleep disturbance, as reflected by an improvement on the Edinburgh Postnatal Depression Scale (EPDS), is observed on day 7 after the last administration of 5-MeO-DMT or a pharmaceutically acceptable salt thereof and preferably persists until at least 14 days, more preferably until at least 28 days.

Sleep disturbance moreover compromises maternal functioning.

Maternal functioning can, for instance, be assessed using the Barkin Index of Maternal Functioning (BIMF). This index was designed to measure functioning in the year after childbirth. The BIMF is a 20-item self-report measure of functioning. Each item is assigned a score between 0 and 6 so that the maximum total score is 120. The higher the score, the better maternal functioning is rated.

The BIMF identifies the key functional domains of a mother during the postnatal period as: self-care, infant care, mother-child interaction, psychological wellbeing of the mother, social support, management, and adjustment.

A BIMF score of 95 or below is considered herein as representing slightly compromised maternal functioning, a score of 80 or below is considered herein as representing compromised maternal functioning, a score of 65 or below is considered herein as representing severely compromised maternal functioning.

The inventors have determined that increases in the score of the MADRS item “reduced sleep” have negative impacts on both aspects of maternal functioning (maternal competence relating to interactions with the infant(s) as well as maternal self-care). Increased scores in the MADRS item “reduced sleep” impair self-care, psychological wellbeing and management.

Increased scores in the MADRS item reduced sleep impair psychological wellbeing, social support and management. Conversely, improvements regarding this MADRS item lead to improvements in maternal functioning, in particular in the BIMF functional domains self-care, psychological well-being and/or management.

The inventors conclude that 5-MeO-DMT can be used to treat PPD patients to achieve an improvement of sleep disturbance, in particular a reduction or elimination of reduced sleep.

The inventors furthermore conclude that a reduction or elimination of reduced by treating a PPD patient does not only lead to a reduction in the MADRS total score, but also to an improvement in maternal functioning, as reflected by an increase in the BIMF score.

The BIMF total score is improved by 10% or more, preferably by 20% or more.

The improvement of maternal functioning in a patient suffering from PPD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from PPD, as reflected by at least an improvement in the BIMF total score, occurs not later than about 6 days after the last administration of 5-MeO-DMT or a pharmaceutically acceptable salt thereof. The improvement of maternal functioning, as reflected by at least an improvement in the BIMF total score, preferably persists until at least 14 days; more preferably until at least 28 days after the last administration of 5-MeO-DMT or a pharmaceutically acceptable salt thereof.

### Persistent Depressive Disorder

Persistent Depressive Disorder, also referred to as dysthymia, is a chronic form of depression. It is diagnosed if depression is present for most of the day for the majority of days over at least a two-year period. Any symptom-free period is less than 2 months.

While depressed, two or more of the following must be present: 1. Hopelessness; 2. Energy low or fatigue; 3. Self-esteem is low; 4. Sleep decreased (insomnia) or increased (hypersomnia): 5. Appetite poor, or overeating; 6. Difficulty making decisions or poor concentration.

The patient suffering from Persistent Depressive Disorder may suffer from a treatment resistant form of the disorder.

In patients suffering from Persistent Depressive Disorder altered functional connectivity is observed within and/or between several brain regions implicated in processing, regulation, emotional memory; cognitive processes related to rumination; impaired concentration and physiological arousal. Dysfunctional connectivity is observed within and/or between the DMN, salience network, executive control network and limbic network. Functional connectivity differs significantly from that observed in healthy controls.

Treating a patient suffering from Persistent Depressive Disorder, including a treatment resistant form of the disorder, and associated sleep disturbance with 5-MeO-DMT or a pharmaceutically acceptable salt thereof reduces or eliminates the sleep disturbance and leads to an improvement of Persistent Depressive Disorder.

The reduction or elimination of sleep disturbance in a patient suffering from Persistent Depressive Disorder is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Persistent Depressive Disorder occurs not later than about 24 hours after the last administration of 5-MeO-DMT or a pharmaceutically acceptable salt thereof. The reduction or elimination of sleep disturbance preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Persistent Depressive Disorder is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Persistent Depressive Disorder, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Persistent Depressive Disorder, as reflected by a reduction in the CGI-S score, occurs not later than about 24 hours after the last administration of 5-MeO-DMT or a pharmaceutically acceptable salt thereof. An improvement in sleep disturbance in a patient suffering from Persistent Depressive Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Persistent Depressive Disorder, as reflected by a reduction in the CGI-S score, is observed on day 7 after the last administration of 5-MeO-DMT or a pharmaceutically acceptable salt thereof and preferably persists until at least 14 days, more preferably until at least 28 days.

The reduction or elimination of sleep disturbance in a patient suffering from Persistent Depressive Disorder, in particular of reduced sleep, is reflected by at least an improvement in the score of the MADRS item reduced sleep on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Persistent Depressive Disorder, in particular of reduced sleep, as reflected by at least an improvement in the score of the MADRS item reduced sleep occurs not later than about 24 hours after the last administration of 5-MeO-DMT or a pharmaceutically acceptable salt thereof. The reduction or elimination of sleep disturbance in a patient suffering from Persistent Depressive Disorder, in particular of reduced sleep, as reflected by at least an improvement in the score of the MADRS item reduced sleep preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Persistent Depressive Disorder, in particular of reduced sleep, as reflected by an improvement in the score of the MADRS item reduced sleep, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Persistent Depressive Disorder is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Persistent Depressive Disorder as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Persistent Depressive Disorder as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Persistent Depressive Disorder, in particular of reduced sleep, as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is closely linked to Persistent Depressive Disorder. An improvement in sleep disturbance will therefore also lead to an improvement of Persistent Depressive Disorder. Since sleep disturbance furthermore also affects other aspects of Persistent Depressive Disorder, the inventors conclude that the improvement in sleep disturbance, in particular the reduction or elimination of reduced sleep, will additionally contribute to an overall improvement of Persistent Depressive Disorder.

An improvement of the Persistent Depressive Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Persistent Depressive Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Persistent Depressive Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Persistent Depressive Disorder in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Sleep Disturbance is Also a Common Symptom of Seasonal Affective Disorder.

Seasonal Affective Disorder is a mood disorder with seasonal pattern, with symptoms often beginning in autumn and remitting in spring. Many people experience sadness, hopelessness, a loss of interest in activities, tiredness, and social withdrawal.

The patient suffering from Seasonal Affective Disorder may suffer from a treatment resistant form of the disorder.

Patients with sSAffective Disorder often feel excessively sleepy during the day and sleep longer than usual at night, have difficulty waking from a long sleep or feel the need to nap repeatedly throughout the day, even though, napping may not provide relief from feeling sleepy. Also, nightmares are common among people with Seasonal Affective Disorder.

Sleep length in dependence of month and season is a central item of assessing Seasonal Affective Disorder by the seasonal pattern assessment questionnaire (SPAQ). Subjects are asked to score seasonal changes they have experienced in sleep, socialization, mood, weight, appetite, and energy, whereby sleep is queried in three out of six questions.

Resting state activity involved in sensorimotor, attention, and visual processing is altered in patients with Seasonal Affective Disorder compared to healthy controls.

In patients suffering from Seasonal Affective Disorder altered functional connectivity is observed within and/or between several brain regions implicated in processing, regulation, emotional memory; cognitive processes related to rumination; impaired concentration and physiological arousal. Dysfunctional connectivity is observed within and/or between the DMN, salience network, executive control network and limbic network. Functional connectivity differs significantly from that observed in healthy controls.

Treating a patient suffering from Seasonal Affective Disorder, including a treatment resistant form of the disorder, and associated sleep disturbance with 5-MeO-DMT or a pharmaceutically acceptable salt thereof reduces or eliminates the sleep disturbance and leads to an improvement of Seasonal Affective Disorder.

The reduction or elimination of sleep disturbance in a patient suffering from Seasonal Affective Disorder is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Seasonal Affective Disorder occurs not later than about 24 hours after the last administration of 5-MeO-DMT or a pharmaceutically acceptable salt thereof. The reduction or elimination of sleep disturbance in a patient suffering from Seasonal Affective Disorder preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Seasonal Affective Disorder is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Seasonal Affective Disorder, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Seasonal Affective Disorder, as reflected by a reduction in the CGI-S score, occurs not later than about 24 hours after the last administration of 5-MeO-DMT or a pharmaceutically acceptable salt thereof. An improvement in sleep disturbance in a patient suffering from Seasonal Affective Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Seasonal Affective Disorder, as reflected by a reduction in the CGI-S score, is observed on day 7 after the last administration of 5-MeO-DMT or a pharmaceutically acceptable salt thereof and preferably persists until at least 14 days, more preferably until at least 28 days.

The reduction or elimination of sleep disturbance in a patient suffering from Seasonal Affective Disorder, in particular of reduced sleep, is reflected by at least an improvement in the score of the MADRS item reduced sleep on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Seasonal Affective Disorder, in particular of reduced sleep, as reflected by an improvement in the score of the MADRS item reduced sleep occurs not later than about 24 hours after the last administration of 5-MeO-DMT or a pharmaceutically acceptable salt thereof. The reduction or elimination of sleep disturbance in a patient suffering from Seasonal Affective Disorder, in particular of reduced sleep, as reflected by an improvement in the score of the MADRS item reduced sleep preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Seasonal Affective Disorder, in particular of reduced sleep, as reflected by an improvement in the score of the MADRS item reduced sleep, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Seasonal Affective Disorder is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Seasonal Affective Disorder as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Seasonal Affective Disorder as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Seasonal Affective Disorder, in particular of reduced sleep, as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is closely linked to Seasonal Affective Disorder. An improvement in sleep disturbance will therefore also lead to an improvement of Seasonal Affective Disorder. Since sleep disturbance furthermore also affects other aspects of Seasonal Affective Disorder, the inventors conclude that the improvement in sleep disturbance, in particular the reduction or elimination of reduced sleep, will additionally contribute to an overall improvement of Seasonal Affective Disorder.

An improvement of the Seasonal Affective Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Seasonal Affective Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Seasonal Affective Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Seasonal Affective Disorder in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Bipolar Disorder (BD) is a mental health condition characterized by extreme mood swings that include emotional lows (major depressive episodes) and highs (manic or hypomanic episodes). BD is a recurrent chronic disorder that affects more than 1% of the world's population irrespective of ethnic origin or socioeconomic status.

The patient suffering from BD may suffer from a treatment resistant form of the disorder.

BD is classified as Bipolar I Disorder if there has been at least one manic episode, with or without depressive episodes. It is classified as Bipolar II Disorder if there has been at least one hypomanic episode (but no full manic episodes) and one major depressive episode. If these symptoms are due to drugs or medical problems, they are not diagnosed as Bipolar Disorder.

The patient suffering from BD, whether diagnosed with Bipolar II Disorder or with Bipolar I Disorder, in particular suffers from a current major depressive episode.

The severity of a current major depressive episode may be assessed using the Montgomery-Åsberg Depression Rating Scale (MADRS). The patient may have a total score of equal to or greater than 19, such as greater or equal than 24, in particular greater or equal than 37.

Alternatively or in addition, the patient may have a Bipolar Depression Rating Scale (BDRS) total score of 19, such as greater or equal than 24, in particular greater or equal than 37.

Sleep disturbance including variability in total sleep time (insomnia/hypersomnia) and circadian rhythm abnormalities have in particular been noted in patients suffering from BD, including Bipolar I Disorder and Bipolar II Disorder.

Decreased need for sleep is often an indicator of the onset of a manic episode.

The disturbance of normal sleep-wake cycles has been implicated in the psychopathology of Bipolar Disorder, with dysregulated circadian systems regarded as a biomarker of Bipolar Disorder.

During a manic episode, sleep disturbance is seen in 69-99% of patients.

Sleep disturbances contribute to early episode relapse and a lower quality of life (decreased healthy behaviours seen and increased risk of suicide).

The Bipolar Depression Rating Scale (BDRS) is designed to measure the severity of depressive symptoms in bipolar depression. The BDRS is validated for clinical use by trained raters. Based on a clinical interview, the BDRS items rate the severity of depressive and/or mixed symptoms expressed by patients currently and during the past few days. If there is a discordance between symptoms currently and the last few days, the rating should reflect current symptoms. The scale contains 20 questions and the maximum score possible is 60. Higher scores indicate greater severity.

The questions address depressed mood; sleep disturbance; appetite disturbance; reduced social engagement; reduced energy and activity; reduced motivation; impaired concentration and memory; anxiety; anhedonia; affective flattening; feelings of worthlessness; feelings of helplessness and hopelessness; suicidal ideation; feelings of guilt; psychotic symptoms; irritability; lability; increased motor drive; increased speech; agitation.

Each of these aspects is assessed and assigned a score of 0, 1, 2 or 3.

Sleep disturbance (sleep dysregulation) is assessed based on the change in total amount of sleep over a 24-hour cycle, rated independent of the effect of external factors. It can either take the form of insomnia (reduction in total sleep time) or the form of hypersomnia (increase in total sleep time, inclusive of daytime sleep).

The rating for insomnia (reduction in total sleep time) involves scores of 0 (no reduction in total sleep time); 1 (mild; reduction up to 2 hours); 2 (moderate; 2-4 hours); 3 (severe; more than 4 hours).

The alternative rating for hypersomnia (increase in total sleep time, inclusive of daytime sleep) involves scores of 0 (no increase in total sleep time, inclusive of daytime sleep); 1 (mild; less than 2 hours, or normal amount but non-restorative); 2 (moderate; 2-4 hours); 3 (severe; greater than 4 hours).

Patients suffering from Bipolar Disorder show characteristic aberrant intrinsic organization and interconnectivity of resting state networks. In comparison with healthy controls, resting state functional magnetic resonance imaging studies demonstrated functional connectivity alterations of specific regions within and/or between the default mode network, the salience network, and the central executive network. In particular functional connectivity alterations within the default mode network are also observed in patients suffering from sleep disturbance.

Treating a patient suffering from BD, including a treatment resistant form of the disorder, and associated sleep disturbance with 5-MeO-DMT or a pharmaceutically acceptable salt thereof reduces or eliminates the sleep disturbance and leads to an improvement of BD.

The reduction or elimination of sleep disturbance in a patient suffering from BD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from BD occurs not later than about 24 hours after the last administration of 5-MeO-DMT or a pharmaceutically acceptable salt thereof. The reduction or elimination of sleep disturbance in a patient suffering from BD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from BD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from BD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from BD, as reflected by a reduction in the CGI-S score, occurs not later than about 24 hours after the last administration of 5-MeO-DMT or a pharmaceutically acceptable salt thereof. An improvement in sleep disturbance in a patient suffering from BD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from BD, as reflected by a reduction in the CGI-S score, is observed on day 7 after the last administration of 5-MeO-DMT or a pharmaceutically acceptable salt thereof and preferably persists until at least 14 days, more preferably until at least 28 days.

The reduction or elimination of sleep disturbance in a patient suffering from BD may be reflected by an improvement at least in the score of the BDRS item sleep disturbance on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from BD as reflected by an improvement in the score of the BDRS item sleep disturbance occurs not later than about 24 hours after the last administration of 5-MeO-DMT or a pharmaceutically acceptable salt thereof. The reduction or elimination of sleep disturbance in a patient suffering from BD as reflected by an improvement in the score of the BDRS item sleep disturbance preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from BD as reflected by an improvement in the score of the BDRS item sleep disturbance, is observed on day 7 after the last administration of 5-MeO-DMT or a pharmaceutically acceptable salt thereof and preferably persists until at least 14 days, more preferably until at least 28 days.

The reduction or elimination of sleep disturbance in a patient suffering from BD, in particular of reduced sleep, is reflected by at least an improvement in the score of the MADRS item reduced sleep on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from BD, in particular of reduced sleep, as reflected by an improvement in the score of the MADRS item reduced sleep occurs not later than about 24 hours after the last administration of 5-MeO-DMT or a pharmaceutically acceptable salt thereof. The reduction or elimination of sleep disturbance in a patient suffering from BD, in particular of reduced sleep, as reflected by an improvement in the score of the MADRS item reduced sleep preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from BD, in particular of reduced sleep, as reflected by an improvement in the score of the MADRS item reduced sleep, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from BD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from BD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from BD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from BD, as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

The reduction or elimination of sleep disturbance in a patient suffering from BD, in particular of increase in total sleep time, is reflected by at least an improvement in the score of the BDRS item hypersomnia on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from BD, in particular of increase in total sleep time, as reflected by an improvement in the score of the BDRS item hypersomnia occurs not later than about 24 hours after the last administration of 5-MeO-DMT or a pharmaceutically acceptable salt thereof. The reduction or elimination of sleep disturbance in a patient suffering from BD, in particular of increase in total sleep time, as reflected by an improvement in the score of the BDRS item hypersomnia preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from BD, in particular of increase in total sleep time, as reflected by an improvement in the score of the BDRS item hypersomnia, is observed on day 7 after the last administration of 5-MeO-DMT or a pharmaceutically acceptable salt thereof and preferably persists until at least 14 days, more preferably until at least 28 days.

As indicated above, sleep disturbance is closely linked to BD. An improvement in sleep disturbance will therefore also lead to an improvement of BD. Since sleep disturbance furthermore also affects other aspects of BD, the inventors conclude that the improvement in sleep disturbance will additionally contribute to an overall improvement of BD.

An improvement of the BD in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the BD in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the BD in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of BD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Anxiety Disorders

An Anxiety Disorder is a type of mental health condition. Symptoms include feelings of nervousness, panic and fear as well as sweating and a rapid heartbeat. Anxiety involves a complex cognitive, affective, physiological, and behavioural response system associated with preparation for anticipated events or circumstances perceived as threatening.

A patient suffering from an Anxiety Disorder may suffer from a treatment resistant form of the disorder.

Patients suffering from an Anxiety Disorder often experience sleep disturbance, in particular insomnia. Excess worry and fear make it difficult to fall asleep and stay asleep through the night.

Insomnia can worsen anxiety, leading to a negative cycle involving insomnia and anxiety.

The severity of an Anxiety Disorder can be assessed by using the Hamilton Anxiety Rating Scale (HAM-A) which consists of 14 items, each defined by a series of symptoms, and measures both psychic anxiety (mental agitation and psychological distress) and somatic anxiety (physical complaints related to anxiety). Item 4 is ‘Insomnia’, defined as ‘Difficulty in falling asleep, broken sleep, unsatisfying sleep and fatigue on waking, dreams, nightmares, night terrors.’ Each item is scored on a scale of 0 (not present) to 4 (severe), with a total score range of 0-56.

Sleep disturbance can also be assessed using, for instance, the Pittsburgh Sleep Quality Index (PSQI) global score.

Anxiety Disorders are associated with alterations in the functional connectivity of resting state networks. Anxiety Disorders show abnormalities in the default mode network (DMN) affecting the sense of self, the salience network (SN) controlling emotion/anxiety and the somatomotor network (SMN) responsible for bodily awareness. The resting state balance within and/or between each of these networks, e.g., SMN and SN, relative to the DMN may be abnormal in the different anxiety disorders.

Altered functional connectivity in affected networks is also associated with sleep disturbances, and anxiety disorders are associated with sleep disturbances.

Treating a patient suffering from an Anxiety Disorder, including a treatment resistant form of the disorder, and associated sleep disturbance with 5-MeO-DMT or a pharmaceutically acceptable salt thereof reduces or eliminates the sleep disturbance and leads to an improvement of the Anxiety Disorder.

The reduction or elimination of sleep disturbance in a patient suffering from an Anxiety Disorder is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from an Anxiety Disorder occurs not later than about 24 hours after the last administration of 5-MeO-DMT or a pharmaceutically acceptable salt thereof. The reduction or elimination of sleep disturbance in a patient suffering from an Anxiety Disorder preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from an Anxiety Disorder is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Anxiety Disorder, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Anxiety Disorder, as reflected by a reduction in the CGI-S score, occurs not later than about 24 hours after the last administration of 5-MeO-DMT or a pharmaceutically acceptable salt thereof. An improvement in sleep disturbance in a patient suffering from Anxiety Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from an Anxiety Disorder, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from an Anxiety Disorder is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from an Anxiety Disorder as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from an Anxiety Disorder as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from an Anxiety Disorder as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance occurs in patients with an Anxiety Disorder. An improvement in sleep disturbance will therefore also lead to an improvement of the Anxiety Disorder. Since sleep disturbance furthermore also affects other aspects of the Anxiety Disorder, the inventors conclude that the improvement in sleep disturbance will additionally contribute to an overall improvement of the Anxiety Disorder.

An improvement of the Anxiety Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Anxiety Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Anxiety Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of an Anxiety Disorder in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Separation Anxiety Disorder is characterized by an excessive anxiety regarding separation from home and/or from someone to whom the patient has a strong emotional attachment.

Situations of separation cause the patient significant distress, and they may have difficulty going to school or work due to the separation. Patients suffering from Separation Anxiety Disorder may also have excessive anxiety about unwelcome events happening to important people in their lives, such as family members.

A patient suffering from Separation Anxiety Disorder may suffer from a treatment resistant form of the disorder.

Separation Anxiety Disorder is associated with sleep disturbances. Sleep disturbance is even part of the diagnostic criteria of Separation Anxiety Disorder. Sleep disturbances in patients suffering from Separation Anxiety Disorder include nightmares, difficulty falling or staying asleep, early waking, and parasomnias.

The severity of Separation Anxiety Disorder can be assessed by using the Hamilton Anxiety Rating Scale (HAM-A), in which the assessment of sleep disturbances is integral part of the scale. This scale may further be used as an outcome measure of a treatment according to the invention, by administering the scale pre- and post-treatment.

Sleep disturbances in Separation Anxiety Disorder can also be assessed by the Pittsburgh Sleep Quality Index (PSQI).

Anxiety Disorders are associated with alterations in the functional connectivity of resting state networks. Anxiety Disorders show abnormalities in the default mode network (DMN) affecting the sense of self, the salience network (SN) controlling emotion/anxiety and the somatomotor network (SMN) responsible for bodily awareness. The resting state balance within and/or between each of these networks, e.g., SMN and SN, relative to the DMN may be abnormal in the different anxiety disorders.

Altered functional connectivity in affected networks is also associated with sleep disturbances, and Separation Anxiety Disorder is associated with sleep disturbances.

Treating a patient suffering from Separation Anxiety Disorder, including a treatment resistant form of the disorder, and associated sleep disturbance with 5-MeO-DMT or a pharmaceutically acceptable salt thereof reduces or eliminates the sleep disturbance and leads to an improvement of the Separation Anxiety Disorder.

The reduction or elimination of sleep disturbance in a patient suffering from Separation Anxiety Disorder is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Separation Anxiety Disorder occurs not later than about 24 hours after the last administration of 5-MeO-DMT or a pharmaceutically acceptable salt thereof. The reduction or elimination of sleep disturbance in a patient suffering from Separation Anxiety Disorder preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Separation Anxiety Disorder is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Separation Anxiety Disorder, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Separation Anxiety Disorder, as reflected by a reduction in the CGI-S score, occurs not later than about 24 hours after the last administration of 5-MeO-DMT or a pharmaceutically acceptable salt thereof. An improvement in sleep disturbance in a patient suffering from Separation Anxiety Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from an Separation Anxiety Disorder, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Separation Anxiety Disorder is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Separation Anxiety Disorder as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Separation Anxiety Disorder as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Separation Anxiety Disorder as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is a significant complaint by patients with Separation Anxiety Disorder. An improvement in sleep disturbance will therefore also lead to an improvement of the Separation Anxiety Disorder. Since sleep disturbance furthermore also affects other aspects of Separation Anxiety Disorder, the inventors conclude that the improvement in sleep disturbance, in particular the reduction or elimination of reduced sleep, will additionally contribute to an overall improvement of the Separation Anxiety Disorder.

An improvement of the Separation Anxiety Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Separation Anxiety Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Separation Anxiety Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Separation Anxiety Disorder in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Agoraphobia is a fear of situations or places that may cause feelings of panic, entrapment, helplessness, or embarrassment.

A patient suffering from agoraphobia may have difficulty leaving the house. The thought of leaving the house may cause considerable anxiety to the point of avoidance. Fears of crowds, traveling, elevators, movie theatres, malls, etc., might cause significant challenges.

Patients with agoraphobia may also have recurrent panic attacks.

A patient suffering from Agoraphobia may suffer from a treatment resistant form of the disorder.

Agoraphobia is associated with sleep disturbances, such as insomnia.

The severity of Agoraphobia can be assessed by using the Hamilton Anxiety Rating Scale (HAM-A), in which the assessment of sleep disturbances is integral part of the scale. This scale may further be used as an outcome measure of a treatment according to the invention, by administering the scale pre- and post-treatment.

Sleep disturbances in Agoraphobia can also be assessed by the Pittsburgh Sleep Quality Index (PSQI).

Functional magnetic resonance imaging in individuals suffering from Agoraphobia reveals alterations in functional connectivity within and/or between resting-state networks involved in Anxiety.

Treating a patient suffering from Agoraphobia, including a treatment resistant form of the disorder, and associated sleep disturbance with 5-MeO-DMT or a pharmaceutically acceptable salt thereof reduces or eliminates the sleep disturbance and leads to an improvement of the Agoraphobia.

The reduction or elimination of sleep disturbance in a patient suffering from Agoraphobia is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Agoraphobia occurs not later than about 24 hours after the last administration of 5-MeO-DMT or a pharmaceutically acceptable salt thereof. The reduction or elimination of sleep disturbance in a patient suffering from Agoraphobia preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Agoraphobia is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Agoraphobia, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Agoraphobia, as reflected by a reduction in the CGI-S score, occurs not later than about 24 hours after the last administration of 5-MeO-DMT or a pharmaceutically acceptable salt thereof. An improvement in sleep disturbance in a patient suffering from Agoraphobia, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Agoraphobia, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Agoraphobia is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Agoraphobia as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Agoraphobia as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Agoraphobia as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is a significant complaint by patients with Agoraphobia. An improvement in sleep disturbance will therefore also lead to an improvement of the Agoraphobia. Since sleep disturbance furthermore also affects other aspects of Agoraphobia, the inventors conclude that the improvement in sleep disturbance, in particular the reduction or elimination of reduced sleep, will additionally contribute to an overall improvement of the Agoraphobia.

An improvement of the Agoraphobia in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Agoraphobia in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Agoraphobia in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Agoraphobia in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Generalized Anxiety Disorder (GAD) is characterized by persistent and excessive, difficult to control worry about a wide range of situations and issues. Patients suffering from GAD may anticipate disaster and may be overly concerned about money, health, family, work, or other issues.

Generalized anxiety disorder is diagnosed when an individual experiences persistent worry about everyday challenges out of proportion to the perceived threat. Patients with GAD usually experience excessive fear that can last months to years.

GAD interferes with social, occupational, or other important areas of functioning.

The patient suffering from GAD may suffer from a treatment resistant form of the disorder.

A key feature of GAD is sleep disturbance, in particular including difficulty falling or staying asleep, or restless unsatisfying sleep. Sleep disturbance is associated with significant disability in GAD. In the DSM-5 criteria, sleep disturbance is one of 6 symptoms of which at least 3 need to be present for a certain period of time to satisfy a diagnosis of GAD.

The severity of Generalized Anxiety Disorder can be assessed using the Hamilton Anxiety Rating Scale (HAM-A), in which the assessment of sleep disturbances is integral part of the scale. This scale may further be used as an outcome measure of a treatment according to the invention, by administering the scale pre- and post-treatment.

Sleep disturbances in Generalized Anxiety Disorder can also be assessed by the Pittsburgh Sleep Quality Index (PSQI).

Patients with GAD show also altered functional connectivity, especially within the default mode network. Altered functional connectivity in affected networks is also associated with sleep disturbances.

Treating a patient suffering from GAD, including a treatment resistant form of the disorder, and associated sleep disturbance with 5-MeO-DMT or a pharmaceutically acceptable salt thereof reduces or eliminates the sleep disturbance and leads to an improvement of the GAD.

The reduction or elimination of sleep disturbance in a patient suffering from GAD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from GAD occurs not later than about 24 hours after the last administration of 5-MeO-DMT or a pharmaceutically acceptable salt thereof. The reduction or elimination of sleep disturbance in a patient suffering from GAD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from GAD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from GAD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from GAD, as reflected by a reduction in the CGI-S score, occurs not later than about 24 hours after the last administration of 5-MeO-DMT or a pharmaceutically acceptable salt thereof. An improvement in sleep disturbance in a patient suffering from GAD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from GAD, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from GAD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from GAD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from GAD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from GAD as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is one of the most common primary complaints by patients with GAD. An improvement in sleep disturbance will therefore also lead to an improvement of the GAD. Since sleep disturbance furthermore also affects other aspects of GAD, the inventors conclude that the improvement in sleep disturbance, in particular the reduction or elimination of reduced sleep, will additionally contribute to an overall improvement of the GAD.

An improvement of the GAD in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the GAD in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the GAD in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of GAD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Social Anxiety Disorder (SAD), also called social phobia, is one of the most common types of anxiety. SAD is characterized by intense anxiety or fear of being judged, negatively evaluated, or rejected in a social or performance situation. This often leads to avoidance of the social situation and can cause impairments in school, work, or relationships.

The patient suffering from SAD may suffer from a treatment resistant form of the disorder.

Sleep disturbances are very common in patients with social anxiety disorder given a bidirectional relationship between sleep disturbances (mainly insomnia) and anxiety.

The severity of Social Anxiety Disorder can be assessed by using the Hamilton Anxiety Rating Scale (HAM-A), in which the assessment of sleep disturbances is integral part of the scale. This scale may further be used as an outcome measure of a treatment according to the invention, by administering the scale pre- and post-treatment.

Sleep disturbances in Social Anxiety Disorder can also be assessed by the Pittsburgh Sleep Quality Index (PSQI).

Insomnia is reflected by altered functional connectivity within and/or between resting state networks, such as the default mode network and salience network, networks in which altered functional connectivity is observed in patients suffering from SAD.

Treating a patient suffering from SAD, including a treatment resistant form of the disorder, and associated sleep disturbance with 5-MeO-DMT or a pharmaceutically acceptable salt thereof reduces or eliminates the sleep disturbance and leads to an improvement of the SAD.

The reduction or elimination of sleep disturbance in a patient suffering from SAD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from SAD occurs not later than about 24 hours after the last administration of 5-MeO-DMT or a pharmaceutically acceptable salt thereof. The reduction or elimination of sleep disturbance in a patient suffering from SAD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from SAD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from SAD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from SAD, as reflected by a reduction in the CGI-S score, occurs not later than about 24 hours after the last administration of 5-MeO-DMT or a pharmaceutically acceptable salt thereof. An improvement in sleep disturbance in a patient suffering from SAD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from SAD, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from SAD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from SAD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from SAD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from SAD as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is a particularly important symptom in patients with SAD. An improvement in sleep disturbance will therefore also lead to an improvement of the SAD. Since sleep disturbance furthermore also affects other aspects of SAD, the inventors conclude that the improvement in sleep disturbance, in particular the reduction or elimination of reduced sleep, will additionally contribute to an overall improvement of the SAD.

An improvement of the SAD in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the SAD in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the SAD in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of SAD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Patients suffering from Panic Disorder experience spontaneous panic attacks with an abrupt onset of intense fear or discomfort that reaches a peak within minutes.

The panic attacks feature many somaticized symptoms of anxiety including sweating, trembling, shaking, headache, palpitations, shortness of breath, chest pain, abdominal pain, and nausea.

Furthermore, the disorder can often feature anxiety of future panic attacks. Patients may be very preoccupied with the fear of a recurring attack.

The patient suffering from Panic Disorder may suffer from a treatment resistant form of the disorder.

Sleep disturbances (particularly insomnia and hypersomnia) are very common in patients with panic disorder.

Patients with Panic Disorder may also experience nocturnal panic attacks which are distinguished from night terrors. Nocturnal panic attacks can occur without a trigger and often result in difficulty falling back asleep.

The severity of Panic Disorder can be assessed by using the Hamilton Anxiety Rating Scale (HAM-A), in which the assessment of sleep disturbances is integral part of the scale. This scale may further be used as an outcome measure of a treatment according to the invention, by administering the scale pre- and post-treatment.

Sleep disturbances in Panic Disorder can also be assessed by the Pittsburgh Sleep Quality Index (PSQI).

Patients with panic disorder show altered functional connectivity within and/or between the default mode network and somatomotor network. Altered functional connectivity within and/or between resting state networks, in particular in the default mode network, also characterises insomnia and hypersomnia.

Treating a patient suffering from Panic Disorder, including a treatment resistant form of the disorder, and associated sleep disturbance with 5-MeO-DMT or a pharmaceutically acceptable salt thereof reduces or eliminates the sleep disturbance and leads to an improvement of the Panic Disorder.

The reduction or elimination of sleep disturbance in a patient suffering from Panic Disorder is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Panic Disorder occurs not later than about 24 hours after the last administration of 5-MeO-DMT or a pharmaceutically acceptable salt thereof. The reduction or elimination of sleep disturbance in a patient suffering from Panic Disorder preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Panic Disorder is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Panic Disorder, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Panic Disorder, as reflected by a reduction in the CGI-S score, occurs not later than about 24 hours after the last administration of 5-MeO-DMT or a pharmaceutically acceptable salt thereof. An improvement in sleep disturbance in a patient suffering from Panic Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Panic Disorder, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Panic Disorder is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Panic Disorder as reflected by an improvement in the PSQI global score occurs, in particular by a decrease to 5 or below, not later than about 24 hours after the last administration of 5-MeO-DMT or a pharmaceutically acceptable salt thereof. The reduction or elimination of sleep disturbance in a patient suffering from Panic Disorder as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Panic Disorder as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is very common in patients with Panic Disorder. An improvement in sleep disturbance will therefore also lead to an improvement of the Panic Disorder. Since sleep disturbance furthermore also affects other aspects of Panic Disorder, the inventors conclude that the improvement in sleep disturbance, in particular the reduction or elimination of insomnia and/or hypersomnia, will additionally contribute to an overall improvement of the Panic Disorder.

An improvement of the Panic Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Panic Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Panic Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Panic Disorder in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

A Phobia is an anxiety disorder defined by a persistent and excessive fear of an object or situation. A patient suffering from phobia experiences extreme anxiety when anticipating exposure or being exposed to a feared stimulus. There are animal type (spiders, snakes, dogs) phobias, natural environment type (tornadoes, heights, water, fire) phobias, blood injection type (needles, medical procedures) phobias, situational type (flying on an airplane, enclosed spaces) phobias and other type phobias (phobias that do not fit into one the previous categories).

Phobias typically result in a rapid onset of fear and are usually present for more than six months. Patients make great efforts to avoid the feared stimulus. Fear and avoidance cause significant distress and/or impairment in occupational, academic, or social functioning.

The patient suffering from a Phobia may suffer from a treatment resistant form of the disorder.

There is evidence of an increased likelihood of sleep disturbance in patients with Phobias.

The severity of Phobia can be assessed by using the Hamilton Anxiety Rating Scale (HAM-A), in which the assessment of sleep disturbances is integral part of the scale. This scale may further be used as an outcome measure of a treatment according to the invention, by administering the scale pre- and post-treatment.

Sleep disturbances in Phobia can also be assessed by the Pittsburgh Sleep Quality Index (PSQI).

Phobias are also reflected in altered functional connectivity of resting state networks, for instance, networks involving Amygdala and Insula, such as for example the salience network which also plays a role in insomnia.

Treating a patient suffering from a Phobia, including a treatment resistant form of the disorder, and associated sleep disturbance with 5-MeO-DMT or a pharmaceutically acceptable salt thereof reduces or eliminates the sleep disturbance and leads to an improvement of the Phobia.

The reduction or elimination of sleep disturbance in a patient suffering from a Phobia is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from a Phobia occurs not later than about 24 hours after the last administration of 5-MeO-DMT or a pharmaceutically acceptable salt thereof. The reduction or elimination of sleep disturbance in a patient suffering from a Phobia preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from a Phobia is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Phobia, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Phobia, as reflected by a reduction in the CGI-S score, occurs not later than about 24 hours after the last administration of 5-MeO-DMT or a pharmaceutically acceptable salt thereof. An improvement in sleep disturbance in a patient suffering from Phobia, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from a Phobia, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from a Phobia is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from a Phobia as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from a Phobia as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from a Phobia as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, there is an increased likelihood of sleep disturbance in patients with Phobias. An improvement in sleep disturbance will therefore also lead to an improvement of the Phobia. Since sleep disturbance furthermore also affects other aspects of Phobias, the inventors conclude that the improvement in sleep disturbance, in particular the reduction or elimination of reduced sleep, will additionally contribute to an overall improvement of the Phobia.

An improvement of the Phobia in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Phobia in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Phobia in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of a Phobia in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Substance/Medication Induced Anxiety Disorder is an anxiety disorder in which anxiety or panic occurs after using alcohol, a drug of abuse, or a medication or after a toxin exposure. Substance/medication-induced anxiety disorder leads to prominent symptoms of panic or anxiety and can occur during the intoxication or withdrawal phases of using a substance or medication.

The disturbance causes clinically significant distress or impairment in social, occupational, or other important areas of functioning.

While taking substances or medication or within a short time thereafter, individuals suffering from Substance/Medication Induced Anxiety Disorder may feel nervous and worried, experience symptoms of negative thinking, may have trouble concentrating or remembering things, may have fear of losing control or insanity or death, may lose weight due to gastrointestinal problems, may have chills, hot flashes, sweating, shaking, numbness, or a pounding heartbeat, trouble breathing, trouble swallowing, or chest pain.

The patient suffering from Substance/Medication Induced Anxiety Disorder may suffer from a treatment resistant form of the disorder.

Substance/Medication Induced Anxiety Disorder is also accompanied by sleep disturbances. Patients are having trouble falling asleep or waking up often during the night.

The severity of Substance/Medication Induced Anxiety Disorder can be assessed by using the Hamilton Anxiety Rating Scale (HAM-A), in which the assessment of sleep disturbances is integral part of the scale. This scale may further be used as an outcome measure of a treatment according to the invention, by administering the scale pre- and post-treatment.

Sleep disturbances in Substance/Medication Induced Anxiety Disorder can also be assessed by the Pittsburgh Sleep Quality Index (PSQI).

Functional magnetic resonance imaging in individuals suffering from Substance/Medication Induced Anxiety Disorder reveals alterations in functional connectivity within and/or between resting-state networks involved in anxiety.

Treating a patient suffering from Substance/Medication Induced Anxiety Disorder, including a treatment resistant form of the disorder, and associated sleep disturbance with 5-MeO-DMT or a pharmaceutically acceptable salt thereof reduces or eliminates the sleep disturbance and leads to an improvement of the Substance/Medication Induced Anxiety Disorder.

The reduction or elimination of sleep disturbance in a patient suffering from Substance/Medication Induced Anxiety Disorder is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Substance/Medication Induced Anxiety Disorder occurs not later than about 24 hours after the last administration of 5-MeO-DMT or a pharmaceutically acceptable salt thereof. The reduction or elimination of sleep disturbance in a patient suffering from Substance/Medication Induced Anxiety Disorder preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Substance/Medication Induced Anxiety Disorder is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Substance/Medication Induced Anxiety Disorder, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Substance/Medication Induced Anxiety Disorder, as reflected by a reduction in the CGI-S score, occurs not later than about 24 hours after the last administration of 5-MeO-DMT or a pharmaceutically acceptable salt thereof. An improvement in sleep disturbance in a patient suffering from Substance/Medication Induced Anxiety Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Substance/Medication Induced Anxiety Disorder, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Substance/Medication Induced Anxiety Disorder is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Substance/Medication Induced Anxiety Disorder as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Substance/Medication Induced Anxiety Disorder as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Substance/Medication Induced Anxiety Disorder as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is a significant complaint by patients with Substance/Medication Induced Anxiety Disorder. An improvement in sleep disturbance will therefore also lead to an improvement of the Substance/Medication Induced Anxiety Disorder. Since sleep disturbance furthermore also affects other aspects of Substance/Medication Induced Anxiety Disorder, the inventors conclude that the improvement in sleep disturbance, in particular the reduction or elimination of reduced sleep, will additionally contribute to an overall improvement of the Substance/Medication Induced Anxiety Disorder.

An improvement of the Substance/Medication Induced Anxiety Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Substance/Medication Induced Anxiety Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Substance/Medication Induced Anxiety Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Substance/Medication Induced Anxiety Disorder in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Somatic Symptom Disorder

Somatic Symptom Disorder is a mental disorder diagnosed when a person has a significant focus on physical symptoms, such as pain, weakness, or shortness of breath to a level that results in major distress and/or problems functioning and/or cause disruption in daily life. Feelings and behaviours related to the illness are excessive or out of proportion.

Health-related quality of life is often impaired, both physically and mentally. In severe Somatic Symptom Disorder, the impairment is marked, and when persistent, the disorder can lead to invalidism.

Patients with Somatic Symptom Disorder may report sleep disturbance (i.e. insomnia, patient's dissatisfaction regarding the quality, timing, and amount of sleep), with resulting daytime distress and impairment.

Sleep disturbance has the potential of creating a veritable vicious circle between the somatic symptom and sleep disturbance.

For instance, insomnia is correlated with the presence of somatic symptoms and the severity of insomnia is correlated with the severity of somatic symptoms. However, this effect is not limited to insomnia, as changes in sleep-wake cycle can themselves be associated to the occurrence of somatic symptoms.

Somatic symptom disorder can be assessed by the DSM-5 Level 2—Somatic Symptom—Adult measure. This measure comprises 15 somatic symptoms. Respondents are asked to rate the severity of the individual's somatic symptoms during the past 7 days. Sleep disturbances are analysed by scoring “Feeling tired or having low energy” and “Trouble sleeping”. Scoring ranges from “0” (“Not bothered at all”), “1” (“Bothered a little”) to “2” (“Bothered a lot”). The total score can range from 0 to 30, with higher scores indicating greater severity of somatic symptoms. A cut-off value of 5, 10 and 15 indicates low, medium, high somatic symptom severity, respectively.

Moreover, sleep disturbance in individuals suffering from Somatic Symptom Disorder can be assessed using the Pittsburgh Sleep Quality Index.

Brain functional connectivity analysis reveals alterations within and/or between resting state networks in patients with Somatic Symptom Disorder in comparison to healthy controls. Alterations are identified within and/or between default mode network (DMN), salience network, dorsal attention network (DAN) and sensorimotor network. Somatic Symptom Disorder may be associated with alterations of sensory-discriminative processing of somatic symptoms, which is influenced by affective processing.

Treating a patient suffering from Somatic Symptom Disorder and associated sleep disturbance with 5-MeO-DMT or a pharmaceutically acceptable salt thereof reduces or eliminates the sleep disturbance and leads to an improvement of the Somatic Symptom Disorder.

The reduction or elimination of sleep disturbance in a patient suffering from Somatic Symptom Disorder is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Somatic Symptom Disorder occurs not later than about 24 hours after the last administration of 5-MeO-DMT or a pharmaceutically acceptable salt thereof. The reduction or elimination of sleep disturbance in a patient suffering from Somatic Symptom Disorder preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Somatic Symptom Disorder is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Somatic Symptom Disorder, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Somatic Symptom Disorder, as reflected by a reduction in the CGI-S score, occurs not later than about 24 hours after the last administration of 5-MeO-DMT or a pharmaceutically acceptable salt thereof. An improvement in sleep disturbance in a patient suffering from Somatic Symptom Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Somatic Symptom Disorder, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Somatic Symptom Disorder is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Somatic Symptom Disorder as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Somatic Symptom Disorder as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Somatic Symptom Disorder as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is common in patients with Somatic Symptom Disorder. An improvement in sleep disturbance will therefore also lead to an improvement of the Somatic Symptom Disorder. Since sleep disturbance furthermore also affects other aspects of Somatic Symptom Disorder, the inventors conclude that the improvement in sleep disturbance, in particular the reduction or elimination of reduced sleep, will additionally contribute to an overall improvement of the Somatic Symptom Disorder.

An improvement of the Somatic Symptom Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Somatic Symptom Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Somatic Symptom Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Somatic Symptom Disorder in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Obsessive Compulsive and Related Disorders

Obsessive Compulsive Disorder (OCD) is a mental illness that causes repeated unwanted thoughts or sensations (obsessions) or the urge to do something over and over again (compulsions). Patients may suffer from both obsessions and compulsions.

The patient suffering from OCD may suffer from a treatment resistant form of the disorder.

Sleep disturbance is a common feature in patients suffering from OCD. There is a correlation between the presence of sleep disturbance and the severity of OCD.

Sleep disturbance is associated with treatment resistance of OCD and as such addressing the symptom will provide for enhanced treatment efficacy.

Neuroimaging studies using functional magnetic resonance imaging of patients suffering from OCD show functional connectivity alterations within and/or between frontoparietal network, salience network, and default mode network. Altered functional connectivity in affected networks, in particular the default mode network, is also associated with sleep disturbances.

Treating a patient suffering from OCD, including a treatment resistant form of the disorder, and associated sleep disturbance with 5-MeO-DMT or a pharmaceutically acceptable salt thereof reduces or eliminates the sleep disturbance and leads to an improvement of the OCD.

The reduction or elimination of sleep disturbance in a patient suffering from OCD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from OCD occurs not later than about 24 hours after the last administration of 5-MeO-DMT or a pharmaceutically acceptable salt thereof. The reduction or elimination of sleep disturbance in a patient suffering from OCD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from OCD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from OCD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from OCD, as reflected by a reduction in the CGI-S score, occurs not later than about 24 hours after the last administration of 5-MeO-DMT or a pharmaceutically acceptable salt thereof. An improvement in sleep disturbance in a patient suffering from OCD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from OCD, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from OCD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from OCD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from OCD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from OCD as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is common in patients with OCD. An improvement in sleep disturbance will therefore also lead to an improvement of the OCD. Since sleep disturbance furthermore also affects other aspects of OCD, the inventors conclude that the improvement in sleep disturbance, in particular the reduction or elimination of reduced sleep, will additionally contribute to an overall improvement of the OCD.

An improvement of the OCD in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the OCD in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the OCD in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of OCD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Patients suffering from Body Dysmorphic Disorder (BDD) misperceive defects in their appearance, disrupting their ability to function in their daily lives, with disturbing preoccupations, ritualistic behaviours, and emotional distress.

The patient suffering from BDD may suffer from a treatment resistant form of the disorder.

Sleep disturbance, in particular insomnia, is a common feature in patients suffering from BDD, and is associated with more severe psychopathology. Patients suffering from BDD and sleep disturbance show higher self-reported BDD symptom severity, more depressive symptoms, and more functional impairment in daily activities.

Functional magnetic resonance imaging of patients suffering from BDD reveals alterations within and/or between certain brain areas located in the default mode network, the dorsal attention network and the salience network. Altered functional connectivity in affected networks, in particular the default mode network, is also associated with sleep disturbances.

Treating a patient suffering from BDD, including a treatment resistant form of the disorder, and associated sleep disturbance with 5-MeO-DMT or a pharmaceutically acceptable salt thereof reduces or eliminates the sleep disturbance and leads to an improvement of the BDD.

The reduction or elimination of sleep disturbance in a patient suffering from BDD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from BDD occurs not later than about 24 hours after the last administration of 5-MeO-DMT or a pharmaceutically acceptable salt thereof. The reduction or elimination of sleep disturbance in a patient suffering from BDD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from BDD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from BDD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from BDD, as reflected by a reduction in the CGI-S score, occurs not later than about 24 hours after the last administration of 5-MeO-DMT or a pharmaceutically acceptable salt thereof. An improvement in sleep disturbance in a patient suffering from BDD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from BDD, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from BDD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from BDD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from BDD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from BDD as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is common in patients with BDD. An improvement in sleep disturbance will therefore also lead to an improvement of the BDD. Since sleep disturbance furthermore also affects other aspects of BDD, the inventors conclude that the improvement in sleep disturbance, in particular the reduction or elimination of reduced sleep, will additionally contribute to an overall improvement of the BDD.

An improvement of the BDD in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the BDD in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the BDD in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of BDD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Post-Traumatic Stress Disorder (PTSD)

Post-Traumatic Stress Disorder (PTSD) is a mental health condition that can develop based on a terrifying event—either experienced or witnessed by the patient. Symptoms may include flashbacks, nightmares and severe anxiety, as well as uncontrollable thoughts about the event.

The patient suffering from PTSD may suffer from a treatment resistant form of the disorder.

Sleep disturbance has been associated with PTSD, with estimates of 70-90% of patients reporting at least one type of sleep disturbance (insomnia, difficulty falling or staying asleep, parasomnia, such as nightmares).

Sleep disturbance in form of difficulty falling asleep, staying asleep or restless sleep, is one of 6 features of altered arousal of which at least 2 are needed for a diagnosis of PTSD according to the DSM 5 criteria.

Sleep disturbances in PTSD can be assessed by self-administered questionnaires, such as the PTSD Symptom Scale or the Trauma Screening Questionnaire (TSQ).

The PTSD Symptom Scale in the self-report version is a 17-item self-reported questionnaire to assess symptoms of posttraumatic stress disorder. Sleep disturbances are evaluated in form of the occurrence of bad dreams or nightmares or having trouble falling or staying asleep. Respondents are asked to indicate how frequently they have experienced certain sleep difficulties over the past two weeks or another appropriate recall window using a Likert-type scale ranging from 0 (not at all or only one time) to 3 (almost always or five or more times per week). A score of 13 or higher indicates the likelihood of PTSD.

In the Trauma Screening questionnaire, sleep (bad dreams or difficulties in falling or staying asleep) is analysed in two out of 10 items, which require a yes or no answer. Six or more positive responses are indicative for a risk of having PTSD.

Analysis of resting state functional magnetic resonance imaging in patients suffering from PTSD reveals alterations within and/or between regions located in the default mode network and salience network.

Treating a patient suffering from PTSD, including a treatment resistant form of the disorder, and associated sleep disturbance with 5-MeO-DMT or a pharmaceutically acceptable salt thereof reduces or eliminates the sleep disturbance and leads to an improvement of the PTSD.

The reduction or elimination of sleep disturbance in a patient suffering from PTSD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from PTSD occurs not later than about 24 hours after the last administration of 5-MeO-DMT or a pharmaceutically acceptable salt thereof. The reduction or elimination of sleep disturbance in a patient suffering from PTSD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from PTSD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from PTSD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from PTSD, as reflected by a reduction in the CGI-S score, occurs not later than about 24 hours after the last administration of 5-MeO-DMT or a pharmaceutically acceptable salt thereof. An improvement in sleep disturbance in a patient suffering from PTSD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from PTSD, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from PTSD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from PTSD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from PTSD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from PTSD as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is an important aspect of PTSD. An improvement in sleep disturbance will therefore also lead to an improvement of the PTSD. Since sleep disturbance furthermore also affects other aspects of PTSD, the inventors conclude that the improvement in sleep disturbance, in particular the reduction or elimination of reduced sleep, will additionally contribute to an overall improvement of the PTSD.

An improvement of the PTSD in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the PTSD in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the PTSD in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of PTSD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Pain Disorders

Sleep disturbance occurs in patients suffering from pain and has been associated with Chronic Pain.

Chronic Pain, also referred to as persistent pain, is long standing pain that persists beyond the usual recovery period, for instance, after an injury or operation, despite medication or treatment. Patients may also suffer from Chronic Pain without any apparent cause, such as a history of an injury or operation.

The prevalence of insomnia defined as dissatisfaction with sleep quantity or quality, difficulty initiating, maintaining, or early waking within the Chronic Pain population is nearly double that of the general population, estimated at 24-32%.

When sleep disturbance is defined as through the use of polysomnography, actinography or self-reported measures, the estimate of prevalence within Chronic Pain patients climbs to 40-80%.

The relationship between sleep disturbance and Chronic Pain is thought to be bidirectional, however insomnia may later develop into an independent condition that would require direct targeted treatment. Other conditions associated with sleep disturbance such as depression and anxiety are highly prevalent within the Chronic Pain population.

The relevance of sleep disturbances for Chronic Pain is also reflected by, for instance, the Brief Pain Inventory-Short Form (BPI-sf), a 9-item self-administered questionnaire, that is used to evaluate the severity of a patient's pain and the impact of this pain on the patient's daily functioning. One item of the BPI-sf is sleep. The questionnaire addresses the interference of pain with sleep during the past 24 hours. Thus, sleep disturbance is a relevant aspect of Chronic Pain.

Chronic Pain patients display brain alterations regarding brain function and structure. These changes are related to the persistence of pain, long after the initial nociceptive input has disappeared. Resting state functional magnetic resonance imaging reveals alterations in distinct regions within and/or between the default mode network, the somatomotor/sensorimotor network, and the salience network.

Treating a patient suffering from Chronic Pain and associated sleep disturbance with 5-MeO-DMT or a pharmaceutically acceptable salt thereof reduces or eliminates the sleep disturbance and leads to an improvement of the Chronic Pain.

The reduction or elimination of sleep disturbance in a patient suffering from Chronic Pain is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Chronic Pain occurs not later than about 24 hours after the last administration of 5-MeO-DMT or a pharmaceutically acceptable salt thereof. The reduction or elimination of sleep disturbance in a patient suffering from Chronic Pain preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Chronic Pain is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Chronic Pain, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Chronic Pain, as reflected by a reduction in the CGI-S score, occurs not later than about 24 hours after the last administration of 5-MeO-DMT or a pharmaceutically acceptable salt thereof. An improvement in sleep disturbance in a patient suffering from Chronic Pain, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Chronic Pain, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Chronic Pain is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Chronic Pain as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Chronic Pain as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Chronic Pain as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is an important disease aspect in patients with Chronic Pain. An improvement in sleep disturbance will therefore also lead to an improvement of the Chronic Pain. Since sleep disturbance furthermore also affects other aspects of Chronic Pain, the inventors conclude that the improvement in sleep disturbance, in particular the reduction or elimination of reduced sleep, will additionally contribute to an overall improvement of the Chronic Pain.

An improvement of the Chronic Pain in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Chronic Pain in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Chronic Pain in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Chronic Pain in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Fibromyalgia is a chronic disorder that is characterized by widespread musculoskeletal pain throughout the body or at multiple sites, accompanied by fatigue, sleep disturbances, memory and mood issues. Patients may also encounter muscle and joint stiffness, tenderness to touch, numbness or tingling in the arms and legs, problems with concentrating, thinking clearly, and memory (sometimes called “fibro fog”), heightened sensitivity to light, noise, odors, and temperature, or digestive issues, such as bloating or constipation.

Studies show that Fibromyalgia patients have a heightened sensitivity to pain, so they feel pain when others do not.

Sleep problems and fatigue are a common symptom of Fibromyalgia. Insomnia, non-restorative sleep, and fatigue are commonly used as markers for a fibromyalgia diagnosis. Fibromyalgia patients may also suffer from restless leg syndrome.

Sleep disturbances in Fibromyalgia can be assessed using the Pittsburgh Sleep Quality Index (PSQI).

There is a bidirectional relationship between Fibromyalgia and sleep disturbance. Pain symptoms can prevent patients from getting enough rest, and lack of sleep can decrease the pain threshold and exacerbate feelings of pain and tenderness.

The interplay of pain, fatigue, and poor sleep quality often interferes with a patient's ability to function at home or on the job.

Previous treatment of Fibromyalgia is symptomatic.

Brain imaging studies and other research have uncovered evidence of altered signaling in neural pathways that transmit and receive pain in people with Fibromyalgia. These changes may also contribute to the fatigue, sleep disturbances, and cognitive problems that many people with the disorder experience.

Resting-state functional magnetic resonance imaging of patients with Fibromyalgia shows altered functional connectivity within and/or between the DMN and executive attention network and between the DMN and the insular cortex, a brain region known to process evoked pain.

Treating a patient suffering from Fibromyalgia and associated sleep disturbance with 5-MeO-DMT or a pharmaceutically acceptable salt thereof reduces or eliminates the sleep disturbance and leads to an improvement of the Fibromyalgia.

The reduction or elimination of sleep disturbance in a patient suffering from Fibromyalgia is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Fibromyalgia occurs not later than about 24 hours after the last administration of 5-MeO-DMT or a pharmaceutically acceptable salt thereof. The reduction or elimination of sleep disturbance in a patient suffering from Fibromyalgia preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Fibromyalgia is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Fibromyalgia, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Fibromyalgia, as reflected by a reduction in the CGI-S score, occurs not later than about 24 hours after the last administration of 5-MeO-DMT or a pharmaceutically acceptable salt thereof. An improvement in sleep disturbance in a patient suffering from Fibromyalgia, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Fibromyalgia, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Fibromyalgia is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Fibromyalgia as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Fibromyalgia as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Fibromyalgia as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is one of the most common primary complaints by patients with Fibromyalgia. An improvement in sleep disturbance will therefore also lead to an improvement of the Fibromyalgia. Since sleep disturbance furthermore also affects other aspects of Fibromyalgia, the inventors conclude that the improvement in sleep disturbance, in particular the reduction or elimination of reduced sleep, will additionally contribute to an overall improvement of the Fibromyalgia.

An improvement of the Fibromyalgia in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Fibromyalgia in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Fibromyalgia in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Fibromyalgia in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Migraine is a headache that can cause severe throbbing pain or a pulsing sensation, usually on one side of the head. Migraine is often accompanied by nausea, vomiting, and extreme sensitivity to light and sound. Migraine attacks can last for hours to days, and the pain can be so severe that it interferes with daily activities.

In some patients, a symptom known as an aura occurs before or with the headache. This symptom can include visual disturbances, such as flashes of light or blind spots, or other disturbances, such as tingling on one side of the face or in an arm or leg and difficulty speaking.

The most common sleep disturbance in patients suffering from migraine is insomnia. This includes difficulty falling or staying asleep, early morning awakenings and non-refreshing sleep. Insomnia impairs daytime functions, which results in fatigue, poor attention and concentration, and loss of motivation.

The presence of sleep disturbance is associated with more frequent and severe migraine.

The Pittsburgh Sleep Quality Index (PSQI) is a common self-reported sleep quality questionnaire used to assess sleep quality over the past month and has been used in many studies to assess poor sleep quality in migraine patients.

Headache disorders are associated with atypical functional connectivity of regions associated with pain processing as well as atypical functional connectivity within and/or between multiple core resting state networks, including the salience and the default mode network.

In patients suffering from migraine, resting state network analysis shows differences to healthy controls. Studies during the migraine attacks reveal marked abnormalities in networks relevant for mediating cognitive, attentional, somatosensory and emotional components of pain.

Treating a patient suffering from Migraine and associated sleep disturbance with 5-MeO-DMT or a pharmaceutically acceptable salt thereof reduces or eliminates the sleep disturbance and leads to an improvement of the Migraine.

The reduction or elimination of sleep disturbance in a patient suffering from Migraine is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Migraine occurs not later than about 24 hours after the last administration of 5-MeO-DMT or a pharmaceutically acceptable salt thereof. The reduction or elimination of sleep disturbance in a patient suffering from Migraine preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Migraine is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Migraine, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Migraine, as reflected by a reduction in the CGI-S score, occurs not later than about 24 hours after the last administration of 5-MeO-DMT or a pharmaceutically acceptable salt thereof. An improvement in sleep disturbance in a patient suffering from Migraine, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Migraine, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Migraine is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Migraine as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Migraine as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

As indicated above, sleep disturbance is one of the most common primary complaints by patients with Migraine. An improvement in sleep disturbance will therefore also lead to an improvement of the Migraine. Since sleep disturbance furthermore also affects other aspects of Migraine, the inventors conclude that the improvement in sleep disturbance, in particular the reduction or elimination of reduced sleep, will additionally contribute to an overall improvement of the Migraine.

In an embodiment, a reduction or elimination of sleep disturbance in a patient suffering from Migraine as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

An improvement of the Migraine in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Migraine in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Migraine in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Migraine in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Mental and Behavioural Disorders Due to Psychoactive Substance Use

Sleep disturbance occurs in patients suffering from certain mental and behavioural disorders due to psychoactive substance use.

A Substance Use Disorder (SUD) is a mental disorder that affects a person's behaviour, leading to a person's inability to control their use of substances such as legal or illegal drugs, alcohol, or medications. Symptoms can range from moderate to severe, with addiction being the most severe form of SUDs.

Resting state functional connectivity (rsFC) was found to be altered not only in patients with sleep disturbances, but also in patients with Substance Use Disorders. In particular, deficits in cognitive control are associated with altered connectivity within and/or between resting state networks, such as the default mode network, the salience network, the central executive network, the limbic network and the reward network.

Substance/medication-associated sleep disorder is characterized by a severe change of sleeping patterns enough to warrant independent clinical attention and judged to be primarily caused by the pharmacological effects of a substance (i.e., a drug of abuse, a medication, toxin exposure).

Sleep disturbances can be the result of substance intoxication and/or withdrawal, such as alcohol, caffeine, cannabis, opioids, sedatives (hypnotics or anxiolytics), tobacco, stimulants (such as cocaine), or other substances.

Certain medications such as adrenergic agonists/antagonists, dopamine agonists/antagonists, cholinergic agonists/antagonists, serotonergic agonists/antagonists, antihistamines, or corticosteroids can also cause sleep disturbances.

Depending on the substance involved, one of four types of sleep disturbances is reported. Insomnia type and daytime sleepiness type are most common, while parasomnia-type is seen less often. The mixed type is noted when more than one type of sleep disturbance-related symptom is present, and none predominates.

As discontinuation/withdrawal states for some substances can be protracted, onset of the sleep disturbance can occur 4 weeks after cessation of substance use, and the disturbance may have features atypical of other sleep disorders (e.g., atypical age at onset or course).

The high impact of sleep disturbance in Substance Use Disorder is reflected by the development of specialized sleep questionnaires, such as for instance the Substance Use Sleep Scale (SUSS). The SUSS questionnaire comprises 23 questions and 2 domains: “Mind and Body Sleep Problems” and “Substance-Related Sleep Problems”. Scoring ranges from 0-23, where lower scores denote better sleep, and higher scores denote worse sleep.

Treating a patient suffering from Substance Use Disorder and associated sleep disturbance with 5-MeO-DMT or a pharmaceutically acceptable salt thereof reduces or eliminates the sleep disturbance and leads to an improvement of the Substance Use Disorder.

The reduction or elimination of sleep disturbance in a patient suffering from Substance Use Disorder is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Substance Use Disorder occurs not later than about 24 hours after the last administration of 5-MeO-DMT or a pharmaceutically acceptable salt thereof. The reduction or elimination of sleep disturbance in a patient suffering from Substance Use Disorder preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Substance Use Disorder is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Substance Use Disorder, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Substance Use Disorder, as reflected by a reduction in the CGI-S score, occurs not later than about 24 hours after the last administration of 5-MeO-DMT or a pharmaceutically acceptable salt thereof. An improvement in sleep disturbance in a patient suffering from Substance Use Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Substance Use Disorder, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Substance Use Disorder is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Substance Use Disorder as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Substance Use Disorder as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Substance Use Disorder as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is an important disease aspect in patients with Substance Use Disorder. An improvement in sleep disturbance will therefore also lead to an improvement of the Substance Use Disorder. Since sleep disturbance furthermore also affects other aspects of Substance Use Disorder, the inventors conclude that the improvement in sleep disturbance, in particular the reduction or elimination of reduced sleep, will additionally contribute to an overall improvement of the Substance Use Disorder.

An improvement of the Substance Use Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Substance Use Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Substance Use Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Substance Use Disorder in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Psychotic Disorders

Psychotic Disorders are severe mental disorders that cause abnormal thinking and perceptions. Psychotic disorders are characterised by significant impairments in reality testing and alterations in behaviour manifest in positive symptoms such as persistent delusions, persistent hallucinations, disorganised thinking (typically manifest as disorganised speech), grossly disorganised behaviour, and experiences of passivity and control, negative symptoms such as blunted or flat affect and avolition, and psychomotor disturbances.

The patient suffering from a Psychotic Disorder may suffer from a treatment resistant form of the disorder.

Insomnia and nightmare disorder are the most common sleep disturbances associated with psychotic disorders, but also sleep-related hallucinations, excessive sleepiness disorders, restless leg syndrome, periodic limb movement disorder, bruxism, sleep paralysis, night terror or circadian rhythm disorders can be found as comorbidity. Often, patients not only suffer from one isolated sleep disturbance but from a multiplicity of disturbances. The majority of sleep disturbances are rated as severe in their chronicity, frequency, and distress or impairment.

Due to the enormous impact sleep disturbances might have on the course of Psychotic Disorders, sleep quality of patients suffering from Psychotic Disorders is assessed by rating scales commonly used for the diagnosis or the assessment of sleep disorders, such as, for example the PSQI or the Sleep 50.

Brain imaging of patients suffering from psychosis by functional magnetic resonance imaging of brain resting state networks, reveals profound alterations in distinct regions within and/or between the central executive network, the default mode network and the salience network. Even in patient populations at risk for psychosis, alterations in resting state networks can be identified.

Treating a patient suffering from a Psychotic Disorder, including a treatment resistant form of the disorder, and associated sleep disturbance with 5-MeO-DMT or a pharmaceutically acceptable salt thereof reduces or eliminates the sleep disturbance and leads to an improvement of the Psychotic Disorder.

The reduction or elimination of sleep disturbance in a patient suffering from a Psychotic Disorder is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from a Psychotic Disorder occurs not later than about 24 hours after the last administration of 5-MeO-DMT or a pharmaceutically acceptable salt thereof. The reduction or elimination of sleep disturbance in a patient suffering from a Psychotic Disorder preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from a Psychotic Disorder is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Psychotic Disorder, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Psychotic Disorder, as reflected by a reduction in the CGI-S score, occurs not later than about 24 hours after the last administration of 5-MeO-DMT or a pharmaceutically acceptable salt thereof. An improvement in sleep disturbance in a patient suffering from Psychotic Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from a Psychotic Disorder, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from a Psychotic Disorder is reflected by an improvement in the Pittsburgh Sleep Quality Index (PSQI) global score on day 1, for instance, after about 24 hours; on day 7; on day 14 and/or on day 28 after the last administration of 5-MeO-DMT or a pharmaceutically acceptable salt thereof, wherein the recall period spans from the time point when acute psychedelic experiences have subsided after the last administration to the assessment time point.

A reduction or elimination of sleep disturbance in a patient suffering from a Psychotic Disorder as reflected by an improvement in the PSQI global score occurs not later than about 24 hours after the last administration of 5-MeO-DMT or a pharmaceutically acceptable salt thereof, wherein the recall period spans from the time point when acute psychedelic experiences have subsided after the last administration to the assessment time point. The reduction or elimination of sleep disturbance in a patient suffering from a Psychotic Disorder as reflected by an improvement in the score of the PSQI preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from a Psychotic Disorder as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, the occurrence of various forms of sleep disturbance is an important disease aspect in patients with a Psychotic Disorder. An improvement in sleep disturbance will therefore also lead to an improvement of the Psychotic Disorder. Since sleep disturbance furthermore also affects other aspects of a Psychotic Disorder, the inventors conclude that the improvement in sleep disturbance will additionally contribute to an overall improvement of the Psychotic Disorder.

An improvement of the Psychotic Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Psychotic Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Psychotic Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of a Psychotic Disorder in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Schizophrenia is a severe mental health condition characterised by disturbances in multiple mental modalities, including thinking, perception, self-experience, cognition, volition, affect and behaviour. Psychomotor disturbances, including catatonia, may be present.

The patient suffering from Schizophrenia may suffer from a treatment resistant form of the disorder.

Abnormal resting state functional connectivity, particularly within and/or between the default mode network, the frontoparietal network and the salience network, is reported in individuals with schizophrenia. In individuals at high risk for psychosis and in those diagnosed with schizophrenia, several studies have found associations between sleep disturbances and symptom severity.

Often, psychotic experiences interfere with the ability to sleep well, and resulting sleep problems lead to daytime tiredness, thus impairing the ability of schizophrenia patients to cope with their psychotic symptoms.

Insomnia is the most strongly associated sleep disturbance with schizophrenia.

Patients with schizophrenia and concurrent sleep disturbances tend to have poorer quality of life, as assessed by physical health, psychological well-being, social relationships, and environmental factors. Schizophrenia patients with sleep disturbances also report significantly lower satisfaction with life, and the presence of insomnia and comorbid nightmares in these patients has been associated with an increased risk for suicide attempts.

Sleep disturbance is an integral aspect in patients suffering from Schizophrenia any may be assessed by common sleep scales for instance by the Pittsburgh Sleep Quality Index (PSQI).

Treating a patient suffering from Schizophrenia, including a treatment resistant form of the disorder, and associated sleep disturbance with 5-MeO-DMT or a pharmaceutically acceptable salt thereof reduces or eliminates the sleep disturbance and leads to an improvement of the Schizophrenia.

The reduction or elimination of sleep disturbance in a patient suffering from Schizophrenia is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Schizophrenia occurs not later than about 24 hours after the last administration of 5-MeO-DMT or a pharmaceutically acceptable salt thereof. The reduction or elimination of sleep disturbance in a patient suffering from Schizophrenia preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Schizophrenia is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Schizophrenia, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Schizophrenia, as reflected by a reduction in the CGI-S score, occurs not later than about 24 hours after the last administration of 5-MeO-DMT or a pharmaceutically acceptable salt thereof. An improvement in sleep disturbance in a patient suffering from Schizophrenia, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Schizophrenia, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Schizophrenia is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Schizophrenia as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Schizophrenia as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Schizophrenia as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is central to the clinical picture of Schizophrenia. An improvement in sleep disturbance will therefore also lead to an improvement of the Schizophrenia. Since sleep disturbance furthermore also affects other aspects of Schizophrenia, the inventors conclude that the improvement in sleep disturbance will additionally contribute to an overall improvement of the Schizophrenia.

An improvement of the Schizophrenia in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Schizophrenia in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Schizophrenia in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Schizophrenia in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Huntington's Disease

Huntington's Disease (HD) is a neurodegenerative autosomal dominant disorder, characterized by involuntary choreatic movements. HD is associated with cognitive and behavioural disturbances. In fact, progressive cognitive impairment is a core feature of HD, with early changes in executive function (i.e., processing speed, organization, and planning). Cognitive and associated behavioural changes often precede the emergence of the typical motor abnormalities.

Other common symptoms of HD are circadian and sleep abnormalities, which are observed from the earliest stages of the disease or even before the occurrence of clinical symptoms. Up to 90% of patients report sleep problems that are evaluated as important by over half of them. Studies have demonstrated that some sleep disorders are found in the very early phase of HD and even in carriers of the mutation at the premorbid stage.

The most common sleep problems reported by HD patients include insomnia, difficulties in falling asleep, frequent nocturnal awakenings, and excessive daytime sleepiness. Also, specific changes in sleep architecture have been identified in HD.

Sleep disturbance in Huntington's Disease can be assessed using a detailed self-report questionnaire for assessment of sleep problems specific for HD patients (Goodman et al. Identifying sleep disturbances in Huntington's disease using a simple disease-focused questionnaire. PLoS Curr. 2010 Oct. 15; 2: RRN1189). This questionnaire contains 45 questions grouped into four subcategories: quality of sleep (e.g., difficulty falling asleep or maintaining sleep), motor activity (e.g., painful muscle cramps in the arms or legs causing waking at night), abnormal nocturnal behaviour (e.g., acting out dreams, injury to self or others while dreaming), and other aspects of disturbed sleep (e.g., nocturia, numbness, paraesthesia, sleep apnoea, and daytime somnolence).

Furthermore, assessment of sleep disturbance can be by the Pittsburgh Sleep Quality Index (PSQI).

Resting-state functional connectivity architecture is profoundly altered in HD. Resting state networks subserving sensory-motor abilities and cognitive functions are involved in the pathophysiological mechanisms of HD. HD shows an early and widespread disruption of large-scale cognitive networks, related to cognitive and disease burden scores. The default mode network is affected. For instance, in manifest HD, DMN-key brain regions (i.e., ventromedial prefrontal cortex and angular gyrus) show altered functional connectivity.

Treating a patient suffering from HD and associated sleep disturbance with 5-MeO-DMT or a pharmaceutically acceptable salt thereof reduces or eliminates the sleep disturbance and leads to an improvement of the HD.

The reduction or elimination of sleep disturbance in a patient suffering from HD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from HD occurs not later than about 24 hours after the last administration of 5-MeO-DMT or a pharmaceutically acceptable salt thereof. The reduction or elimination of sleep disturbance in a patient suffering from HD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from HD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from HD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from HD, as reflected by a reduction in the CGI-S score, occurs not later than about 24 hours after the last administration of 5-MeO-DMT or a pharmaceutically acceptable salt thereof. An improvement in sleep disturbance in a patient suffering from HD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from HD, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from HD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from HD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from HD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from HD as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is one of the most common primary complaints by patients with HD. An improvement in sleep disturbance will therefore also lead to an improvement of the HD. Since sleep disturbance furthermore also affects other aspects of HD, the inventors conclude that the improvement in sleep disturbance, in particular the reduction or elimination of reduced sleep, will additionally contribute to an overall improvement of the HD.

An improvement of the HD in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the HD in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the HD in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of HD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Parkinson's Disease (PD)

Parkinson's Disease (PD) is an illness characterized by gradually progressive problems with movement, most commonly involving slowing of movements, a tremor present at rest, and walking instability which can cause falls.

Sleep disturbances occur frequently in PD, causing equivalent or even greater discomfort than motor symptoms and thus are effectively decreasing quality of life in patients and caregivers.

Most common sleep disturbances in PD are insomnia, sleep disordered breathing, excessive daytime sleepiness, REM sleep behavior disorder, and sleep-related movement disorders such as restless legs syndrome.

Sleep in patients with Parkinson's disease can be assessed by rating scales especially developed for this patient population, such as for example the Unified Parkinson's Disease Rating Scale-Movement Disorder Society (UPDRS-MDS), the Parkinson's Disease Sleep Scale (PDSS-2) and also by the Pittsburgh Sleep Quality Index (PSQI).

The Unified Parkinson's Disease Rating Scale-Movement Disorder Society (UPDRS-MDS) comprises four parts considering non-motor experiences of daily living, motor experiences of daily living, motor examination and motor complications. Sleep problems, daytime sleepiness and fatigue are assessed as part of the non-motor experiences item. Each sign or symptom is rated on a 5-point scale (ranging from “Normal” (“0”), “Slight” (“1”), “Mild” (2), “Moderate” (“3”) to “Severe” (“4”)).

The Parkinson's Disease Sleep Scale (PDSS-2) was developed to quantify disease-specific nocturnal sleep problems during the past week, allowing the patient to rate disturbances from “Very often”, “Often”, “Sometimes”, “Occasionally”, to “Never”.

Neuroimaging demonstrates that sleep disturbances lead to an alteration in functional connectivity of resting state networks. In PD patients, a functional reorganization of resting state networks, critical to generate and maintain an efficient behavioral and cognitive performance, is observed.

Analysis of resting state networks using functional magnetic resonance imaging show that PD-related cognitive deficits are associated with patterns of altered connectivity within and/or between resting-state functional connectivity networks, such as the default mode network, dorsal attention networks or frontoparietal network.

Levodopa, which improves the clinical status of Parkinson patients, has the potential to influence functional connectivity of resting state networks in Parkinson patients.

Therefore, influencing those networks by a therapy according to the invention in PD patients will lead to an improvement of the cognitive dysfunction, and also to an improvement of the symptoms of PD.

Treating a patient suffering from PD and associated sleep disturbance with 5-MeO-DMT or a pharmaceutically acceptable salt thereof reduces or eliminates the sleep disturbance and leads to an improvement of the PD.

The reduction or elimination of sleep disturbance in a patient suffering from PD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from PD occurs not later than about 24 hours after the last administration of 5-MeO-DMT or a pharmaceutically acceptable salt thereof. The reduction or elimination of sleep disturbance in a patient suffering from PD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from PD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from PD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from PD, as reflected by a reduction in the CGI-S score, occurs not later than about 24 hours after the last administration of 5-MeO-DMT or a pharmaceutically acceptable salt thereof. An improvement in sleep disturbance in a patient suffering from PD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from PD, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from PD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from PD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from PD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from PD as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is one of the most common primary complaints by patients with PD. An improvement in sleep disturbance will therefore also lead to an improvement of the PD. Since sleep disturbance furthermore also affects other aspects of PD, the inventors conclude that the improvement in sleep disturbance, in particular the reduction or elimination of reduced sleep, will additionally contribute to an overall improvement of the PD.

An improvement of the PD in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the PD in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the PD in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of PD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Dementia

Dementia is generally characterized by a loss of memory, language, problem-solving and other thinking abilities that are severe enough to interfere with daily life.

Dementia is caused by brain changes which trigger a decline in cognitive abilities and also affect behaviour, feelings and relationships.

Sleep disturbance is common in individuals with dementia, and sleep loss may contribute to or exacerbate the severity of other dementia symptoms. Disturbed sleep is one of the most distressing dementia symptoms. It has a profound negative impact on the quality of life of both patients and their families.

Moreover, it has become increasingly apparent in recent years that sleep is crucial for physiological memory function, especially for memory consolidation.

Sleep disturbances in patients suffering from Dementia are often assessed by proxy/caregiver-rated questionnaires since dementia can impact the patients' ability to accurately recall their sleep.

Dementia can be assessed by, for instance, the Neuropsychiatric Inventory (NPI), administrated to caregivers of dementia patients. The NPI examines 12 sub-domains of behavioural functioning: delusions, hallucinations, agitation/aggression, dysphoria, anxiety, euphoria, apathy, disinhibition, irritability/lability, aberrant motor activity, night-time behavioural disturbances and appetite and eating abnormalities. This wide variety of domains allows for screening of multiple types of dementia.

The Sleep Disorder Inventory (SDI) focusses on sleep disturbances in Dementia patients and consists of 8 items relating to different sleep aspects: “Difficulty falling asleep”, “Getting up during the night”, “Wandering, pacing or getting involved in inappropriate activities at night”, “Awakening you during the night”, “Awakening at night, dressing, and planning to go out, thinking that it is morning and time to start the day”, “Awakening too early in the morning”, “Sleeping excessively during the day”, and “Other night-time behaviors that bother you”. Frequency, severity, and caregiver distress are assessed. The caregiver rates frequency and severity of each of the sleep disturbance symptoms for the last 2 weeks or another appropriate recall window.

Sleep disturbance in Dementia patients can also be assessed by classical sleep disorder questionnaires such as the Pittsburgh Sleep Quality Index (PSQI), assessing sleep quality and disturbances over a 1-month period, the Epworth Sleepiness Scale (ESS), assessing subjective sleepiness over the prior 2 weeks, the Mayo Sleep Questionnaire (MSQ), a 16-item caregiver-rated measure that poses questions about REM sleep behaviour disorder, periodic limb movements during sleep, restless leg syndrome, sleepwalking, obstructive sleep apnoea and sleep related leg cramps in dementia populations, or the Berlin Questionnaire for sleep-related breathing disorders.

Objective measures of sleep include wrist actigraphs and polysomnography (PSG). Actigraphy provides nonintrusive technology to assess sleep-wake cycles in persons with dementia. They are worn for several consecutive days, like a watch.

Dementia affects various functional and structural connectivity networks in the brain, as can be shown by magnetic resonance imaging studies. Alterations within and/or between the default mode network (DMN), the salience network, and the central executive network (CEN) are observed.

Treating a patient suffering from Dementia and associated sleep disturbance with 5-MeO-DMT or a pharmaceutically acceptable salt thereof reduces or eliminates the sleep disturbance and leads to an improvement of the Dementia.

The reduction or elimination of sleep disturbance in a patient suffering from Dementia is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Dementia occurs not later than about 24 hours after the last administration of 5-MeO-DMT or a pharmaceutically acceptable salt thereof. The reduction or elimination of sleep disturbance in a patient suffering from Dementia preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Dementia is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Dementia, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Dementia, as reflected by a reduction in the CGI-S score, occurs not later than about 24 hours after the last administration of 5-MeO-DMT or a pharmaceutically acceptable salt thereof. An improvement in sleep disturbance in a patient suffering from Dementia, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Dementia, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Dementia is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Dementia as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Dementia as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Dementia as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance and Dementia are closely related. An improvement in sleep disturbance will therefore also lead to an improvement of the Dementia. Since sleep disturbance furthermore also affects other aspects of the Dementia, such as cognitive function, the inventors conclude that the improvement in sleep disturbance, in particular the reduction or elimination of reduced sleep, will additionally contribute to an overall improvement of the Dementia.

An improvement of the Dementia in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Dementia in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Dementia in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Dementia in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Alzheimer's Dementia (AD) is a highly prevalent neurodegenerative disorder with an insidious onset and gradual progression of cognitive deficits. Sleep disturbance is typically associated with AD. The most frequent reports are of nocturnal awakening, difficulties falling asleep and frequent naps during the day. AD is in particular associated with Irregular Sleep Wake Rhythm Disorder.

Sleep disturbances appear early in AD and progressively worsen with the progression of the disease. The worsening of sleep disorders in the context of late-stage AD is strongly predictive of mortality.

Sleep disturbances in patients suffering from AD are often assessed by proxy/caregiver-rated questionnaires since dementia can impact the patients' ability to accurately recall their sleep.

AD can be assessed by, for instance, the Neuropsychiatric Inventory (NPI).

Sleep disturbance in AD patients can be assessed by the Sleep Disorder Inventory (SDI) and also by classical sleep disorder questionnaires such as the Pittsburgh Sleep Quality Index (PSQI), the Epworth Sleepiness Scale (ESS), the Mayo Sleep Questionnaire (MSQ), or the Berlin Questionnaire for sleep-related breathing disorders.

Objective measures of sleep include wrist actigraphs and polysomnography (PSG).

The relationship between sleep disturbances and AD is likely bidirectional. Beta-amyloid (A•), a key pathological protein in AD pathology, was found to be significantly higher in participants who underwent 1 night of sleep deprivation. The study also found a correlation between length of sleep and clearance of A•. It is hypothesised that sleep deprivation may therefore also contribute to AD development.

AD affects various functional and structural connectivity networks in the brain which are associated with the topography, clinical phenotype, and severity of the disease. Magnetic resonance imaging studies have demonstrated successive structural and functional disconnection among brain regions supporting the idea that AD is a disconnection syndrome. The core brain network targeted by AD pathology plays a key role in sleep-wake cycling: sleep disturbance in AD may therefore establish a pathophysiological ‘vicious cycle’, whereby loss of normal restorative functions of sleep amplifies the neurodegenerative process within vulnerable neural circuitry. Moreover, alterations within and/or between the default mode network (DMN), salience network, and central executive network (CEN) are observed both in patients with AD and individuals who were at high risk for developing AD.

Therefore, influencing those networks by a therapy according to the invention will lead to an improvement of the sleep disturbance, and also to an improvement of the symptoms of AD.

Treating a patient suffering from AD and associated sleep disturbance with 5-MeO-DMT or a pharmaceutically acceptable salt thereof reduces or eliminates the sleep disturbance and leads to an improvement of the AD.

The reduction or elimination of sleep disturbance in a patient suffering from AD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from AD occurs not later than about 24 hours after the last administration of 5-MeO-DMT or a pharmaceutically acceptable salt thereof. The reduction or elimination of sleep disturbance in a patient suffering from AD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from AD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from AD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from AD, as reflected by a reduction in the CGI-S score, occurs not later than about 24 hours after the last administration of 5-MeO-DMT or a pharmaceutically acceptable salt thereof. An improvement in sleep disturbance in a patient suffering from AD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from AD, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from AD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from AD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from AD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from AD as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance and AD are closely related. An improvement in sleep disturbance will therefore also lead to an improvement of the AD. Since sleep disturbance furthermore also affects other aspects of the AD, such as cognitive function, the inventors conclude that the improvement in sleep disturbance, in particular the reduction or elimination of reduced sleep, will additionally contribute to an overall improvement of the AD.

An improvement of the Alzheimer's Dementia in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Alzheimer's Dementia in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Alzheimer's Dementia in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of AD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Parkinson's Disease Dementia is characterized by changes in thinking and behaviour in a patient with a diagnosis of Parkinson's disease, an illness characterized by gradually progressive problems with movement, most commonly involving slowing of movements, a tremor present at rest, and walking instability which can cause falls.

In the vast majority of patients with PD cognitive function deteriorates over time. By 20 years of disease duration, up to 80% of patients develop Parkinson's Disease Dementia, with a mean time from onset of PD to dementia of 10 years.

The most common sleep complaint in Parkinson's Disease Dementia patients is frequent nocturnal awakenings and sleep fragmentation. PDD patients complain of frequent awakenings which, if prolonged, can result in an overall reduction in sleep time eventually causing excessive daytime sleepiness.

Sleep disturbances in patients suffering from Parkinson's Disease Dementia are often assessed by proxy/caregiver-rated questionnaires since dementia can impact the patients' ability to accurately recall their sleep.

Assessment can, for instance, be by using the Unified Parkinson's Disease Rating Scale-Movement Disorder Society (UPDRS-MDS) comprises four parts considering non-motor experiences of daily living, motor experiences of daily living, motor examination and motor complications. Sleep problems, daytime sleepiness and fatigue are assessed as part of the non-motor experiences item. Each sign or symptom is rated on a 5-point scale (ranging from “Normal” (“0”), “Slight” (“1”), “Mild” (2), “Moderate” (“3”) to “Severe” (“4”)).

Sleep disturbance in Parkinson's Disease Dementia patients can also be assessed by classical sleep disorder questionnaires such as the Pittsburgh Sleep Quality Index (PSQI).

Objective measures of sleep include wrist actigraphs and polysomnography (PSG).

Parkinson's Disease Dementia is associated with reduced connectivity in networks relevant to cognition, most prominently the default mode network, which also affected in patients suffering from sleep disturbances.

Treating a patient suffering from Parkinson's Disease Dementia and associated sleep disturbance with 5-MeO-DMT or a pharmaceutically acceptable salt thereof reduces or eliminates the sleep disturbance and leads to an improvement of the Parkinson's Disease Dementia.

The reduction or elimination of sleep disturbance in a patient suffering from Parkinson's Disease Dementia is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Parkinson's Disease Dementia occurs not later than about 24 hours after the last administration of 5-MeO-DMT or a pharmaceutically acceptable salt thereof. The reduction or elimination of sleep disturbance in a patient suffering from Parkinson's Disease Dementia preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Parkinson's Disease Dementia is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Parkinson's Disease Dementia, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Parkinson's Disease Dementia, as reflected by a reduction in the CGI-S score, occurs not later than about 24 hours after the last administration of 5-MeO-DMT or a pharmaceutically acceptable salt thereof. An improvement in sleep disturbance in a patient suffering from Parkinson's Disease Dementia, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Parkinson's Disease Dementia, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Parkinson's Disease Dementia is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Parkinson's Disease Dementia as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from PDD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Parkinson's Disease Dementia as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is an important aspect of Parkinson's Disease Dementia. An improvement in sleep disturbance will therefore also lead to an improvement of the Parkinson's Disease Dementia. Since sleep disturbance furthermore also affects other aspects of Parkinson's Disease Dementia, such as cognitive function, the inventors conclude that the improvement in sleep disturbance, in particular the reduction or elimination of reduced sleep, will additionally contribute to an overall improvement of the Parkinson's Disease Dementia.

An improvement of the Parkinson's Disease Dementia in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Parkinson's Disease Dementia in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Parkinson's Disease Dementia in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Parkinson's Disease Dementia in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Dementia with Lewy Bodies (DLB) is a type of dementia characterized by changes in sleep, behaviour, cognition, movement, and regulation of automatic bodily functions.

Core features include REM sleep behaviour disorder as further discussed below as well as visual hallucinations, marked fluctuations in attention or alertness, and parkinsonism (slowness of movement, trouble walking, or rigidity).

Memory loss is not always an early symptom. The disease worsens over time and is usually diagnosed when cognitive impairment interferes with normal daily functioning.

In patients suffering from DLB, resting state network (RSN) alterations can be identified. Functional MRI studies show changes in functional connectivity within and/or between the default mode network, salience network, executive network and basal ganglia/limbic network.

As indicated, sleep disturbance is a hallmark of DLB.

With a prevalence of 76%, REM sleep behavior disorder (RBD) is an established core clinical feature. It often begins many years before other symptoms and may thus precede cognitive decline.

Typically, RBD is a parasomnia manifested by recurrent dream enactment behavior that includes movements mimicking dream content and associated with an absence of normal REM sleep atonia; the latter can be clinically verified by polysomnography. If the polysomnography shows REM sleep without atonia in a person with dementia and a history of REM sleep behaviour disorder, there is a more than 90% likelihood of a synucleinopathy, sufficient to justify a probable DLB diagnosis even in the absence of any other core feature or biomarker.

Hypersomnia or excessive daytime sleepiness is considered one supportive clinical feature of this disease. It is commonly present, sometimes early in patients with DLB. Although lacking diagnostic specificity, it may indicate DLB in a patient with dementia, particularly when it persists over time or occurs in combination with other clinical features of DLB.

A wide range of other sleep or sleep/wakefulness disturbances is observed in DLB, including insomnia, sleep fragmentation, motor-related sleep disturbances, restless legs syndrome or periodic limb movements, which have profound negative impact on quality of life in both patients and their families. Obstructive sleep apnoea might occur in up to a third of patients. Patients experience excessive daytime somnolence, worsening of cognitive function, unrefreshing sleep, and early morning headaches.

Sleep disturbances in patients suffering from DLB are often assessed by proxy/caregiver-rated questionnaires since dementia can impact the patients' ability to accurately recall their sleep.

DLB can be assessed by, for instance, the Neuropsychiatric Inventory (NPI).

Sleep disturbance in DLB patients can be assessed by the Sleep Disorder Inventory (SDI) and also by classical sleep disorder questionnaires such as the Pittsburgh Sleep Quality Index (PSQI), the Epworth Sleepiness Scale (ESS), the Mayo Sleep Questionnaire (MSQ), or the Berlin Questionnaire for sleep-related breathing disorders.

Objective measures of sleep include wrist actigraphs and polysomnography (PSG).

Treating a patient suffering from DLB and associated sleep disturbance with 5-MeO-DMT or a pharmaceutically acceptable salt thereof reduces or eliminates the sleep disturbance and leads to an improvement of the DLB.

The reduction or elimination of sleep disturbance in a patient suffering from DLB is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from DLB occurs not later than about 24 hours after the last administration of 5-MeO-DMT or a pharmaceutically acceptable salt thereof. The reduction or elimination of sleep disturbance in a patient suffering from DLB preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from DLB is observed on day 7 after the last administration of 5-MeO-DMT or a pharmaceutically acceptable salt thereof and preferably persists until at least 14 days, more preferably until at least 28 days.

An improvement in sleep disturbance in a patient suffering from DLB, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from DLB, as reflected by a reduction in the CGI-S score, occurs not later than about 24 hours after the last administration of 5-MeO-DMT or a pharmaceutically acceptable salt thereof. An improvement in sleep disturbance in a patient suffering from DLB, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from DLB, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from DLB is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from DLB as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from DLB as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from DLB as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance play a central role in DLB. An improvement in sleep disturbance will therefore also lead to an improvement of the DLB. Since sleep disturbance furthermore also affects other aspects of DLB, such as cognitive function, the inventors conclude that the improvement in sleep disturbance, in particular the reduction or elimination of reduced sleep, will additionally contribute to an overall improvement of the DLB.

An improvement of the Dementia with Lewy Bodies in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Dementia with Lewy Bodies in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Dementia with Lewy Bodies in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of DLB in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Vascular Dementia is a common type of dementia characterized by problems with reasoning, planning, judgment, memory and other thought processes. Vascular Dementia is due to brain parenchyma injury resulting from one or more cerebrovascular event (ischemic or haemorrhagic). The vascular aetiology is very varied, ranging from microvascular disease to large vessel stroke.

Frequently observed sleep disturbances in patients suffering from Vascular Dementia are insomnia, excessive daytime sleepiness and obstructive sleep apnoea.

These sleep disturbances, and in particular severe daytime sleepiness, are strong predictors of Vascular Dementia.

Moreover, insomnia has also a direct impact on disease progression. This can be explained by insomnia-related disruption of subcortical circuits, which are related to the sleep cycle. Changes in cerebral blood perfusion and systemic inflammation due to insomnia may promote small vessel disease and ischemic stroke, thereby increasing the risk of Vascular Dementia.

In addition to its effect on the incidence, insomnia also influences the prognosis following dementia diagnosis. Higher mortality and rates of admission to long-term care facilities suggest that worse prognosis can be expected in patients with pre-existing insomnia at the time of dementia diagnosis.

Sleep disturbances in patients suffering from Vascular Dementia are often assessed by proxy/caregiver-rated questionnaires since dementia can impact the patients' ability to accurately recall their sleep.

Sleep disturbance in Vascular Dementia patients can be assessed, for instance, by the Pittsburgh Sleep Quality Index (PSQI).

Objective measures of sleep include wrist actigraphs and polysomnography (PSG).

In Vascular Dementia patients, a direct association of insomnia with white matter lesions and cerebral microbleeds (Vascular Dementia brain imaging biomarkers) can be observed.

Functional magnetic resonance imaging reveals altered functional connectivity of resting state networks after a cerebrovascular insult. Patients show an abnormal functional connectivity within and/or between the DMN and other resting state networks.

Treating a patient suffering from Vascular Dementia and associated sleep disturbance with 5-MeO-DMT or a pharmaceutically acceptable salt thereof reduces or eliminates the sleep disturbance and leads to an improvement of the Vascular Dementia.

The reduction or elimination of sleep disturbance in a patient suffering from Vascular Dementia is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Vascular Dementia occurs not later than about 24 hours after the last administration of 5-MeO-DMT or a pharmaceutically acceptable salt thereof. The reduction or elimination of sleep disturbance in a patient suffering from Vascular Dementia preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Vascular Dementia is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Vascular Dementia, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Vascular Dementia, as reflected by a reduction in the CGI-S score, occurs not later than about 24 hours after the last administration of 5-MeO-DMT or a pharmaceutically acceptable salt thereof. An improvement in sleep disturbance in a patient suffering from Vascular Dementia, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Vascular Dementia, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Vascular Dementia is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Vascular Dementia as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Vascular Dementia as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Vascular Dementia as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, there is a strong connection between sleep disturbance and Vascular Dementia. An improvement in sleep disturbance will therefore also lead to an improvement of the Vascular Dementia. Since sleep disturbance furthermore also affects other aspects of the Vascular Dementia, such as cognitive function, the inventors conclude that the improvement in sleep disturbance, in particular the reduction or elimination of reduced sleep, will additionally contribute to an overall improvement of the Vascular Dementia.

An improvement of the Vascular Dementia in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Vascular Dementia in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Vascular Dementia in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Vascular Dementia in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

A Fronto-Temporal Dementia (FTD) is a neurodegenerative disorder caused by progressive nerve cell loss in the brain's frontal or temporal lobes that constitute a leading cause of younger onset dementia.

Resting state-functional magnetic resonance imaging (Rs-fMRI) analysis of individuals suffering from a FTD reveals global functional deterioration and a loss of network integration. Altered functional connectivity was mainly identified within and/or between the attention network, the default mode network and in particular the salience network.

Sleep disturbance is a substantial clinical issue in FTD.

Sleep disturbances in patients suffering from FTD are often assessed by proxy/caregiver-rated questionnaires since dementia can impact the patients' ability to accurately recall their sleep.

Sleep disturbance in FTD patients can be assessed, for instance, by the Pittsburgh Sleep Quality Index (PSQI).

Objective measures of sleep include wrist actigraphs and polysomnography (PSG).

Difficulty falling or staying sleep at night and excessive daytime somnolence is observed in three-quarters of patients with FTD.

The disease process in FTD targets sleep circuitry in hypothalamus and basal forebrain and patients with FTD have been reported to develop excessive somnolence as well as narcolepsy-like attacks, insomnia and other sleep-related symptoms.

Treating a patient suffering from FTD and associated sleep disturbance with 5-MeO-DMT or a pharmaceutically acceptable salt thereof reduces or eliminates the sleep disturbance and leads to an improvement of the FTD.

The reduction or elimination of sleep disturbance in a patient suffering from FTD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from FTD occurs not later than about 24 hours after the last administration of 5-MeO-DMT or a pharmaceutically acceptable salt thereof. The reduction or elimination of sleep disturbance in a patient suffering from FTD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from FTD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from FTD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from FTD, as reflected by a reduction in the CGI-S score, occurs not later than about 24 hours after the last administration of 5-MeO-DMT or a pharmaceutically acceptable salt thereof. An improvement in sleep disturbance in a patient suffering from FTD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from FTD, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from FTD is reflected by an improvement in the Pittsburgh Sleep Quality Index (PSQI) global, in particular by a decrease to 5 or below, score on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from FTD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from FTD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from FTD as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is substantial clinical issue in FTD. An improvement in sleep disturbance will therefore also lead to an improvement of the FTD. Since sleep disturbance furthermore also affects other aspects of the FTD, such as cognitive function, the inventors conclude that the improvement in sleep disturbance, in particular the reduction or elimination of reduced sleep, will additionally contribute to an overall improvement of the FTD.

An improvement of FTD in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of FTD in a patient also suffering from associated sleep disturbance, as reflected by a reduction in the CGI-S score, occurs not later than about 24 hours after the last administration of 5-MeO-DMT or a pharmaceutically acceptable salt thereof. An improvement of FTD in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of FTD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Eating Disorders

An Eating Disorder is a mental disorder characterised by abnormal eating behaviours that negatively affect a person's physical or mental health.

The patient suffering from an Eating Disorder may suffer from a treatment resistant form of the disorder.

In patients suffering from Eating Disorders, results from functional network connectivity studies indicate disrupted resting-state connectivity within and/or between executive networks, the default-mode network and the salience network.

Sleep disturbance (such as insomnia, changes in REM duration and decreased sleep efficiency) is a common symptom, seen in 57% of people with eating disorders.

It is particularly prevalent in people with anorexia but is also seen in sufferers of bulimia nervosa and binge Eating Disorder.

There is a link between malnutrition in anorexia and sleep disturbances. A study found women suffering from anorexia had higher levels of orexin, which is increased in the body to promote wakefulness and feeding. A correlation was found between orexin concentration and reduced sleep quality. The study also concluded that it predicted poorer treatment outcome.

Eating Disorders are associated with sleep disturbances. This is supported by the use of separate sleep scales, such as, for example, the PSQI for the assessment of sleep disturbances in patients suffering from Eating Disorders.

Treating a patient suffering from an Eating Disorder, including a treatment resistant form of the disorder, and associated sleep disturbance with 5-MeO-DMT or a pharmaceutically acceptable salt thereof reduces or eliminates the sleep disturbance and leads to an improvement of the Eating Disorder.

The reduction or elimination of sleep disturbance in a patient suffering from an Eating Disorder is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from an Eating Disorder occurs not later than about 24 hours after the last administration of 5-MeO-DMT or a pharmaceutically acceptable salt thereof. The reduction or elimination of sleep disturbance in a patient suffering from an Eating Disorder preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from an Eating Disorder is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Eating Disorder, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from an Eating Disorder, as reflected by a reduction in the CGI-S score, occurs not later than about 24 hours after the last administration of 5-MeO-DMT or a pharmaceutically acceptable salt thereof. An improvement in sleep disturbance in a patient suffering from an Eating Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from an Eating Disorder, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from an Eating Disorder is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from an Eating Disorder as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from an Eating Disorder as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from an Eating Disorder as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, there is a link between sleep disturbance and Eating Disorders. An improvement in sleep disturbance will therefore also lead to an improvement of the Eating Disorder. Since sleep disturbance furthermore also affects other aspects of the Eating Disorder, the inventors conclude that the improvement in sleep disturbance, in particular the reduction or elimination of reduced sleep, will additionally contribute to an overall improvement of the Eating Disorder.

An improvement of the Eating Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Eating Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Eating Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of an Eating Disorder in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Attention Deficit Hyperactivity Disorder (ADHD)

Attention Deficit Hyperactivity Disorder (ADHD) is a condition that affects a patient's behaviour. A patient suffering from ADHD can seem restless, may have trouble concentrating and may act on impulse.

Resting state network analysis in patients suffering from ADHD reveals dysfunctional connectivity across multiple brain resting state networks, in particular dysfunctional connectivity within and/or between distinct regions of the default mode network, the dorsal attention network and the salience network.

Patients with ADHD may also have additional problems, such as sleep and anxiety disorders.

Sleep issues are reported in 25-50% of individuals with ADHD. The most common types of sleep disturbances associated with ADHD are sleep-disordered breathing, restless leg syndrome, circadian rhythm sleep disorders, insomnia and narcolepsy.

Sleep problems can also lead to the development of ADHD, with behavioural interventions improving symptoms in some individuals.

Treating a patient suffering from ADHD and associated sleep disturbance with 5-MeO-DMT or a pharmaceutically acceptable salt thereof reduces or eliminates the sleep disturbance and leads to an improvement of the ADHD.

The reduction or elimination of sleep disturbance in a patient suffering from ADHD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from ADHD occurs not later than about 24 hours after the last administration of 5-MeO-DMT or a pharmaceutically acceptable salt thereof. The reduction or elimination of sleep disturbance in a patient suffering from ADHD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from ADHD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from ADHD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from ADHD, as reflected by a reduction in the CGI-S score, occurs not later than about 24 hours after the last administration of 5-MeO-DMT or a pharmaceutically acceptable salt thereof. An improvement in sleep disturbance in a patient suffering from ADHD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from ADHD, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from ADHD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from ADHD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from ADHD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from ADHD as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, there is a connection between sleep disturbance and ADHD. An improvement in sleep disturbance will therefore also lead to an improvement of the ADHD. Since sleep disturbance furthermore also affects other aspects of ADHD, such as cognitive function, the inventors conclude that the improvement in sleep disturbance, in particular the reduction or elimination of reduced sleep, will additionally contribute to an overall improvement of the ADHD.

An improvement of the ADHD in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the ADHD in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Attention Deficit Hyperactivity Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of ADHD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Personality Disorders

Schizotypal Personality Disorder is a mental health condition marked by a consistent pattern of intense discomfort with relationships and social interactions. Patients suffering from Schizotypal Personality Disorder have unusual thoughts, speech, and behaviours, which hinder their ability to form and maintain relationships.

The patient suffering from Schizotypal Personality Disorder may suffer from a treatment resistant form of the disorder.

Schizotypal Personality Disorder features are significantly associated with insomnia severity.

In patients with Schizotypal Personality Disorder, DMN functional connectivity, particularly that involving cognitive or emotional regulation, is altered. Neuroimaging analysis by fMRI further reveals alterations within and/or between frontoparietal network and dorsal attention network.

Treating a patient suffering from Schizotypal Personality Disorder, including a treatment resistant form of the disorder, and associated sleep disturbance with 5-MeO-DMT or a pharmaceutically acceptable salt thereof reduces or eliminates the sleep disturbance and leads to an improvement of the Schizotypal Personality Disorder.

The reduction or elimination of sleep disturbance in a patient suffering from Schizotypal Personality Disorder is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Schizotypal Personality Disorder occurs not later than about 24 hours after the last administration of 5-MeO-DMT or a pharmaceutically acceptable salt thereof. The reduction or elimination of sleep disturbance in a patient suffering from Schizotypal Personality Disorder preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Schizotypal Personality Disorder is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Schizotypal Personality Disorder, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Schizotypal Personality Disorder, as reflected by a reduction in the CGI-S score, occurs not later than about 24 hours after the last administration of 5-MeO-DMT or a pharmaceutically acceptable salt thereof. An improvement in sleep disturbance in a patient suffering from Schizotypal Personality Disorder, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Schizotypal Personality Disorder, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Schizotypal Personality Disorder is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Schizotypal Personality Disorder as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Schizotypal Personality Disorder as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Schizotypal Personality Disorder as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is an important disease aspect in patients with Schizotypal Personality Disorder. An improvement in sleep disturbance will therefore also lead to an improvement of the Schizotypal Personality Disorder. Since sleep disturbance furthermore also affects other aspects of Schizotypal Personality Disorder, the inventors conclude that the improvement in sleep disturbance, in particular the reduction or elimination of reduced sleep, will additionally contribute to an overall improvement of the Schizotypal Personality Disorder.

An improvement of the Schizotypal Personality Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Schizotypal Personality Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Schizotypal Personality Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Schizotypal Personality Disorder in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Persistent mood instability, impulsivity, identity disturbance and interpersonal dysfunction are characteristic traits for Borderline Personality Disorder (BPD).

The patient suffering from BPD may suffer from a treatment resistant form of the disorder.

BPD is associated with different types of sleep disturbances, such as disturbances of sleep continuity, altered REM sleep regulation and nightmares.

Sleep problems are clinically pertinent to BPD, as they are linked to functional impairment and emotion dysregulation. Due to the sleep disturbances, patients diagnosed with BPD also experience increased daytime sleepiness, fatigue, and impairment in the completion of daytime activities.

Insomnia can exacerbate symptoms associated with Borderline Personality Disorder.

Sleep disturbances in patients suffering from BPD are assessed by the Borderline Symptom List 95 (BSL-95), a 95 item self-assessment scale for recording the extent of intrapsychic stress in borderline patients.

Patients suffering from sleep disturbances show resting state network alterations as can be demonstrated by fMRI. Also, patients with Borderline Personality Disorder show abnormal connectivity patterns in resting-state networks. Analysis of functional connectivity of brain resting state networks using functional magnetic resonance imaging reveals alterations within and/or between various networks, such as for example the default mode network and salience network.

Treating a patient suffering from BPD, including a treatment resistant form of the disorder, and associated sleep disturbance with 5-MeO-DMT or a pharmaceutically acceptable salt thereof reduces or eliminates the sleep disturbance and leads to an improvement of the BPD.

The reduction or elimination of sleep disturbance in a patient suffering from BPD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from BPD occurs not later than about 24 hours after the last administration of 5-MeO-DMT or a pharmaceutically acceptable salt thereof. The reduction or elimination of sleep disturbance in a patient suffering from BPD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from BPD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from BPD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

In an embodiment, an improvement in sleep disturbance in a patient suffering from BPD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from BPD, as reflected by a reduction in the CGI-S score, occurs not later than about 24 hours after the last administration of 5-MeO-DMT or a pharmaceutically acceptable salt thereof. An improvement in sleep disturbance in a patient suffering from BPD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from BPD, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from BPD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from BPD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from BPD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from BPD as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, there is a connection between sleep disturbance and BPD. An improvement in sleep disturbance will therefore also lead to an improvement of the BPD. Since sleep disturbance furthermore also affects other aspects of BPD, the inventors conclude that the improvement in sleep disturbance, in particular the reduction or elimination of reduced sleep, will additionally contribute to an overall improvement of the BPD.

An improvement of the Borderline Personality Disorder in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Borderline Personality Disorder in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Borderline Personality Disorder in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of BPD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Autism Spectrum Disorder (ASD)

Patients suffering from an Autism Spectrum Disorder (ASD), including autism, Asperger's syndrome and atypical autism, have persistent deficits in the ability to initiate and to sustain reciprocal social interaction and social communication. The condition is moreover characterized by a range of restricted, repetitive, and inflexible patterns of behaviour, interests or activities that are clearly atypical or excessive for the individual's age and sociocultural context.

The onset of the disorder is during the developmental period, but symptoms may not become fully manifest until later. Deficits are sufficiently severe to affect a patient's ability to function in personal, family, social, educational, occupational or other areas.

Patients suffering from ASD may also experience sleep disturbance, in particular insomnia. It takes them longer to fall asleep, and many wake up frequently during the night.

Sleep in patients with ASD may also be less restorative than in the general population. ASD patients spent less time in the rapid eye movement (REM) stage.

Some patients with the condition have sleep apnoea, which causes them to stop breathing several times during the night.

Sleep disturbance in ASD populations can be evaluated by classical sleep disorder questionnaires using the insomnia severity index, or the Pittsburgh Sleep Quality Index.

Sleep disturbance and ASD share a bidirectional relationship. Sleep disturbances can exacerbate autism features.

ASD as a disorder of brain network connectivity. Neuroimaging studies indicate that ASD is related to the anomalous responses in certain brain areas, significant alteration of brain networks, including altered functional connectivity within and/or between the default mode network (DMN) and the sensory processing network, and disturbed neural synchronization between brain areas.

Treating a patient suffering from ASD and associated sleep disturbance with 5-MeO-DMT or a pharmaceutically acceptable salt thereof reduces or eliminates the sleep disturbance and leads to an improvement of the ASD.

The reduction or elimination of sleep disturbance in a patient suffering from ASD is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from ASD occurs not later than about 24 hours after the last administration of 5-MeO-DMT or a pharmaceutically acceptable salt thereof. The reduction or elimination of sleep disturbance in a patient suffering from ASD preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from ASD is observed on day 7 after the last administration of 5-MeO-DMT or a pharmaceutically acceptable salt thereof and preferably persists until at least 14 days, more preferably until at least 28 days.

An improvement in sleep disturbance in a patient suffering from ASD, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from ASD, as reflected by a reduction in the CGI-S score, occurs not later than about 24 hours after the last administration of 5-MeO-DMT or a pharmaceutically acceptable salt thereof. An improvement in sleep disturbance in a patient suffering from ASD, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from ASD, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from ASD is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from ASD as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from ASD as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from ASD as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is one of the most common primary complaints by patients with ASD. An improvement in sleep disturbance will therefore also lead to an improvement of the ASD. Since sleep disturbance furthermore also affects other aspects of ASD, the inventors conclude that the improvement in sleep disturbance, in particular the reduction or elimination of reduced sleep, will additionally contribute to an overall improvement of the ASD.

An improvement of the ASD in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the ASD in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the ASD in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of ASD in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Chronic Fatigue Syndrome

Fatigue describes a feeling of exhaustion, lethargy, and decreased energy. Fatigue is experienced as a weakening or depletion of one's physical or mental resources. Even though considered as normal following a period of exertion, mental or physical, fatigue may occur in the absence of such exertion as a symptom of health conditions.

Chronic fatigue exacerbated by activity is moreover a prominent symptom in Chronic Fatigue Syndrome. In this disorder, severe fatigue is accompanied by neurocognitive, autonomic, and immunological symptoms.

A variety of abnormalities in normal sleep patterns, which may act as perpetuating factors, have been reported in Chronic Fatigue Syndrome patients.

In the early stages of illness, Chronic Fatigue Syndrome patients tend to complain of hypersomnia. This is often followed by a general decrease in sleep efficiency once the illness enters a more chronic stage. Even if patients had many hours of sleep, they tend not to feel refreshed when they wake up.

Compensatory behaviour for the non-restorative night-sleep can lead to alterations in circadian rhythm. Also, vivid dreams, periodic limb movements during sleep and restless legs syndrome are frequently reported.

Fatigue in Patients suffering from Chronic Fatigue Syndrome is commonly assessed by for example the Chalder Fatigue Scale, a 14-item instrument designed to measure the severity of physical and mental symptoms in both clinical and research settings. Respondents are asked to answer questions pertaining to fatigue with one of four response choices: “better than usual,” “no more than usual,” “worse than usual,” or “much worse than usual.”

In terms of scoring, the scale can accommodate two different methods. The first weights the individual's responses as Likert-type items and uses those scores to interpret results. The second ignores the severity of responses and uses a bimodal system to categorize each answer as either problematic or not.

The FACES (Fatigue, Anergy, Consciousness, Energy, Sleepiness) rating scale is a 50 items checklist to distinguish between tiredness, sleepiness, and fatigue. Respondents use a scale ranging from 0 (“not at all”) to 3 (“strongly”) to indicate the degree to which they have experienced each feeling or energy state over the course of the previous week or another appropriate recall period. Higher scores indicate more acute states of tiredness or fatigue, except for those items belonging to the energy subscale (A. Shahid et al., loc. cit.).

Sleep disturbances in Chronic Fatigue Syndrome can be assessed, for instance, using polysomnography, the Epworth Sleepiness scale and the Pittsburgh Sleep Quality Index. Treatment response can be evaluated by above-mentioned quantitative measurements such as polysomnography or actigraphy and/or the use of the above-mentioned questionnaires.

Patients suffering from Chronic Fatigue Syndrome show abnormal resting state functional connectivity, significantly correlated with the severity of their chronic fatigue. Networks involved are also involved in sleep regulation.

Treating a patient suffering from Chronic Fatigue Syndrome and associated sleep disturbance with 5-MeO-DMT or a pharmaceutically acceptable salt thereof reduces or eliminates the sleep disturbance and leads to an improvement of the Chronic Fatigue Syndrome.

The reduction or elimination of sleep disturbance in a patient suffering from Chronic Fatigue Syndrome is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Chronic Fatigue Syndrome occurs not later than about 24 hours after the last administration of 5-MeO-DMT or a pharmaceutically acceptable salt thereof. The reduction or elimination of sleep disturbance in a patient suffering from Chronic Fatigue Syndrome preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Chronic Fatigue Syndrome is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Chronic Fatigue Syndrome, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Chronic Fatigue Syndrome, as reflected by a reduction in the CGI-S score, occurs not later than about 24 hours after the last administration of 5-MeO-DMT or a pharmaceutically acceptable salt thereof. An improvement in sleep disturbance in a patient suffering from Chronic Fatigue Syndrome, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Chronic Fatigue Syndrome, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Chronic Fatigue Syndrome is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Chronic Fatigue Syndrome as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Chronic Fatigue Syndrome as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Chronic Fatigue Syndrome as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is one of the most common primary complaints by patients with Chronic Fatigue Syndrome. An improvement in sleep disturbance will therefore also lead to an improvement of the Chronic Fatigue Syndrome. Since sleep disturbance furthermore also affects other aspects of Chronic Fatigue Syndrome, the inventors conclude that the improvement in sleep disturbance, in particular the reduction or elimination of reduced sleep, will additionally contribute to an overall improvement of the Chronic Fatigue Syndrome.

An improvement of the Chronic Fatigue Syndrome in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Chronic Fatigue Syndrome in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Chronic Fatigue Syndrome in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Chronic Fatigue Syndrome in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### Medical Health Conditions Leading to an Associated Mental or Nervous System Condition

Sleep disturbance may occur in a patient suffering from medical health condition leading to an associated mental or nervous system condition.

Sleep Disturbance in Patients with Traumatic Brain Injury

A traumatic brain injury (TBI) is an injury to the brain caused by an external force.

Traumatic brain injuries are characterized as primary or secondary brain injuries. A primary brain injury refers to the structural damage created during the time of impact from contact, acceleration-deceleration, and/or rotational forces. A secondary brain injury refers to the damage sustained from the subsequent cellular processes that occur from the primary injury (ie, hypoxia and/or raised intracranial pressure).

Sleep disturbances are frequently identified following traumatic brain injury, affecting 30%-70% of persons, and often occur after mild head injury. Insomnia, fatigue, and sleepiness are the most frequent sleep complaints after traumatic brain injury, but also sleep apnea, narcolepsy, periodic limb movement disorder, and parasomnias are reported.

Often, these sleep disturbances develop with time postinjury. For instance, a study revealed that three out of four patients who were initially hospitalized for traumatic brain injury developed sleep-wake disturbances by 6 months postinjury.

The location of the injury within sleep-regulating brain regions can lead to a specific sleep symptom. For instance, hypersomnia can result when areas involving the maintenance of wakefulness are injured. Damage to the basal forebrain, which is involved in sleep initiation, can lead to symptoms of insomnia. Traumatic brain injury may compromise the suprachiasmatic nucleus sand/or its output. This may cause disturbances in the circadian rhythm and, subsequently, a combination of hypersomnia and insomnia.

Sleep disorders in TBI can be evaluated using the Sleep and Concussion Questionnaire, a 12-item measure to identify and quantify changes in sleep in response to concussion and brain injury and, to monitor these changes over time. In initial assessment, • ndings are compared with pre-injury sleep, and for subsequent visits, • ndings are compared with both pre-injury sleep as well as sleep over the time since the most recent visit. Each item is scored from 0 to 3, and scores for all items are summed for a total score out of 26. Total scores of 0-6 indicate no clinically relevant change, scores of 7-14 indicate subclinical change and a recommendation to monitor, scores of 15-20 indicate clinical changes of moderate severity and a recommendation for further assessment, and scores of 21-26 indicate severe clinical changes.

To assess sleep disturbance in patients suffering from sleep disturbance due to TBI, the Pittsburgh Sleep Quality Index (PSQI) is widely used.

Resting state functional connectivity analysis reveals alterations in overall functional connectivity within and/or between resting state networks, such as the DMN, the frontoparietal network, the executive network, and the sensory motor network. For instance, highly connected regions that can be found within the DMN are particularly susceptible to alterations in functional connectivity following traumatic brain injury.

Treating a patient suffering from TBI and associated sleep disturbance with 5-MeO-DMT or a pharmaceutically acceptable salt thereof reduces or eliminates the sleep disturbance and leads to an improvement of the TBI.

The reduction or elimination of sleep disturbance in a patient suffering from TBI is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from TBI occurs not later than about 24 hours after the last administration of 5-MeO-DMT or a pharmaceutically acceptable salt thereof. The reduction or elimination of sleep disturbance in a patient suffering from TBI preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from TBI is observed on day 7 after the last administration of 5-MeO-DMT or a pharmaceutically acceptable salt thereof and preferably persists until at least 14 days, more preferably until at least 28 days.

An improvement in sleep disturbance in a patient suffering from TBI, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from TBI, as reflected by a reduction in the CGI-S score, occurs not later than about 24 hours after the last administration of 5-MeO-DMT or a pharmaceutically acceptable salt thereof. An improvement in sleep disturbance in a patient suffering from TBI, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from TBI, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from TBI is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from TBI as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from TBI as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from TBI as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is one of the most common primary complaints by patients with TBI. An improvement in sleep disturbance will therefore also lead to an improvement of the TBI. Since sleep disturbance furthermore also affects other aspects of TBI, the inventors conclude that the improvement in sleep disturbance, in particular the reduction or elimination of reduced sleep, will additionally contribute to an overall improvement of the TBI.

An improvement of the TBI in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the TBI in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the TBI in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of TBI in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Sleep Disturbance in Patients with HIV Infection

HIV disease is caused by infection with human immunodeficiency virus type-1 (HIV-1). Despite combined antiretroviral therapy, HIV-infected patients develop symptoms of nervous system conditions.

Sleep disturbance among HIV patients is a frequently reported symptom, which is uniquely different from populations with other diseases in terms of patterns and severity. Sleep disturbances are the most prevalent and distressing symptoms experienced, even when HIV disease is well managed.

HIV-related sleep disturbances characterised by ‘a disruption in the amount and quality of sleep that impairs functioning’. Difficulty falling asleep (sleep onset>30 min) and sleep fragmentation (wake after sleep onset >15% of the night) are the most common types of sleep disturbances reported.

Several pathophysiological processes in HIV infection may cause sleep-wake dysregulation. In early infection stages, immunological changes such as expression of sleep-promoting cytokines could mediate sleep disturbances. Long term, chronic immune activation, in addition to side effects of antiretroviral therapy, may impact sleep homeostasis more severely.

To assess sleep disturbancer in patients suffering from sleep disturbance due to HIV Infection, the Pittsburgh Sleep Quality Index (PSQI) is widely used.

Resting state functional connectivity analysis of patients suffering from neurocognitive disorder due to HIV infection, shows that functional connectivity is impaired within and/or between certain regions of the default mode network, the salience network, and the executive network.

Treating a patient suffering from HIV and associated sleep disturbance with 5-MeO-DMT or a pharmaceutically acceptable salt thereof reduces or eliminates the sleep disturbance and leads to an improvement of the HIV.

The reduction or elimination of sleep disturbance in a patient suffering from HIV is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from HIV occurs not later than about 24 hours after the last administration of 5-MeO-DMT or a pharmaceutically acceptable salt thereof. The reduction or elimination of sleep disturbance in a patient suffering from HIV preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from HIV is observed on day 7 after the last administration of 5-MeO-DMT or a pharmaceutically acceptable salt thereof and preferably persists until at least 14 days, more preferably until at least 28 days.

An improvement in sleep disturbance in a patient suffering from HIV, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from HIV, as reflected by a reduction in the CGI-S score, occurs not later than about 24 hours after the last administration of 5-MeO-DMT or a pharmaceutically acceptable salt thereof. An improvement in sleep disturbance in a patient suffering from HIV, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from HIV, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from HIV is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from HIV as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from HIV as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from HIV as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is one of the most common primary complaints by patients with HIV. An improvement in sleep disturbance will therefore also lead to an improvement of the HIV. Since sleep disturbance furthermore also affects other aspects of HIV, the inventors conclude that the improvement in sleep disturbance, in particular the reduction or elimination of reduced sleep, will additionally contribute to an overall improvement of the HIV.

An improvement of the HIV in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the HIV in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the HIV in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of HIV in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

Sleep Disturbance in Patients with Post COVID Condition

Coronavirus disease 2019 (COVID-19) is a contagious disease caused by the severe acute respiratory syndrome coronavirus 2 (SARS-CoV-2).

Post COVID Condition (sometimes referred to as “Long COVID”) is a multisystemic condition comprising often severe symptoms that follow a SARS-CoV-2 infection. It is an often-debilitating illness that occurs in at least 10% of the infections.

The World Health Organization (WHO) defines this condition as a symptom complex which occurs within 3-months after infection, lasts at least 2-months, can fluctuate and for which there is no explanatory explanation there is not attributed to alternative diagnoses.

Post COVID Condition can include a wide range of ongoing health problems; these conditions can last weeks, months, or years and can sometimes result in disability.

Individuals suffering from Post COVID Condition most commonly report typical symptoms such as fatigue, shortness of breath and cognitive disorders such as for example difficulty thinking or concentrating (sometimes referred to as “brain fog”). However, patients may also experience respiratory and heart symptoms, neurological symptoms, for example sleep problems (for example insomnia), and digestive symptoms.

Post COVID Condition patients often experience lingering symptoms, such as anxiety, even after surviving a mild case of the disease. Anxiety in Post COVID Condition can be either a direct result of the SARS-CoV-2 infection or caused by an acute SARS-CoV-2 infection-related stay in hospital, in particular a stay in an intensive care unit.

Individuals suffering from Post COVID Condition induced anxiety often have fears about health and recovery, have stress about being off work and the impact on finances and worry about family and friends also getting ill. Moreover, Post COVID Condition induced anxiety leads to trouble sleeping, difficulty concentrating, difficulty remembering things, changes in mood, flashbacks, breathing dysfunction, chest pain and racing thoughts.

To assess sleep disturbance in patients suffering from sleep disturbance due to Post COVID Condition, the Pittsburgh Sleep Quality Index (PSQI) is widely used.

Functional magnetic resonance imaging of patients suffering from Post COVID Condition reveals alterations within and/or between resting state networks. Most commonly, the default mode network is affected.

Treating a patient suffering from Post COVID Condition and associated sleep disturbance with 5-MeO-DMT or a pharmaceutically acceptable salt thereof reduces or eliminates the sleep disturbance and leads to an improvement of the Post COVID Condition.

The reduction or elimination of sleep disturbance in a patient suffering from Post COVID Condition is observed on day 1, for instance after about 24 hours; on day 7; on day 14; and/or on day 28 after the last administration of 5-MeO-DMT or a pharmaceutically acceptable salt thereof.

The reduction or elimination of sleep disturbance in a patient suffering from Post COVID Condition occurs not later than about 24 hours after the last administration of 5-MeO-DMT or a pharmaceutically acceptable salt thereof. The reduction or elimination of sleep disturbance in a patient suffering from Post COVID Condition preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Post COVID Condition is observed on day 7 after the last administration of 5-MeO-DMT or a pharmaceutically acceptable salt thereof and preferably persists until at least 14 days, more preferably until at least 28 days.

An improvement in sleep disturbance in a patient suffering from Post COVID Condition, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement in sleep disturbance in a patient suffering from Post COVID Condition, as reflected by a reduction in the CGI-S score, occurs not later than about 24 hours after the last administration of 5-MeO-DMT or a pharmaceutically acceptable salt thereof. An improvement in sleep disturbance in a patient suffering from Post COVID Condition, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in sleep disturbance in a patient suffering from Post COVID Condition, as reflected by a reduction in the CGI-S score, is observed on day 7 after the last administration of 5-MeO-DMT or a pharmaceutically acceptable salt thereof and preferably persists until at least 14 days, more preferably until at least 28 days.

A reduction or elimination of sleep disturbance in a patient suffering from Post COVID Condition is reflected by an improvement in the Pittsburgh Sleep Quality Index (PSQI) global score, in particular by a decrease to 5 or below, on day 1, for instance, after about 24 hours; on day 7; on day 14 and/or on day 28 after the last administration of 5-MeO-DMT or a pharmaceutically acceptable salt thereof.

A reduction or elimination of sleep disturbance in a patient suffering from Post COVID Condition as reflected by an improvement in the PSQI global score, in particular by a decrease to 5 or below, occurs not later than about 24 hours after the last administration of 5-MeO-DMT or a pharmaceutically acceptable salt thereof. The reduction or elimination of sleep disturbance in a patient suffering from Post COVID Condition as reflected by an improvement in the PSQI global score, in particular by a decrease to 5 or below, preferably persists until at least 6 days; in particular until at least 14 days; more preferably until at least 28 days after the last administration of 5-MeO-DMT or a pharmaceutically acceptable salt thereof.

In an embodiment, a reduction or elimination of sleep disturbance in a patient suffering from Post COVID Condition as reflected by an improvement in the score of the PSQI, in particular by a decrease to 5 or below, is observed on day 7 after the last administration of 5-MeO-DMT or a pharmaceutically acceptable salt thereof and preferably persists until at least 14 days, more preferably until at least 28 days.

As indicated above, sleep disturbance is one of the most common primary complaints by patients with Post COVID Condition. An improvement in sleep disturbance will therefore also lead to an improvement of the Post COVID Condition. Since sleep disturbance furthermore also affects other aspects of Post COVID Condition, the inventors conclude that the improvement in sleep disturbance, in particular the reduction or elimination of reduced sleep, will additionally contribute to an overall improvement of the Post COVID Condition.

An improvement of the Post COVID Condition in a patient also suffering from associated sleep disturbance, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of the Post COVID Condition in a patient also suffering from associated sleep disturbance, as reflected by a reduction in the CGI-S score, occurs not later than about 24 hours after the last administration of 5-MeO-DMT or a pharmaceutically acceptable salt thereof. An improvement of the Post COVID Condition in a patient also suffering from associated sleep disturbance, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement of Post COVID Condition in a patient also suffering from associated sleep disturbance, as reflected by a reduction in the CGI-S score, is observed on day 7 after the last administration of 5-MeO-DMT or a pharmaceutically acceptable salt thereof and preferably persists until at least 14 days, more preferably until at least 28 days.

### EXAMPLES

The following Examples are listed to aid understanding of the invention and are not intended and should not be construed to limit in any way the invention set forth in the claims which follow thereafter.

### Example 1—5-MeO-DMT Aerosol Generation and Administration

Step 1: A stock solution of 5-MeO-DMT free base in 100% ethanol is prepared in a volumetric flask, so that the target dosage of 5-MeO-DMT free base to be administered via inhalation to the volunteer or patient is contained in a solution volume of 200 μl. Typical target dosages are from 1 mg to 25 mg 5-MeO-DMT. E.g. for a target dosage of 18 mg 5-MeO-DMT, 90 mg of 5-MeO-DMT will be dissolved in 100% ethanol for a final solution volume of 1 ml. Aliquots of the stock solution can then be stored in vials until further use.
Step 2: 200 μl of the solution is transferred to a dosing capsule containing the drip pad (Storz & Bickel, Germany), and then the dosing capsule is closed with its lid.
Step 3: The dosing capsule filled with the 5-MeO-DMT ethanol solution is transferred to the filling chamber of a first Volcano Medic Vaporizer, which has been pre-heated with the temperature set at 55° C. Then the airflow of the vaporizer is switched on for 60 seconds at the pre-set rate of about 12 l/min. The heated air will flow through the dosing capsule, allowing the ethanol to evaporate, with the target dosage of 5-MeO-DMT being left in the capsule, as a thin layer covering the stainless-steel wire mesh. Accurate preparation of the dosing capsule can be confirmed by demonstrating that the final weight increase of the capsule compared to the weight of the empty capsule is about equal to the target dosage of 5-MeO-DMT.
Step 4: The prepared dosing capsule is removed from the filling chamber. It is then transferred to the filling chamber of a second Volcano Medic Vaporizer, which has been pre-heated with the temperature set at 210° C. and the airflow on for at least 5 minutes and then turned off immediately prior to transfer. An inhalation balloon with a valve (Storz & Bickel, Germany) is mounted on the socket of the filling chamber, the filling chamber is closed tightly and immediately afterwards the airflow is switched on for exactly 15 seconds at the pre-set flow rate of about 12 l/min, and then turned off. This will allow the full dose of 5-MeO-DMT to aerosolize and be distributed in approximately 3 liters of air in the inhalation balloon. Accurate aerosolization of the 5-MeO-DMT can be confirmed by demonstrating that the capsule weight has returned to about its initial weight.
Step 5: The balloon is then disconnected from the filling chamber, which automatically closes the valve. After attachment of the mouthpiece to the balloon, the aerosol is ready for immediate administration to the volunteer or patient.
Step 6: To prepare for the administration, the patient is asked to initially perform 1-2 deep inhalations with full exhalations, ending this sequence with a deep exhalation. Then, with the mouthpiece firmly held against the lips, the full and complete volume of the inhalation balloon is inhaled in one inhalation, holding the breath for 10 (±2.5) seconds, followed by a normal exhalation. After completing the inhalation procedure, the patient will be instructed to lie down.

Further details regarding the administration of 5-MeO-DMT by inhalation are disclosed in Example 1 of WO 2020/169850 A1, the contents of which is incorporated herein by reference.

### Example 2—Preparation of 5-MeO-DMT in High Purity

5-MeO-DMT (2.0 g) was dissolved in MTBE (4 mL, 2.0 volumes) at 35 to 40° C. before being cooled to room temperature over 30 minutes. After stirring at room temperature for 50 minutes no crystallisation was observed, therefore, the batch temperature was decreased to 7 to 12° C. over 30 minutes. After stirring at 7 to 12° C. for 10 minutes crystallisation occurred. The batch was subsequently filtered following a 1 hour stir out at 7 to 12° C. After washing with MTBE (1 mL, 0.5 volumes), at 7 to 12° C., the batch was pulled dry under vacuum for 3.5 hours to yield a pale orange solid in 1.02 g (50% recovery). The isolated solid was analysed for purity by HPLC as described in WO 2020/169850 A1. The purity was found to be 99.74% area.

The results from the analysis further indicate that the level of individual impurities was below 0.10% area. Solvent analysis of sample indicated an MTBE level of 17 ppm.

### Example 3—Preparation of 5-MeO-DMT Hydrobromide Salt

5-MeO-DMT HBr was prepared on a 100 mg scale.

5-MeO-DMT free base was combined with isopropyl acetate (10 vols), and the resulting solution of 5-MeO-DMT was heated to 50° C. HBr was charged (1M in ethanol, 1 eq) in one single aliquot. The mixture was held at temperature and equilibrated for 3 hours.

After 1 hour, a suspension had formed. The suspension was finally cooled to room temperature and equilibrated for 18 hours. Solids were isolated by filtration and dried in vacuo at 40° C. for 18 hours.

An off-white crystalline material was obtained.

The salt has a melting point of 174° C. and is characterized by an X-ray diffraction pattern comprising peaks at 14.5°2•±0.2°2•; 16.7°2•±0.2°2•; 17.0°2•±0.2°2•; 20.6°2•±0.2°2•; 20.7°2•±0.2°2•; 21.4°2•±0.2°2•; 24.2°2•±0.2°2•; 24.8°2•±0.2°2•; 25.3°2•±0.2°2•; 27.4°2•±0.2°2•; measured using Cu K• radiation.

### Example 4—Determination of Inhibition Constants for Central 5-HT1A and 5-HT2A Receptors in Post-Mortem Human Brain Membrane Preparations

In this study, the affinity of three psychedelic test compounds (psilocin, DMT and 5-MeO-DMT) for 5-HT1A and 5-HT2A receptors in post-mortem human brain tissue from the hippocampus and frontal cortex, respectively, was determined using the technique of radioligand binding.

Human brain samples were obtained from the Edinburgh Sudden Death Brain Bank. All donors were sudden deaths with no prior history of coma, psychiatric or neurological disorders and under the age of 65 with a post-mortem interval of less than or equal to 72 hours.

### Binding to 5-HT1A Receptors in Post-Mortem Human Hippocampus

Hippocampus was homogenised in ice-cold 0.25 M sucrose (1:30 w/v) using a motor driven Teflon pestle (12 strokes at 120 rpm). Myelin and cell debris were removed by centrifugation at 1,000 g for 10 minutes. The supernatant was stored on ice and the pellet re-homogenised in 0.25 M sucrose (1:15 w/v) and centrifuged at 750 g for 10 minutes. The supernatants were combined and diluted in ice-cold membrane preparation buffer, (1:100 w/v) using a tight-fitting glass/Teflon homogeniser (12 strokes, 800 rpm) and centrifuged at 20,500 g for 10 minutes. The pellet was resuspended in ice-cold membrane preparation buffer and incubated at 37° C. for 10 minutes before being centrifuged at 20,500 g for 10 minutes. The pellet was resuspended and centrifuged a final time to wash the tissue (20,500×g for 10 mins). The resulting pellet was then resuspended in ice-cold assay buffer at a tissue concentration equivalent to 3.125 mg wet weight of tissue/ml. All centrifugations were carried out at 4° C. The membrane preparation buffer consisted of 50 mM Tris-HCl, pH 7.7, 4 mM CaCl₂ and 0.1% ascorbic acid. The assay buffer consisted of 50 mM Tris, pH 7.7, 4 mM CaCl₂, 0.1% ascorbic acid and 10 μM Pargyline.

For saturation binding analysis, hippocampal membranes (400 μl; equivalent 1.25 mg wet weight tissue/tube) was incubated with 50 μl of 0.075-9.6 nM [³H]8-OH-DPAT and either 50 μl of assay buffer (total binding) or 50 μl of 1 μM WAY 100635 (non-specific binding) at 25° C. for 30 minutes. The wash buffer consisted of 50 mM Tris, pH 7.7.

In a displacement assay, hippocampal membranes (400 μl; equivalent 1.25 mg wet weight tissue/tube) were incubated with 50 μl of 0.6 nM [³H]8-OH-DPAT and either 50 μl of assay buffer (total binding) or 50 μl of 1 •M WAY 100635 (non-specific binding) or 50 μl of one of the test compounds in one of ten concentrations between 1 and 10000 nM at 25° C. for 30 minutes.

Membrane bound radioactivity was recovered by filtration under vacuum through Skatron 11731 filters, pre-soaked in 0.5% polyethylenimine (PEI) using a Skatron cell harvester. Filters were rapidly washed with ice-cold wash buffer (wash setting 0, 9, 9) and radioactivity determined by liquid scintillation counting (1 ml Packard MV Gold scintillator).

The concentration of compound required to inhibit 50% of specific binding (IC₅₀) and the Hill Slope were calculated by using non-linear regression. The Kᵢ was calculated using the one-site binding model allowing for ligand depletion.

### Binding to 5-HT2A Receptors in Post-Mortem Human Frontal Cortex

Frontal cortex was homogenised in ice-cold 0.25 M sucrose (1:30 w/v) using a motor driven Teflon pestle (12 strokes at 120 rpm). Myelin and cell debris was removed by centrifugation at 1,000 g for 10 minutes. The supernatant was stored on ice and the pellet re-homogenised in 0.25 M sucrose (1:15 w/v) and centrifuged at 750 g for 10 minutes. The supernatants were combined and diluted in ice-cold 50 mM Tris-HCl assay buffer, pH 7.4 (1:100 w/v), homogenised using a tight-fitting glass/Teflon homogeniser (12 strokes, 800 rpm) and centrifuged at 20,500 g for 10 minutes. The pellet was centrifuged a further two times to wash the tissue (20,500×g for 10 mins). The resulting pellet was then resuspended in ice-cold 50 mM Tris-HCl assay buffer, pH 7.4 at a tissue concentration equivalent to 10 mg wet weight of tissue/ml. All centrifugations were carried out at 4° C.

For saturation binding analysis, frontal cortical membranes (400 μl; equivalent to 4 mg wet weight of tissue/tube) were incubated with 50 μl of 0.00625-0.8 nM [³H]MDL-100,907 and either 50 μl of assay buffer or 50 μl of 10 •M ketanserin (non-specific binding) at 25° C. for 60 minutes. The assay and wash buffer consisted of 50 mM Tris-HCl buffer pH 7.4.

In a displacement assay, frontal cortical membranes (400 μl; equivalent 4 mg wet weight tissue/tube) was incubated with 50 μl of 0.1 nM [3H]MDL-100,907 and either 50 μl of assay buffer (total binding) or 50 μl of 10 •M ketanserin (non-specific binding) or 50 μl of one of the test compounds in one of ten concentrations between 1 and 10000 nM at 25° C. for 60 minutes.

Membrane bound radioactivity was recovered and determined as above. Data analysis was also as above.

### Results

The dissociation constant (K_{d} value) of [³H]8-OH-DPAT for 5-HT1A receptors in hippocampal membranes from post-mortem human brain tissue was determined for each of the three donors. The dissociation constants (K_{d} values) obtained were 0.51, 0.28 and 0.52 nM, respectively.

Mean inhibition constants (Kᵢ values) for psilocin, DMT and 5-MeO-DMT were 48, 38 and 1.80 nM (mean n=3), respectively. All compounds gave Hill slopes approximating to unity, suggesting a one-site binding model.

The dissociation constant (K_{d} values) of [³H]MDL-100,907 for 5-HT2A receptors in frontal cortical membranes from post-mortem human brain tissue was determined for each of the three donors. The dissociation constants (K_{d} values) obtained were 0.11, 0.08 and 0.08 nM, respectively.

Mean inhibition constants (Kᵢ values) for psilocin, DMT and 5-MeO-DMT were 37, 117 and 122 nM (mean n=3), respectively. All compounds gave Hill slopes approximating to unity, suggesting a one-site binding model.

The selectivity ratio of psilocin, DMT and 5-MeO-DMT for 5-HT2A over 5-HT1A receptors was 0.78, 3.1 and 68, respectively.

### Example 5—Toxicological Testing of 5-MeO-DMT

5-MeO-DMT did not induce mutation in four histidine-requiring bacterial strains (TA98, TA100, TA1535 and TA1537) of Salmonella typhimurium, and one tryptophan-requiring strain (WP2 uvrA pKM101) of Escherichia coli. These conditions included treatments at concentrations up to 5000 μg/plate (the maximum recommended concentration according to current regulatory guidelines), in the absence and in the presence of a rat liver metabolic activation system (S-9).

### Example 6—Assessment of the Pharmacokinetics of 5-MeO-DMT and Bufotenine

In order to investigate the pharmacokinetic properties of 5-MeO-DMT, three groups of 8 subjects each were formed. Subjects were administered a single dose of 6 mg; 12 mg or 18 mg 5-MeO-DMT via inhalation. Blood samples were obtained at 1; 2; 4; 7; 10; 15; 20; 30; 45 min and 1; 1.5; 2; 3; 4 hours after administration.

5-MeO-DMT concentrations were determined using LC-MS/MS. PK parameters were generated by algebraic analysis of the concentration versus time plots for each individual. The analysis was carried out using the software Phoenix WinNonlin 6.3.

Median Cmax values obtained for the three groups were 11.85 ng/ml (6 mg group), 22.90 ng/ml (12 mg group) and 38.45 ng/ml (18 mg group).

Table 1 below shows median percentage plasma concentrations relative to Cmax as determined for the time points indicated.

Median percentage plasma concentration relative to Cmax

| | time (min) | | | | | | | | | | | | | |
|---|---|---|---|---|---|---|---|---|---|---|---|---|---|---|
| | 1 | 2 | 4 | 7 | 10 | 15 | 20 | 30 | 45 | 60 | 90 | 120 | 180 | 240 |
| 6 mg | 96 | 98 | 83 | 56 | 40 | 22 | 16 | 10 | 8 | 4 | 2 | 1 | 0 | N.A. |
| 12 mg | 100 | 79 | 59 | 30 | 22 | 12 | 8 | 5 | 3 | 2 | 0 | 0 | 0 | 0 |
| 18 mg | 100 | 79 | 59 | 30 | 22 | 12 | 8 | 5 | 3 | 2 | 0 | 0 | 0 | 0 |

Pharmacokinetic measurements were also carried out for dosing schemes relying on uptitration. Substantially similar results were obtained.

Blood concentrations were also determined for the 5-MeO-DMT metabolite bufotenine. Only in few samples, concentrations were above the lower level of quantification (LLOQ) (25 pg/ml). From 15 min onwards, the bufotenine concentration was always below the LLOQ.

Substantially similar observations were made when subjects receiving an uptitration scheme were included.

### Example 7—Clinical Trial in Patients Suffering from TRD

A Phase 1/2 clinical trial of 5-MeO-DMT, administered via inhalation as described herein, in patients with treatment-resistant major depressive disorder (TRD) has been completed. This trial was designed in two parts. Part A was an open-label, single-arm, single-dose Phase 1 trial with two dose levels (12 mg (n=4) and 18 mg (n=4)). Part B was an open-label, single-arm Phase 2 trial applying an individualized dosing regimen with intra-patient dose escalation with 5-MeO-DMT. Patients (n=8) received at least one and up to three doses of 5-MeO-DMT (6 mg, 12 mg and 18 mg) in a single day, with higher doses only being administered if a peak experience was not achieved at the previously administered dose. The primary endpoint of Part A was to assess the safety and tolerability of single dosing of 5-MeO-DMT in patients with TRD. The primary endpoint of Part B was to assess the effects on the severity of depression, as assessed by the proportion of patients in remission on day seven after dosing, defined as a MADRS total score of less than or equal to 10.

In Part A, 3 of 4 patients in both groups (12 mg and 18 mg) experienced at least one ADR, all of which were mild and resolved spontaneously. No SAEs were reported.

Two of four patients (50%) in the 12 mg group and one of four patients (25%) in the 18 mg group had a MADRS remission on day seven after dosing, and one further patient (25%) in the 18 mg group had a MADRS clinical response on day seven after dosing. The mean MADRS change from baseline at day seven was −21.0 (−65%) in the 12 mg group and −12.8 (−41%) in the 18 mg group.

In Part B, 7 of 8 patients (87.5%) experienced at least one ADR. All ADRs resolved spontaneously. No SAEs were reported.

The primary endpoint was met with seven of eight patients (87.5%) achieving a MADRS remission on day seven (p<0.0001). The mean MADRS change from baseline at day seven was 24.4 (76%).

No clinically significant changes were observed in either Part A or Part B in any of the safety laboratory analyses, vital signs, psychiatric safety assessments or measures of cognitive function.

Results are summarized in the tables below.

TABLE 2-A
Scores recorded against relevant MADRS and BPRS items for patients assigned to
intra-day individualised dosing regimen (IDR). The item scores represent the sum
of the individual patient scores for all patients (n = 8) in the IDR group.
Assessment 2 hours (MADRS) or 3 hours (BPRS) after administration of the last dose.

| | | Baseline | 2/3 | Improvement | Percentage |
|---|---|---|---|---|---|
| Scale item | Scale | IDR | hours | 2/3 hours | improvement |
| Concentration | MADRS | 30 | 11 | 19 | 63.33% |
| Difficulties | | | | | |
| Lassitude | MADRS | 27 | 10 | 17 | 62.96% |
| Inability to Feel | MADRS | 36 | 12 | 24 | 66.67% |
| Inner Tension | MADRS | 26 | 11 | 15 | 57.69% |
| Pessimistic Thoughts | MADRS | 28 | 7 | 21 | 75.00% |
| Suicidal Thoughts | MADRS | 11 | 3 | 8 | 72.72% |
| Guilt Feelings | BPRS | 34 | 14 | 20 | 58.82% |
| Tension | BPRS | 16 | 11 | 5 | 31.25% |
| Anxiety | BPRS | 37 | 19 | 18 | 48.65% |
| Emotional Withdrawal | BPRS | 13 | 8 | 5 | 38.46% |
| Blunted Affect | BPRS | 15 | 11 | 4 | 26.67% |

TABLE 2-B
Scores recorded against relevant MADRS and BPRS items for patients
assigned to intra-day individualised dosing regimen (IDR). The
item scores represent the sum of the individual patient scores
for all patients (n = 8) in the IDR group. Assessment on day 1.

| | | Baseline | | Improvement | Percentage |
|---|---|---|---|---|---|
| Scale item | Scale | IDR | Day 1 | at Day 1 | improvement |
| Reduced Sleep | MADRS | 25 | 12 | 13 | 52.00% |
| Concentration | MADRS | 30 | 1 | 29 | 96.67% |
| Difficulties | | | | | |
| Lassitude | MADRS | 27 | 5 | 22 | 81.48% |
| Inability to Feel | MADRS | 36 | 2 | 34 | 94.44% |
| Inner Tension | MADRS | 26 | 6 | 20 | 76.92% |
| Pessimistic Thoughts | MADRS | 28 | 4 | 24 | 85.71% |
| Suicidal Thoughts | MADRS | 11 | 1 | 10 | 90.91% |
| Guilt Feelings | BPRS | 34 | 11 | 23 | 67.65% |
| Tension | BPRS | 16 | 11 | 5 | 31.25% |
| Anxiety | BPRS | 37 | 16 | 21 | 56.76% |
| Emotional Withdrawal | BPRS | 13 | 8 | 5 | 38.46% |
| Blunted Affect | BPRS | 15 | 8 | 7 | 46.67% |

TABLE 2-C
Scores recorded against relevant MADRS and BPRS items for patients
assigned to intra-day individualised dosing regimen (IDR). The
item scores represent the sum of the individual patient scores
for all patients (n = 8) in the IDR group. Assessment on day 7.

| | | Baseline | | Improvement | Percentage |
|---|---|---|---|---|---|
| Scale item | Scale | IDR | Day 7 | at Day 7 | improvement |
| Reduced Sleep | MADRS | 25 | 9 | 16 | 64.00% |
| Concentration | MADRS | 30 | 9 | 21 | 70.00% |
| Difficulties | | | | | |
| Lassitude | MADRS | 27 | 3 | 24 | 88.89% |
| Inability to Feel | MADRS | 36 | 6 | 30 | 83.33% |
| Inner Tension | MADRS | 26 | 12 | 14 | 53.85% |
| Pessimistic Thoughts | MADRS | 28 | 3 | 25 | 89.29% |
| Suicidal Thoughts | MADRS | 11 | 3 | 8 | 72.73% |
| Guilt Feelings | BPRS | 34 | 10 | 24 | 70.59% |
| Tension | BPRS | 16 | 10 | 6 | 37.50% |
| Anxiety | BPRS | 37 | 17 | 20 | 54.05% |
| Emotional Withdrawal | BPRS | 13 | 8 | 5 | 38.46% |
| Blunted Affect | BPRS | 15 | 8 | 7 | 46.67% |

TABLE 3-A
Scores recorded against relevant MADRS and BPRS items for patients assigned
to 12 mg dosing regimen. The item scores represent the sum of the individual
patient scores for all patients (n = 4) in the 12 mg group. Assessment
2 hours (MADRS) or 3 hours (BPRS) after administration of the dose.

| | | | | Improvement | |
|---|---|---|---|---|---|
| | | Baseline | 2/3 | after 2/3 | Percentage |
| Scale item | Scale | 12 mg | hours | hours | improvement |
| Concentration | MADRS | 16 | 7 | 9 | 56.25% |
| Difficulties | | | | | |
| Lassitude | MADRS | 16 | 10 | 6 | 37.50% |
| Inability to Feel | MADRS | 16 | 9 | 7 | 43.75% |
| Inner Tension | MADRS | 13 | 2 | 11 | 84.62% |
| Pessimistic Thoughts | MADRS | 16 | 8 | 8 | 50.00% |
| Suicidal Thoughts | MADRS | 8 | 3 | 5 | 62.50% |
| Guilt Feelings | BPRS | 18 | 9 | 9 | 50.00% |
| Tension | BPRS | 14 | 9 | 5 | 35.71% |
| Anxiety | BPRS | 25 | 11 | 14 | 56.00% |
| Emotional Withdrawal | BPRS | 13 | 11 | 2 | 15.38% |
| Blunted Affect | BPRS | 11 | 8 | 3 | 27.27% |

TABLE 3-B
Scores recorded against relevant MADRS and BPRS items for patients assigned to
12 mg dosing regimen. The item scores represent the sum of the individual patient
scores for all patients (n = 4) in the 12 mg group. Assessment on day 1.

| | | Baseline | | Improvement | Percentage |
|---|---|---|---|---|---|
| Scale item | Scale | 12 mg | Day 1 | at Day 1 | improvement |
| Reduced Sleep | MADRS | 12 | 10 | 2 | 16.67% |
| Concentration | MADRS | 16 | 2 | 14 | 87.50% |
| Difficulties | | | | | |
| Lassitude | MADRS | 16 | 0 | 16 | 100.00% |
| Inability to Feel | MADRS | 16 | 1 | 15 | 93.75% |
| Inner Tension | MADRS | 13 | 3 | 10 | 76.92% |
| Pessimistic Thoughts | MADRS | 16 | 7 | 9 | 56.25% |
| Suicidal Thoughts | MADRS | 8 | 5 | 3 | 37.50% |
| Guilt Feelings | BPRS | 18 | 5 | 13 | 72.22% |
| Tension | BPRS | 14 | 6 | 8 | 57.14% |
| Anxiety | BPRS | 25 | 6 | 19 | 76.00% |
| Emotional Withdrawal | BPRS | 13 | 8 | 5 | 38.46% |
| Blunted Affect | BPRS | 11 | 6 | 5 | 45.45% |

TABLE 3-C
Scores recorded against relevant MADRS and BPRS items for patients assigned to
12 mg dosing regimen. The item scores represent the sum of the individual patient
scores for all patients (n = 4) in the 12 mg group. Assessment on day 7.

| | | Baseline | | Improvement | Percentage |
|---|---|---|---|---|---|
| Scale item | Scale | 12 mg | Day 7 | at Day 7 | improvement |
| Reduced Sleep | MADRS | 12 | 6 | 6 | 50.00% |
| Concentration | MADRS | 16 | 3 | 13 | 81.25% |
| Difficulties | | | | | |
| Lassitude | MADRS | 16 | 3 | 13 | 81.25% |
| Inability to Feel | MADRS | 16 | 1 | 15 | 93.75% |
| Inner Tension | MADRS | 13 | 5 | 8 | 61.54% |
| Pessimistic Thoughts | MADRS | 16 | 8 | 8 | 50.00% |
| Suicidal Thoughts | MADRS | 8 | 7 | 1 | 12.50% |
| Guilt Feelings | BPRS | 18 | 5 | 13 | 72.22% |
| Tension | BPRS | 14 | 6 | 8 | 57.14% |
| Anxiety | BPRS | 25 | 6 | 19 | 76.00% |
| Emotional Withdrawal | BPRS | 13 | 6 | 7 | 53.85% |
| Blunted Affect | BPRS | 11 | 5 | 6 | 54.55% |

### Example 8—Clinical Trial of 5-MeO-DMT Administered Via Inhalation to Patients with Postpartum Depression

The single-arm, open-label clinical trial will involve 15 adult female patients with clinically diagnosed postpartum depression (PPD).

The patients will receive a single-day individualized 5-MeO-DMT dosing regimen via inhalation after vaporization.

More in particular, the patients will receive up to three doses of 5-MeO-DMT on Day 0: 6 mg, 12 mg, and 18 mg.
1. All patients will receive an initial dose of 6 mg 5-MeO-DMT.
2. The second dose (12 mg) will only be administered if:
   a. A peak experience (total score of •75) has not been achieved following the 6 mg dose, and
   b. The 6 mg dose was safe and well-tolerated according to the investigator,
   c. Any psychoactive effects (PsE) of the prior dose have subsided, and
   d. Pre-dose vital parameters and forced expiratory volume in one second (FEV1) are in normal range, or if outside of the normal range, are not clinically significant according to the investigator.
3. Similarly, a third dose (18 mg) will only be administered if:
   a. A peak experience (total score of •75) has not been achieved following the 12 mg dose, and
   b. The 12 mg dose was safe and well-tolerated according to the investigator, and
   c. Any PsE of the prior dose have subsided, and
   d. Pre-dose vital parameters and forced expiratory volume in one second (FEV1) are in normal range, or if outside of the normal range, are not clinically significant according to the investigator.

The patients will be assessed for a peak psychedelic experience (based on a patient-scored visual analogue scale, the PE scale), sedation, and other endpoints after dosing. Follow-up visits are planned for Day 1, and Day 7 after the dosing day.

The following criteria must be met by all patients considered for clinical trial participation:
1. Is female and in the age range between 18 and 45 years (inclusive) at screening.
2. Has a body mass index (BMI) in the range of 18.5 and 35 kg/m² (inclusive) at screening.
3. Meets the trial criteria for PPD as assessed by a trial psychiatrist or registered psychologist:
   a. Diagnosis of Major Depressive Disorder without psychotic features, confirmed by the Mini-International Neuropsychiatric Interview (MINI), with peri-partum onset that began no earlier than gestation and no later than the first 4 weeks postpartum.
   b. Has a Montgomery-Åsberg Depression Rating Scale (MADRS) total score of equal to or greater than 28 at screening and pre-dose on Day 0.
6. Must have either ceased lactating at screening; or, if still lactating or actively breast feeding at screening, must agree to temporarily cease breastfeeding from just prior to receiving study drug on Day 0 through 24 hours post last dose, and to pump and discard all breastmilk during those 24 hours as needed, but need to include a pump/discard at 2.5 hours post last dose and 24 hours post last dose prior to reinitiating breastfeeding.
4. Must agree to remain completely abstinent (complete avoidance of heterosexual intercourse) or use a highly effective (failure rate<1%), medically accepted contraceptive method for 30 days prior to dosing and for 90 days after 5-MeO-DMT dosing. Patients must have a negative pregnancy test at screening and on the pre-test day (Day −1).
5. Is willing to delay start of other antidepressant or anxiety medication until after the end of the trial at Day 7 and agrees to keep any psychotherapy unchanged during the trial.

A potential patient who meets any of the following key exclusion criteria will be excluded from participation in this trial:
1. Has, based on history, psychiatric assessment, and evaluation of the MINI, a current or prior diagnosis of bipolar disorder, a manic or hypomanic episode, a psychotic disorder, Major Depressive Disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed bipolar disorder, psychotic disorder or other mood disorder (including MDD) with psychotic features.
3. Is in the judgement of a trial psychiatrist or registered psychologist, at significant risk for suicide based on history, psychiatric assessment, and evaluation of suicidal ideation and suicidal behaviour based on the Columbia-Suicide Severity Rating Scale (C-SSRS).
4. Has taken anti-depressive medication within 14 days or 5 half-lives (whichever is longer) prior to dosing (exception: within the last 5 weeks in the case of fluoxetine).
5. Has taken any other medication with monoamine oxidase inhibitor (MAOI) activity within 14 days or 5 half-lives (whichever is longer) prior to dosing.
6. Has previously experienced a significant adverse reaction to a hallucinogenic or psychedelic drug (e.g., psilocybin, Psilocybe spp. mushrooms, 5-MeO-DMT, DMT, ayahuasca, LSD, mescaline) according to the investigator's judgment.
7. Has known allergies or hypersensitivity or any other contraindication to 5-MeO-DMT.
8. Has any current or past clinically significant condition (e.g., severe infection, pulmonary disease, •uncontrolled•hypertension, new onset of hypertensive disorders of pregnancy during pregnancy or in the postnatal period (e.g., gestational hypertension, pre-eclampsia-eclampsia, superimposed pre-eclampsia), uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that renders the patient unsuitable for the trial according to the investigator's•judgment.
9. Takes any medication or other substance that renders the patient unsuitable for the trial according to the investigator's judgment.
10. Has a clinically significant abnormality in physical examination, vital signs, ECG, or clinical laboratory parameters which renders the patient unsuitable for the trial according to the investigator's judgment.
11. Patient who has a positive pregnancy test at screening or on the pre-test day (Day −1), is pregnant, or plans to become pregnant during the course of the trial and up to 90 days after 5-MeO-DMT dosing.
12. Patients with DSM-5 drug or alcohol use disorder within 6 months prior to screening.

The primary objective of the trial is to determine the onset and 7-day durability of anti-depressive effects of a single-day individualized dosing regimen of 6 mg, 12 mg and 18 mg of 5-MeO-DMT in adult, female patients with PPD.

Secondary objectives are to determine the anti-depressive effects; the anti-anxiety effects; the effects on maternal behavior; the safety and tolerability; the intensity and duration of psychoactive effects (PsE); the impact on cognitive outcome of a single-day individualized dosing regimen of 6 mg, 12 mg and 18 mg of 5-MeO-DMT in adult, female patients with PPD.

An exploratory objective is to determine in breastmilk, blood and urine the amount of 5-MeO-DMT and metabolites, bufotenine and 5-methoxyindole-3-acetic acid (5-MIAA), measured by LC/MS/MS (metabolite identification screening may be performed, as required), following dose administration of a single-day IDR of 6 mg, 12 mg and 18 mg of 5-MeO-DMT in adult, female patients with PPD.

The primary endpoint of the study is the evaluation of the anti-depressive effects of 5-MeO-DMT by the change from baseline in MADRS assessed at Day 7.

Secondary endpoints include the anti-depressive effects of 5-MeO-DMT evaluated by
The anti-depressive effects of 5-MeO-DMT evaluated by:
   The proportion of patients in remission (MADRS•10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (•50% reduction from baseline in MADRS total score) at 2 hours after the final study drug dosing on Day 0, and at Day 1 and Day 7;
   Change from baseline in Clinical Global Impression-Severity scale (CGI-S) 2 hours after final study drug dosing on Day 0, and at Day 1 and Day 7;
Effects on maternal behaviour as assessed by the change from baseline in the Barkin Index of Maternal Functioning (BIMF) total and sub-scale scores to Day 7;
Exposure of 5-MeO-DMT and bufotenine in breastmilk obtained at the pre-test day (Day −1), 1 hour after last study drug dosing, at discharge, in the evening of Day 0, and on Day 1 and on Day 7;
Exposure of 5-MeO-DMT and bufotenine in blood obtained at the pre-test day (Day −1), 1 hour after last study drug dosing, at discharge, on Day 1 and on Day 7;
The safety and tolerability of 5-MeO-DMT evaluated by:
   Reporting of treatment-emergent adverse events (TEAEs);
   Clinically significant changes from baseline in ECG, vital signs, safety laboratory assessments, peak flow respirometry;
   Assessment of sedation (Modified Observer's Assessment of Alertness and Sedation scale [MOAA/S]) following each dose (when the PsE has subsided and 60 minutes after each study drug dosing) and as part of the discharge evaluation on Day 0;
   Change from baseline in Clinician Administered Dissociative States Scale (CADSS) assessed as part of the discharge evaluation on Day 0 and at Day 1 and Day 7;
   Change from baseline in Brief Psychiatric Rating Scale (BPRS) assessed as part of the discharge evaluation on Day 0, and at Day 1 and Day 7;
   Change from baseline in C-SSRS assessed as part of the discharge evaluation on Day 0, and at Day 1 and Day 7;
   Change from baseline in YMRS assessed as part of the discharge evaluation on Day 0, and at Day 1 and Day 7;
The PsE experienced by the patients as reported 30 to 60 minutes after each dosing, when the PsE has subsided:
   PsE assessment using the peak experience (PE) scale to assess the achievement of a PE (PE scale total score •75);
   Challenging Experience Questionnaire (CEQ);
   Mystical Experience Questionnaire (MEQ-30);
Duration of the PsE defined as the time from study drug dosing to the time when the PsE have subsided (investigator- and patient-scored), completed 30 to 60 minutes after each dosing;

One patient with postpartum depression diagnosed by a psychiatrist has, so far, been recruited into the clinical trial. Diagnosis was Major Depressive Disorder without psychotic features, confirmed by the Mini-International Neuropsychiatric Interview (MINI) (v7.0.2), with peri-partum onset that began no earlier than gestation and no later than the first 4 weeks postpartum. The patient was diagnosed with postpartum depression after giving birth to her third child. The patient completed all planned visit days. The inhalation procedure was adequately performed by the patient and was well tolerated with no inhalation-related adverse events.

### Results

Except for a temporary, clinically non-relevant increase in heart rate and blood pressure shortly after administration of 5-MeO-DMT, no other noteworthy changes in vital parameters occurred. Assessments of ECG (at 3 hours after administration) and safety laboratory analyses (at 7 days), CADSS (at 3 hours, 1 day and 7 days) were unremarkable. The few reported adverse events (cramping left abdominal pain and headache, both on Day 0) were mild, short-lasting and resolved spontaneously by the end of the study.

With regard to the intensity of the psychedelic experience, the recorded PES score achieved upon exposure to a nominal dose of 6 mg was 17.3. This score indicated the need to proceed to the administration of a subsequent, higher dose of 12 mg, per the design of the individualised dosing regimen. The PES score achieved for this dose was 85.7 and, being •75, indicated the occurrence of a peak psychedelic experience and the completion of the IDR for this patient.

Significantly, the patient reported a major improvement in her depressive symptoms as assessed by MADRS at the earliest assessment timepoint of 2 hours after drug administration, with the effect being maintained over time (Table 4). The patient also fulfilled standard criteria for MADRS response (at least 50% improvement from baseline) and MADRS remission (MADRS total score equal or less than 10).

TABLE 4
MADRS/BPRS scores table

| | | 2hr | | | | | |
|---|---|---|---|---|---|---|---|
| | | (M ADRS); | | | Improvement | | |
| | Base- | Discharge | | | 2 hr; | | |
| Scale item | line | (BPRS) | Day 1 | Day 7 | Discharge | Day 1 | Day 7 |
| M ADRS | | | | | | | |
| Reported sadness | 4 | 0 | 0 | 0 | 4 | 4 | 4 |
| Apparent sadness | 5 | 0 | 0 | 0 | 5 | 5 | 5 |
| Inner Tension | 5 | 0 | 0 | 1 | 5 | 5 | 4 |
| Reduced Sleep | 5 | 5 | 0 | 0 | 0 | 5 | 5 |
| Reduced appetite | 0 | 0 | 0 | 0 | 0 | 0 | 0 |
| Concentration Difficulties | 0 | 0 | 0 | 0 | 0 | 0 | 0 |
| Lassitude | 3 | 0 | 0 | 0 | 3 | 3 | 3 |
| Inability to Feel | 3 | 0 | 0 | 0 | 3 | 3 | 3 |
| Pessimistic Thoughts | 4 | 0 | 0 | 0 | 4 | 4 | 4 |
| Suicidal Thoughts | 0 | 0 | 0 | 0 | 0 | 0 | 0 |
| Total M ADRS | 29 | 5 | 0 | 1 | 24 | 29 | 28 |
| BPRS | | | | | | | |
| Somatic Concern | 6 | 1 | 1 | 1 | 5 | 5 | 5 |
| Anxiety | 7 | 1 | 1 | 1 | 6 | 6 | 6 |
| Emotional withdrawal | 5 | 1 | 1 | 1 | 4 | 4 | 4 |
| Conceptual disorganization | 1 | 1 | 1 | 1 | 0 | 0 | 0 |
| Guilt feelings | 6 | 1 | 1 | 1 | 5 | 5 | 5 |
| Tension | 6 | 1 | 1 | 1 | 5 | 5 | 5 |
| Mannerisms and Posturing | 1 | 1 | 1 | 1 | 0 | 0 | 0 |
| Grandiosity | 1 | 1 | 1 | 1 | 0 | 0 | 0 |
| Depressive Mood | 6 | 1 | 1 | 1 | 5 | 5 | 5 |
| Hostility | 1 | 1 | 1 | 1 | 0 | 0 | 0 |
| Suspiciousness | 1 | 1 | 1 | 1 | 0 | 0 | 0 |
| Hallucinatory behaviour | 1 | 1 | 1 | 1 | 0 | 0 | 0 |
| Motor retardation | 1 | 1 | 1 | 1 | 0 | 0 | 0 |
| Uncooperativeness | 1 | 1 | 1 | 1 | 0 | 0 | 0 |
| Unusual Thought Content | 1 | 1 | 1 | 1 | 0 | 0 | 0 |
| Blunted affect | 1 | 1 | 1 | 1 | 0 | 0 | 0 |
| Excitement | 2 | 1 | 1 | 1 | 1 | 1 | 1 |
| Disorientation | 1 | 1 | 1 | 1 | 0 | 0 | 0 |

Significant improvements were noted for several MADRS items in particular. The items are outlined in Table 4. While the patient's baseline scores for some items reflected absence of the symptom (reduced appetite, concentration difficulties, suicidal thoughts), items with scores reflecting severe symptoms (e.g., reduced sleep, inner tension) saw remarkable improvement.

Similarly, improvements were seen in several BPRS items, including Somatic Concerns, Anxiety, Emotional withdrawal, Guilt feelings and Tension.

Additionally, improvements in maternal functioning were evidenced by improvements in the BIMF score recorded at Day 7, as outlined in Table 5, with the total score improving by 14% from 92 to 105 (out of a possible total of 120).

Several functional domains of maternal function were also assessed, as defined by Barkin et al. The improvements in each functional domain are outlined in more detail in Table 6.

Here, noteworthy improvements in self-care, psychological well-being and management were achieved, with percentage improvements ranging from 18% (management) to (44%),% (self-care). These improvements reinforce the relationship between improvement in depressive items, as assessed by the MADRS, and improvements in maternal functioning.

It is noted that the patient scored comparatively high already before treatment. In some functional domains, the score was at the maximum value, or close to it (see Table 6), so that the scope for improvement by therapy was limited.

TABLE 5
BIMF scores table

| No. | BIM F item | Day 0 | Day 7 | Δ (Day 7) |
|---|---|---|---|---|
| 1 | I am a good mother | 6 | 6 | 0 |
| 2 | I feel rested | 0 | 2 | 2 |
| 3 | I am comfortable with the way I've chosen to feed my | 6 | 6 | 0 |
| | baby (either bottle or breast, or both) | | | |
| 4 | My baby and I understand each other | 5 | 5 | 0 |
| 5 | I am able to relax and enjoy time with my baby | 5 | 6 | 1 |
| 6 | There are people in my life that I can trust to care for | 6 | 6 | 0 |
| | my baby when I need a break | | | |
| 7 | I am comfortable allowing a trusted friend or relative | 6 | 6 | 0 |
| | to care for my baby (can include baby's father or | | | |
| | partner) | | | |
| 8 | I am getting enough adult interaction | 6 | 6 | 0 |
| 9 | I am getting enough encouragement from other | 5 | 6 | 1 |
| | people | | | |
| 10 | I trust my own feelings (instincts) when it comes to | 5 | 6 | 1 |
| | taking care of my baby | | | |
| 11 | I take a little time each week to do something for | 5 | 6 | 1 |
| | myself | | | |
| 12 | I am taking good care of my baby's physical needs | 6 | 6 | 0 |
| | (feedings, changing diapers, doctor's appointments) | | | |
| 13 | I am taking good care of my physical needs (eating, | 4 | 5 | 1 |
| | showering, etc) | | | |
| 14 | I make good decisions about my baby's health and | 6 | 6 | 0 |
| | well being | | | |
| 15 | My baby and I are getting into a routine | 5 | 6 | 1 |
| 16 | I worry about how other people judge me (as a | 3 | 5 | 2 |
| | mother) | | | |
| 17 | I am able to take care of my baby and my other | 5 | 5 | 0 |
| | responsibilities | | | |
| 18 | Anxiety or worry often interferes with my mothering | 2 | 5 | 3 |
| | ability | | | |
| 19 | As time goes on, I am getting better at taking care of | 0 | 1 | 1 |
| | my baby | | | |
| 20 | I am satisfied with the job I am doing as a new mother | 6 | 5 | −1 |
| | TOTAL | 92 | 105 | 13 |

TABLE 6
BIMF functional domain scores table

| | | Score | | | | |
|---|---|---|---|---|---|---|
| Functional | Related BIM F | Maximum | Day | Day | Δ (Day | |
| Domain | Items | Possible | 0 | 7 | 7) | % Improvement |
| Self Care | 2, 11, 13 | 18 | 9 | 13 | 4 | 44.44% |
| Infant Care | 12, 14 | 12 | 12 | 12 | 0 | 0.00% |
| Mother-Child | 4, 5, 15 | 18 | 15 | 17 | 2 | 13.33% |
| Interaction | | | | | | |
| Psychological | 1, 2, 3, 5, 7, 10, | 60 | 44 | 53 | 9 | 20.45% |
| Well-being | 11, 16, 18, 20 | | | | | |
| Social Support | 6, 8, 9 | 18 | 17 | 18 | 1 | 5.88% |
| Management | 7, 11, 13, 14, 17, 18 | 36 | 28 | 33 | 5 | 17.86% |
| Adjustment | 17, 19 | 12 | 5 | 6 | 1 | 20.00% |

### SUMMARY AND CONCLUSIONS

A. An individualised dosing regimen of 6 mg 5-MeO-DMT, followed by 12 mg 5-MeO-DMT administered via inhalation was well tolerated and induced an astonishing and very significant clinical response in a patient formally diagnosed with postpartum depression.
B. The clinical response occurs rapidly within 2 hours after 5-MeO-DMT administration. Such rapid onset is unusual and has not been seen with conventional classes of antidepressants, including tricyclic antidepressants, monoamine oxidase inhibitors, selective serotonin-reuptake inhibitors (SSRIs), serotonin-norepinephrine reuptake inhibitors (SNRIs), and others, which generally take 4 to 6 weeks to show their effect.
C. The patient experienced a clinical remission within 2 hours after 5-MeO-DMT administration according to the IDR. This is highly superior to any approved therapy for postpartum depression, and also to all previously tested psychedelic agents.
D. A significant clinical response was sustained over the 7-day follow-up period, although 5-MeO-DMT was only given once and is no longer efficaciously present in the body during this time frame (see pharmacokinetic data in Example 6 above). This observation supports the superior clinical profile of 5-MeO-DMT and allows for convenient administration intervals.
E. In addition to anti-depressive effects, endpoints assessing other symptoms (such as somatic concerns, emotional withdrawal, anxiety, guilt and tension) were positively impacted, supporting the use of 5-MeO-DMT in patients with other mental diseases.
F. In addition to anti-depressive effects, endpoints assessing maternal functioning, as assessed using the BIMF, such as self-care, psychological well-being and management, were positively impacted. This supports additional benefits of 5-MeO-DMT to patients suffering from PPD beyond improvement in their core depressive symptoms.

The highlighted aspects show that 5-MeO-DMT has a significantly improved efficacy profile compared to approved pharmacological therapies for postpartum depression and to all previously tested psychedelic agents, when used according to the present invention.

Together with the short duration of the acute psychedelic effects and the favourable safety profile, these data show that the technical problem to provide an improved psychoactive therapy in a patient with a postpartum depression is solved by the present invention.

### Example 9—Clinical Trial of 5-MeO-DMT Administered Via Inhalation to Patients with Bipolar II Disorder

The single-arm, open-label clinical trial will involve 15 adult patients with bipolar II disorder and a current major depressive episode.

Patients who are currently taking anti-depressive medication need to discontinue or taper over time such medication.

The patients will receive a single-day individualized 5-MeO-DMT dosing regimen via inhalation after vaporization.

More in particular, the patients will receive up to three doses of 5-MeO-DMT on the administration day (Day 0): 6 mg, 12 mg, and 18 mg.
1. All patients will receive an initial dose of 6 mg 5-MeO-DMT.
2. The second dose (12 mg) will only be administered if:
   a. A peak experience (PES total score of •75) has not been achieved following the 6 mg dose, and
   b. The 6 mg dose was safe and well-tolerated.
3. Similarly, a third dose (18 mg) will only be administered if:
   a. A peak experience (PES total score of •75) has not been achieved following the 12 mg dose, and
   b. The 12 mg dose was safe and well-tolerated.

The patients will be assessed for a peak psychedelic experience based on the patient-scored PES described above, sedation and other endpoints after dosing. Follow-up visits are planned for Day 1, and Day 7 after the dosing day.

Selection of patients is based on the following key inclusion criteria:
1. Understands the nature of the clinical trial and has provided signed and dated written informed consent in accordance with local regulations before the conduct of any trial-related procedures.
2. Is male or female and in the age range between 18 and 64 years (inclusive) at screening.
3. Meets the trial criteria for bipolar II disorder and is experiencing a major depressive episode, as assessed by a trial psychiatrist or registered clinical psychologist:
   a. Meets the Diagnostic and Statistical Manual of Mental Disorders (DSM-5) diagnostic criteria for bipolar II disorder with a current major depressive disorder episode confirmed by the Mini-International Neuropsychiatric Interview (MINI);
   b. Has a Montgomery-Åsberg Depression Rating Scale (MADRS) total score of equal to or greater than 24 at screening and prior to first dose on Day 0;
4. Has a Young Mania Rating Scale (YMRS) total score less than or equal to 8 at screening and prior to first dose on Day 0;
5. Agrees to keep any psychotherapy unchanged, and not initiate any new psychoactive medications during the course of the trial.
6. Female patients must be either surgically sterile (hysterectomy, tubal ligation, or bilateral oophorectomy (6 months prior to screening)) or postmenopausal with amenorrhea for the last 2 years or remain completely abstinent (complete avoidance of heterosexual intercourse) or use a highly effective (failure rate<1%) medically accepted contraceptive method, including, but not limited to, bilateral tubal ligation/occlusion, hormone contraceptives that inhibit ovulation, intrauterine device (including hormone-releasing intrauterine device/systems) for 30 days before and 90 days after 5-MeO-DMT dosing and must have a negative serum pregnancy test at screening and a negative urine pregnancy test on the pre-test day (Day −1).
7. Male patients must use prophylactic contraception (i.e., condom with spermicide or abstinence) and must not donate sperm for 30 days after 5-MeO-DMT dosing.

A potential patient who meets any of the following key exclusion criteria will be excluded from participation in this trial:
1. Has, based on history, psychiatric assessment, and evaluation of the MINI, a current or prior diagnosis of bipolar I disorder, a manic episode, a psychotic disorder, major depressive disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigators judgment.
2. Has one or more first or second degree relatives with a current or previously diagnosed psychotic disorder, bipolar I disorder, or MDD with psychotic features.
3. Has significant suicide risk as defined by: (a) suicidal ideation as endorsed on items 4 or 5 on the C-SSRS within the past year, during the screening period, or at Baseline; or (b) suicidal behaviours within the past year; or (c)•clinical assessment of significant suicidal risk during clinical interview; or (d) non-suicidal self-injury within the past year.
4. Has taken anti-depressive medication within 7 days or 5 half-lives (whichever is longer) prior to dosing (exception: within the last 5 weeks in the case of fluoxetine).
5. Has taken a medication with monoamine oxidase inhibitor (MAOI) activity within 14 days or 5 half-lives (whichever is longer) prior to dosing.
6. Has taken mood stabilizer therapy (e.g., lamotrigine, valproate, atypical antipsychotics) within 14 days (28 days for lithium) or 5 half-lives (whichever is longer) prior to dosing or takes mood stabilizer therapy at screening or is expected to require mood stabilizer therapy during the study (as per the investigator's judgment).
7. Has previously experienced a significant adverse reaction to a hallucinogenic or psychedelic drug according to the investigator's judgment.
8. Has known allergies or hypersensitivity or any other contraindication to 5-MeO-DMT.
9. Has any current or past clinically significant condition (e.g., severe infection, severe pulmonary disease, •uncontrolled•hypertension, uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that may interfere with the interpretation of the trial results, constitutes a health risk for the patient, or that otherwise renders the patient unsuitable for the trial according to the investigator's judgment.
10. Takes any medication or other substance that renders the patient unsuitable for the trial according to the investigators judgment.
11. Has a clinically significant abnormality in physical examination, vital signs, electrocardiogram (ECG), or clinical laboratory parameters which renders the patient unsuitable for the trial according to the investigators judgment.
12. Female patient who has a positive pregnancy test at screening or on the pre-test day (Day −1), is pregnant or lactating, or plans to become pregnant during the course of the trial and up to 30 days after 5-MeO-DMT dosing.
13. Patients with DSM-5, alcohol or substance use disorder (excluding tobacco or caffeine use disorders) within 6 months prior to screening.

The primary objective of the trial is to determine the onset and durability of anti-depressive effects of a single-day individualized dosing regimen of 6 mg, 12 mg and 18 mg of 5-MeO-DMT in patients with bipolar II disorder and a current major episode of depression. Secondary objectives are to determine the effect on depressive symptoms and global clinical status; the safety and tolerability; the intensity and duration of psychoactive effects (PsE); the impact on sleep quality; the impact on cognitive outcomes of a single-day individualized dosing regimen of 6 mg, 12 mg and 18 mg of 5-MeO-DMT in patients with bipolar II disorder and a current major episode of depression.

The primary endpoint of the study is the evaluation of the anti-depressive effects of 5-MeO-DMT by the change from baseline in MADRS assessed at Day 7.

Secondary endpoints include:
The anti-depressive effects of 5-MeO-DMT administered via inhalation evaluated by:
   The proportion of patients in remission (MADRS •10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (•50% reduction from baseline in MADRS total score) at 2 hours after the final study drug dosing on Day 0, and at Day 1 and Day 7;
   Change from baseline in CGI-S at 2 hours after the final study drug dosing on Day 0, and at Day 1 and Day 7.
   Change from baseline in BDRS at Day 1 and Day 7
The safety and tolerability of 5-MeO-DMT administered via inhalation evaluated by:
   Reporting of treatment-emergent adverse events (TEAEs);
   Clinically significant changes from baseline in ECG, vital signs, safety laboratory assessments and spirometry assessments;
   Assessment of sedation (Modified Observer's Assessment of Alertness and Sedation scale [MOAA/S]) following each dose (when the PsE have subsided and 60 minutes after each study drug dosing) and as part of the discharge evaluation on Day 0;
   The incidence of adverse events (AEs) of mania or hypomania (as assessed using the DSM-5 criteria for mania/hypomania);
   Change from baseline in YMRS assessed as part of the discharge evaluation on Day 0 and at Day 1 and Day 7;
   Change from baseline in Clinician Administered Dissociative States Scale (CADSS) assessed as part of the discharge evaluation on Day 0 and at Day 1 and Day 7;
   Assessment of patient discharge readiness at discharge on Day 0 using the Clinical Assessment of Discharge Readiness (CADR);
   Change from baseline in Brief Psychiatric Rating Scale (BPRS) assessed as part of the discharge evaluation on Day 0, and at Day 1 and Day 7;
   C-SSRS categorization based on the Columbia Classification Algorithm of Suicide Assessment (C-CASA).
The PsE experienced by the patients as reported 30 to 60 minutes after each dosing, when the PsE has subsided:
   PsE assessment using the peak experience (PE) Scale (PES) to assess the achievement of a PE (PES total score •75);
   Challenging Experience Questionnaire (CEQ);
   Mystical Experience Questionnaire (MEQ-30).
Duration of the PsE, defined as the time from study drug dosing to the time when the PsE have subsided, completed 30 to 60 0 minutes after each dosing.
The impact on sleep quality as evaluated by change from pre-test day (Day −1) to Day 1 and to Day 7 in the Pittsburgh Sleep Quality Index (PSQI).
The impact on cognitive outcomes as evaluated by change from the pre-test day (Day −1) to discharge on Day 0, to Day 1 and to Day 7 in:
   Rapid visual information processing (RVP) test;
   Verbal recognition memory (VRM) test;
   Spatial working memory (SWM) test;
   Digit symbol substitution test (DSST).

### Example 10—Clinical Trial of 5-MeO-DMT Administered Via Intravenous Injection to Patients with Bipolar II Disorder—Prophetic Example

The clinical trial will involve adult patients with bipolar II disorder and a current major depressive episode.

Patients who are currently taking anti-depressive medication need to discontinue or taper over time such medication.

The patients will receive a single-day individualized 5-MeO-DMT dosing regimen by intravenous injection. The 5-MeO-DMT will be provided in the form of its hydrobromide salt and a formulation for intravenous injection. It is understood that the dosage amounts for 5-MeO-DMT mentioned below relate to the weight amount of the free base and the dosage amounts for the hydrobromide salt of 5-MeO-DMT can be calculated assuming that equimolar amounts are used.

More in particular, the patients will receive up to three doses of 5-MeO-DMT on the administration day (Day 0): 2 mg, 5 mg, and 8 mg.
1. All patients will receive an initial dose of 2 mg 5-MeO-DMT.
2. The second dose (5 mg) will only be administered if:
   a. A peak experience (PES total score of •75) has not been achieved following the 2 mg dose, and
   b. The 2 mg dose was safe and well-tolerated.
3. Similarly, a third dose (8 mg) will only be administered if:
   a. A peak experience (PES total score of •75) has not been achieved following the 5 mg dose, and
   b. The 5 mg dose was safe and well-tolerated.

The patients will be assessed for a peak psychedelic experience based on the patient-scored PES described above, sedation and other endpoints after dosing. Follow-up visits are planned for Day 1, and Day 7 after the dosing day.

Selection of patients is based on the following key inclusion criteria:
1. Understands the nature of the clinical trial and has provided signed and dated written informed consent in accordance with local regulations before the conduct of any trial-related procedures.
2. Is male or female and in the age range between 18 and 64 years (inclusive) at screening.
3. Meets the trial criteria for bipolar II disorder and is experiencing a major depressive episode, as assessed by a trial psychiatrist or registered clinical psychologist:
   a. Meets the Diagnostic and Statistical Manual of Mental Disorders (DSM-5) diagnostic criteria for bipolar II disorder with a current major depressive disorder episode confirmed by the Mini-International Neuropsychiatric Interview (MINI);
   b. Has a Montgomery-Åsberg Depression Rating Scale (MADRS) total score of equal to or greater than 24 at screening and prior to first dose on Day 0;
5. Has a Young Mania Rating Scale (YMRS) total score less than or equal to 8 at screening and prior to first dose on Day 0;
6. Agrees to keep any psychotherapy unchanged, and not initiate any new psychoactive medications during the course of the trial.
7. Female patients must be either surgically sterile (hysterectomy, tubal ligation, or bilateral oophorectomy (6 months prior to screening)) or postmenopausal with amenorrhea for the last 2 years or remain completely abstinent (complete avoidance of heterosexual intercourse) or use a highly effective (failure rate<1%) medically accepted contraceptive method, including, but not limited to, bilateral tubal ligation/occlusion, hormone contraceptives that inhibit ovulation, intrauterine device (including hormone-releasing intrauterine device/systems) for 30 days before and 90 days after 5-MeO-DMT dosing and must have a negative serum pregnancy test at screening and a negative urine pregnancy test on the pre-test day (Day −1).
8. Male patients must use prophylactic contraception (i.e., condom with spermicide or abstinence) and must not donate sperm for 30 days after 5-MeO-DMT dosing.

A potential patient who meets any of the following key exclusion criteria will be excluded from participation in this trial:
1. Has, based on history, psychiatric assessment, and evaluation of the MINI, a current or prior diagnosis of bipolar I disorder, a manic episode, a psychotic disorder, major depressive disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigators judgment.
2. Has one or more first or second degree relatives with a current or previously diagnosed psychotic disorder, bipolar I disorder, or MDD with psychotic features.
3. Has significant suicide risk as defined by: (a) suicidal ideation as endorsed on items 4 or 5 on the C-SSRS within the past year, during the screening period, or at Baseline; or (b) suicidal behaviors within the past year; or (c) clinical assessment of significant suicidal risk during clinical interview; or (d) non-suicidal self-injury within the past year.
4. Has taken anti-depressive medication within 7 days or 5 half-lives (whichever is longer) prior to dosing (exception: within the last 5 weeks in the case of fluoxetine).
5. Has taken a medication with monoamine oxidase inhibitor (MAOI) activity within 14 days or 5 half-lives (whichever is longer) prior to dosing.
6. Has taken mood stabilizer therapy (e.g., lamotrigine, valproate, atypical antipsychotics) within 14 days (28 days for lithium) or 5 half-lives (whichever is longer) prior to dosing or takes mood stabilizer therapy at screening or is expected to require mood stabilizer therapy during the study (as per the investigators judgment).
7. Has previously experienced a significant adverse reaction to a hallucinogenic or psychedelic drug according to the investigator's judgment.
8. Has known allergies or hypersensitivity or any other contraindication to 5-MeO-DMT.
9. Has any current or past clinically significant condition (e.g., severe infection, severe pulmonary disease, •uncontrolled•hypertension, uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that may interfere with the interpretation of the trial results, constitutes a health risk for the patient, or that otherwise renders the patient unsuitable for the trial according to the investigator's judgment.
10. Takes any medication or other substance that renders the patient unsuitable for the trial according to the investigators judgment.
11. Has a clinically significant abnormality in physical examination, vital signs, electrocardiogram (ECG), or clinical laboratory parameters which renders the patient unsuitable for the trial according to the investigators judgment.
12. Female patient who has a positive pregnancy test at screening or on the pre-test day (Day −1), is pregnant or lactating, or plans to become pregnant during the course of the trial and up to 30 days after 5-MeO-DMT dosing.
13. Patients with DSM-5, alcohol or substance use disorder (excluding tobacco or caffeine use disorders) within 6 months prior to screening.

The primary objective of the trial is to determine the onset and durability of anti-depressive effects of a single-day individualized dosing regimen of 2 mg, 5 mg and 8 mg of 5-MeO-DMT in patients with bipolar II disorder and a current major episode of depression. Secondary Objectives are to determine the effect on depressive symptoms and global clinical status; the safety and tolerability; the intensity and duration of psychoactive effects (PsE); the impact on sleep quality; the impact on cognitive outcomes of a single-day individualized dosing regimen of 2 mg, 5 mg and 8 mg of 5-MeO-DMT in patients with bipolar II disorder and a current major episode of depression.

The primary endpoint of the study is the evaluation of the anti-depressive effects of 5-MeO-DMT by the change from baseline in MADRS assessed at Day 7.

Secondary endpoints include:
The anti-depressive effects of 5-MeO-DMT administered via intravenous injection evaluated by:
   The proportion of patients in remission (MADRS •10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (•50% reduction from baseline in MADRS total score) at 2 hours after the final study drug dosing on Day 0, and at Day 1 and Day 7;
   Change from baseline in CGI-S at 2 hours after the final study drug dosing on Day 0, and at Day 1 and Day 7.
   Change from baseline in BDRS at Day 1 and Day 7
The safety and tolerability of 5-MeO-DMT administered via intravenous injection evaluated by:
   Reporting of treatment-emergent adverse events (TEAEs);
   Clinically significant changes from baseline in ECG, vital signs, safety laboratory assessments and spirometry assessments;
   Assessment of sedation (Modified Observer's Assessment of Alertness and Sedation scale [MOAA/S]) following each dose (when the PsE have subsided and 60 minutes after each study drug dosing) and as part of the discharge evaluation on Day 0;
   The incidence of adverse events (AEs) of mania or hypomania (as assessed using the DSM-5 criteria for mania/hypomania);
   Change from baseline in YMRS assessed as part of the discharge evaluation on Day 0 and at Day 1 and Day 7;
   Change from baseline in Clinician Administered Dissociative States Scale (CADSS) assessed as part of the discharge evaluation on Day 0 and at Day 1 and Day 7;
   Assessment of patient discharge readiness at discharge on Day 0 using the Clinical Assessment of Discharge Readiness (CADR);
   Change from baseline in Brief Psychiatric Rating Scale (BPRS) assessed as part of the discharge evaluation on Day 0, and at Day 1 and Day 7;
   C-SSRS categorization based on the Columbia Classification Algorithm of Suicide Assessment (C-CASA).
The PsE experienced by the patients as reported 30 to 60 minutes after each dosing, when the PsE has subsided:
   PsE assessment using the peak experience (PE) Scale (PES) to assess the achievement of a PE (PES total score •75);
   Challenging Experience Questionnaire (CEQ);
   Mystical Experience Questionnaire (MEQ-30).
Duration of the PsE, defined as the time from study drug dosing to the time when the PsE have subsided, completed 30 to 60 minutes after each dosing.
The impact on sleep quality as evaluated by change from pre-test day (Day −1) to Day 1 and to Day 7 in the Pittsburgh Sleep Quality Index (PSQI).
The impact on cognitive outcomes as evaluated by change from the pre-test day (Day −1) to discharge on Day 0, to Day 1 and to Day 7 in:
   Rapid visual information processing (RVP) test;
   Verbal recognition memory (VRM) test;
   Spatial working memory (SWM) test;
   Digit symbol substitution test (DSST).

### Example 11—Clinical Trial of 5-MeO-DMT Administered Via Intravenous Injection to Patients with Bipolar II Disorder—Prophetic Example

The clinical trial will involve adult patients with bipolar II disorder and a current major depressive episode.

Patients who are currently taking anti-depressive medication need to discontinue or taper over time such medication.

The patients will receive a single-day individualized 5-MeO-DMT dosing regimen by intravenous injection. The 5-MeO-DMT will be provided in the form of its hydrobromide salt and a formulation for intravenous injection. It is understood that the dosage amounts for 5-MeO-DMT mentioned below relate to the weight amount of the free base and the dosage amounts for the hydrobromide salt of 5-MeO-DMT can be calculated assuming that equimolar amounts are used.

More in particular, the patients will receive up to three doses of 5-MeO-DMT on the administration day (Day 0): 1 mg, 2 mg, and 3 mg.
1. All patients will receive an initial dose of 1 mg 5-MeO-DMT.
2. The second dose (2 mg) will only be administered if:
   a. A peak experience (PES total score of •75) has not been achieved following the 1 mg dose, and
   b. The 1 mg dose was safe and well-tolerated.
3. Similarly, a third dose (3 mg) will only be administered if:
   a. A peak experience (PES total score of •75) has not been achieved following the 2 mg dose, and
   b. The 2 mg dose was safe and well-tolerated.

The patients will be assessed for a peak psychedelic experience based on the patient-scored PES described above, sedation and other endpoints after dosing. Follow-up visits are planned for Day 1, and Day 7 after the dosing day.

Selection of patients is based on the following key inclusion criteria:
1. Understands the nature of the clinical trial and has provided signed and dated written informed consent in accordance with local regulations before the conduct of any trial-related procedures.
2. Is male or female and in the age range between 18 and 64 years (inclusive) at screening.
3. Meets the trial criteria for bipolar II disorder and is experiencing a major depressive episode, as assessed by a trial psychiatrist or registered clinical psychologist:
   a. Meets the Diagnostic and Statistical Manual of Mental Disorders (DSM-5) diagnostic criteria for bipolar II disorder with a current major depressive disorder episode confirmed by the Mini-International Neuropsychiatric Interview (MINI);
   b. Has a Montgomery-Åsberg Depression Rating Scale (MADRS) total score of equal to or greater than 24 at screening and prior to first dose on Day 0;
4. Has a Young Mania Rating Scale (YMRS) total score less than or equal to 8 at screening and prior to first dose on Day 0;
5. Agrees to keep any psychotherapy unchanged, and not initiate any new psychoactive medications during the course of the trial.
6. Female patients must be either surgically sterile (hysterectomy, tubal ligation, or bilateral oophorectomy (6 months prior to screening)) or postmenopausal with amenorrhea for the last 2 years or remain completely abstinent (complete avoidance of heterosexual intercourse) or use a highly effective (failure rate<1%) medically accepted contraceptive method, including, but not limited to, bilateral tubal ligation/occlusion, hormone contraceptives that inhibit ovulation, intrauterine device (including hormone-releasing intrauterine device/systems) for 30 days before and 90 days after 5-MeO-DMT dosing and must have a negative serum pregnancy test at screening and a negative urine pregnancy test on the pre-test day (Day −1).
7. Male patients must use prophylactic contraception (i.e., condom with spermicide or abstinence) and must not donate sperm for 30 days after 5-MeO-DMT dosing.

A potential patient who meets any of the following key exclusion criteria will be excluded from participation in this trial:
1. Has, based on history, psychiatric assessment, and evaluation of the MINI, a current or prior diagnosis of bipolar I disorder, a manic episode, a psychotic disorder, major depressive disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigators judgment.
2. Has one or more first or second degree relatives with a current or previously diagnosed psychotic disorder, bipolar I disorder, or MDD with psychotic features.
3. Has significant suicide risk as defined by: (a) suicidal ideation as endorsed on items 4 or 5 on the C-SSRS within the past year, during the screening period, or at Baseline; or (b) suicidal behaviors within the past year; or (c)•clinical assessment of significant suicidal risk during clinical interview; or (d) non-suicidal self-injury within the past year.
4. Has taken anti-depressive medication within 7 days or 5 half-lives (whichever is longer) prior to dosing (exception: within the last 5 weeks in the case of fluoxetine).
5. Has taken a medication with monoamine oxidase inhibitor (MAOI) activity within 14 days or 5 half-lives (whichever is longer) prior to dosing.
6. Has taken mood stabilizer therapy (e.g., lamotrigine, valproate, atypical antipsychotics) within 14 days (28 days for lithium) or 5 half-lives (whichever is longer) prior to dosing or takes mood stabilizer therapy at screening or is expected to require mood stabilizer therapy during the study (as per the investigators judgment).
7. Has previously experienced a significant adverse reaction to a hallucinogenic or psychedelic drug according to the investigator's judgment.
8. Has known allergies or hypersensitivity or any other contraindication to 5-MeO-DMT.
9. Has any current or past clinically significant condition (e.g., severe infection, severe pulmonary disease, •uncontrolled•hypertension, uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that may interfere with the interpretation of the trial results, constitutes a health risk for the patient, or that otherwise renders the patient unsuitable for the trial according to the investigator's judgment.
10. Takes any medication or other substance that renders the patient unsuitable for the trial according to the investigators judgment.
11. Has a clinically significant abnormality in physical examination, vital signs, electrocardiogram (ECG), or clinical laboratory parameters which renders the patient unsuitable for the trial according to the investigators judgment.
12. Female patient who has a positive pregnancy test at screening or on the pre-test day (Day −1), is pregnant or lactating, or plans to become pregnant during the course of the trial and up to 30 days after 5-MeO-DMT dosing.
13. Patients with DSM-5, alcohol or substance use disorder (excluding tobacco or caffeine use disorders) within 6 months prior to screening.

The primary objective of the trial is to determine the onset and durability of anti-depressive effects of a single-day individualized dosing regimen of 1 mg, 2 mg and 3 mg of 5-MeO-DMT in patients with bipolar II disorder and a current major episode of depression. Secondary Objectives are to determine the effect on depressive symptoms and global clinical status; the safety and tolerability; the intensity and duration of psychoactive effects (PsE); the impact on sleep quality; the impact on cognitive outcomes of a single-day individualized dosing regimen of 1 mg, 2 mg and 3 mg of 5-MeO-DMT in patients with bipolar II disorder and a current major episode of depression.

The primary endpoint of the study is the evaluation of the anti-depressive effects of 5-MeO-DMT by the change from baseline in MADRS assessed at Day 7.

Secondary endpoints include:
The anti-depressive effects of 5-MeO-DMT administered via intravenous injection evaluated by:
   The proportion of patients in remission (MADRS •10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (•50% reduction from baseline in MADRS total score) at 2 hours after the final study drug dosing on Day 0, and at Day 1 and Day 7;
   Change from baseline in CGI-S at 2 hours after the final study drug dosing on Day 0, and at Day 1 and Day 7.
   Change from baseline in BDRS at Day 1 and Day 7
The safety and tolerability of 5-MeO-DMT administered via intravenous injection evaluated by:
   Reporting of treatment-emergent adverse events (TEAEs);
   Clinically significant changes from baseline in ECG, vital signs, safety laboratory assessments and spirometry assessments;
   Assessment of sedation (Modified Observer's Assessment of Alertness and Sedation scale [MOAA/S]) following each dose (when the PsE have subsided and 60 minutes after each study drug dosing) and as part of the discharge evaluation on Day 0;
   The incidence of adverse events (AEs) of mania or hypomania (as assessed using the DSM-5 criteria for mania/hypomania);
   Change from baseline in YMRS assessed as part of the discharge evaluation on Day 0 and at Day 1 and Day 7;
   Change from baseline in Clinician Administered Dissociative States Scale (CADSS) assessed as part of the discharge evaluation on Day 0 and at Day 1 and Day 7;
   Assessment of patient discharge readiness at discharge on Day 0 using the Clinical Assessment of Discharge Readiness (CADR);
   Change from baseline in Brief Psychiatric Rating Scale (BPRS) assessed as part of the discharge evaluation on Day 0, and at Day 1 and Day 7;
   C-SSRS categorization based on the Columbia Classification Algorithm of Suicide Assessment (C-CASA).
The PsE experienced by the patients as reported 30 to 60 minutes after each dosing, when the PsE has subsided:
   PsE assessment using the peak experience (PE) Scale (PES) to assess the achievement of a PE (PES total score •75);
   Challenging Experience Questionnaire (CEQ);
   Mystical Experience Questionnaire (MEQ-30).
Duration of the PsE, defined as the time from study drug dosing to the time when the PsE have subsided, completed 30 to 60 minutes after each dosing.
The impact on sleep quality as evaluated by change from pre-test day (Day −1) to Day 1 and to Day 7 in the Pittsburgh Sleep Quality Index (PSQI).
The impact on cognitive outcomes as evaluated by change from the pre-test day (Day −1) to discharge on Day 0, to Day 1 and to Day 7 in:
   Rapid visual information processing (RVP) test;
   Verbal recognition memory (VRM) test;
   Spatial working memory (SWM) test;
   Digit symbol substitution test (DSST).

1. 5-Methoxy-N,N-dimethyltryptamine (5-MeO-DMT) or a pharmaceutically acceptable salt thereof for use in treating a patient suffering from sleep disturbance, wherein the 5-MeO-DMT is administered via the intravenous, intramuscular or subcutaneous route.

2. 5-MeO-DMT or a pharmaceutically acceptable salt thereof for use as in claim 1, wherein the sleep disturbance is insomnia.

3. 5-MeO-DMT or a pharmaceutically acceptable salt thereof for use as in claim 1, wherein the sleep disturbance is hypersomnia.

4. 5-MeO-DMT or a pharmaceutically acceptable salt thereof for use as in claim 1, wherein the sleep disturbance is a circadian rhythm disorder.

5. 5-MeO-DMT or a pharmaceutically acceptable salt thereof for use as in claim 1, wherein the sleep disturbance is parasomnia.

6. 5-MeO-DMT or a pharmaceutically acceptable salt thereof for use as in claim 1, wherein the sleep disturbance is a sleep-related breathing disorder.

7. 5-MeO-DMT or a pharmaceutically acceptable salt thereof for use as in claim 1, wherein the sleep disturbance is a sleep-related movement disorder.

8. 5-MeO-DMT or a pharmaceutically acceptable salt thereof for use as in claims 1 to 7, wherein the sleep disturbance is an idiopathic sleep disturbance.

9. 5-MeO-DMT or a pharmaceutically acceptable salt thereof for use as in claim 1, wherein the patient is suffering from a mental or nervous system disorder associated with the sleep disturbance.

10. 5-MeO-DMT or a pharmaceutically acceptable salt thereof for use as in claim 9, wherein the patient suffering from a mental or nervous system disorder suffers from a treatment resistant form of the disorder.

11. 5-MeO-DMT or a pharmaceutically acceptable salt thereof for use as in claim 1, wherein the patient is suffering from a disorder characterized by depressive episodes associated with the sleep disturbance.

12. 5-MeO-DMT or a pharmaceutically acceptable salt thereof for use as in claim 11, wherein the patient suffering from a disorder characterized by depressive episodes suffers from a treatment resistant form of the disorder.

13. 5-MeO-DMT or a pharmaceutically acceptable salt thereof for use as in claim 1, wherein the patient is suffering from major depressive disorder (MDD) associated with the sleep disturbance.

14. 5-MeO-DMT or a pharmaceutically acceptable salt thereof for use as in claim 13, wherein the patient suffering from MDD suffers from a treatment resistant form of the disorder.

15. 5-MeO-DMT or a pharmaceutically acceptable salt thereof for use as in claim 1, wherein the patient is suffering from postpartum depression (PPD) associated with the sleep disturbance.

16. 5-MeO-DMT or a pharmaceutically acceptable salt thereof for use as in claim 15, wherein the patient suffering from PPD suffers from a treatment resistant form of the disorder.

17. 5-MeO-DMT or a pharmaceutically acceptable salt thereof for use as in claims 15 or 16, wherein the patient suffers in addition from compromised maternal functioning.

18. 5-MeO-DMT or a pharmaceutically acceptable salt thereof for use as in claim 17, wherein the patient has a Barkin Index of Maternal Functioning (BIMF) score of 80 or below, such as 65 or below.

19. 5-MeO-DMT or a pharmaceutically acceptable salt thereof for use as in claims 17 or 18, wherein the treatment improves maternal functioning.

20. 5-MeO-DMT or a pharmaceutically acceptable salt thereof for use as in claim 19, wherein the improvement in maternal functioning is reflected by an improvement of the BIMF total score by 10% or more, preferably by 20% or more.

21. 5-MeO-DMT or a pharmaceutically acceptable salt thereof for use as in claims 19 or 20, wherein the improvement in maternal functioning, is reflected by at least an improvement in the BIMF total score on day 7; on day 14; and/or on day 28 after the last administration of 5-MeO-DMT or a pharmaceutically acceptable salt thereof.

22. 5-MeO-DMT or a pharmaceutically acceptable salt thereof for use as in claims 19 or 20, wherein the improvement in maternal functioning, as reflected by at least an improvement in the BIMF total score, occurs not later than about 6 days after the last administration of 5-MeO-DMT or a pharmaceutically acceptable salt thereof and wherein the improvement in maternal functioning, as reflected by at least an improvement in the BIMF total score, persists until at least 14 days after the last administration of 5-MeO-DMT or a pharmaceutically acceptable salt thereof; more preferably until at least 28 days after the last administration of 5-MeO-DMT or a pharmaceutically acceptable salt thereof.

23. 5-MeO-DMT or a pharmaceutically acceptable salt thereof for use as in claim 1, wherein the patient is suffering from bipolar disorder associated with the sleep disturbance.

24. 5-MeO-DMT or a pharmaceutically acceptable salt thereof for use as in claim 23, wherein the patient is suffering from bipolar II disorder associated with the sleep disturbance.

25. 5-MeO-DMT or a pharmaceutically acceptable salt thereof for use as in claim 23, wherein the patient is suffering from bipolar I disorder associated with the sleep disturbance.

26. 5-MeO-DMT or a pharmaceutically acceptable salt thereof for use as in claims 23 to 25, wherein the patient suffers from a current major depressive episode.

27. 5-MeO-DMT or a pharmaceutically acceptable salt thereof for use as in claims 23 to 26, wherein the patient suffering from bipolar disorder suffers from a treatment resistant form of the disorder.

28. 5-MeO-DMT or a pharmaceutically acceptable salt thereof for use as in claim 1, wherein the patient is suffering from seasonal affective disorder associated with the sleep disturbance.

29. 5-MeO-DMT or a pharmaceutically acceptable salt thereof for use as in claim 28, wherein the patient suffering from seasonal affective disorder suffers from a treatment resistant form of the disorder.

30. 5-MeO-DMT or a pharmaceutically acceptable salt thereof for use as in claim 1, wherein the patient is suffering from persistent depressive disorder associated with the sleep disturbance.

31. 5-MeO-DMT or a pharmaceutically acceptable salt thereof for use as in claim 30, wherein the patient suffering from persistent depressive disorder suffers from a treatment resistant form of the disorder.

32. 5-MeO-DMT or a pharmaceutically acceptable salt thereof for use as in claim 1, wherein the patient is suffering from an anxiety disorder associated with the sleep disturbance.

33. 5-MeO-DMT or a pharmaceutically acceptable salt thereof for use as in claim 32, wherein the patient suffering from an anxiety disorder suffers from a treatment resistant form of the disorder.

34. 5-MeO-DMT or a pharmaceutically acceptable salt thereof for use as in claim 1, wherein the patient is suffering from separation anxiety disorder associated with the sleep disturbance.

35. 5-MeO-DMT or a pharmaceutically acceptable salt thereof for use as in claim 34, wherein the patient suffering from separation anxiety disorder suffers from a treatment resistant form of the disorder.

36. 5-MeO-DMT or a pharmaceutically acceptable salt thereof for use as in claim 1, wherein the patient is suffering from agoraphobia associated with the sleep disturbance.

37. 5-MeO-DMT or a pharmaceutically acceptable salt thereof for use as in claim 36, wherein the patient suffering from agoraphobia suffers from a treatment resistant form of the disorder.

38. 5-MeO-DMT or a pharmaceutically acceptable salt thereof for use as in claim 1, wherein the patient is suffering from generalised anxiety disorder (GAD) associated with the sleep disturbance.

39. 5-MeO-DMT or a pharmaceutically acceptable salt thereof for use as in claim 38, wherein the patient suffering from GAD suffers from a treatment resistant form of the disorder.

40. 5-MeO-DMT or a pharmaceutically acceptable salt thereof for use as in claim 1, wherein the patient is suffering from social anxiety disorder (SAD) associated with the sleep disturbance.

41. 5-MeO-DMT or a pharmaceutically acceptable salt thereof for use as in claim 40, wherein the patient suffering from SAD suffers from a treatment resistant form of the disorder.

42. 5-MeO-DMT or a pharmaceutically acceptable salt thereof for use as in claim 1, wherein the patient is suffering from panic disorder associated with the sleep disturbance.

43. 5-MeO-DMT or a pharmaceutically acceptable salt thereof for use as in claim 42, wherein the patient suffering from panic disorder suffers from a treatment resistant form of the disorder.

44. 5-MeO-DMT or a pharmaceutically acceptable salt thereof for use as in claim 1, wherein the patient is suffering from a phobia associated with the sleep disturbance.

45. 5-MeO-DMT or a pharmaceutically acceptable salt thereof for use as in claim 44, wherein the patient suffering from a phobia suffers from a treatment resistant form of the disorder.

46. 5-MeO-DMT or a pharmaceutically acceptable salt thereof for use as in claim 1, wherein the patient is suffering from substance/medication induced anxiety disorder associated with the sleep disturbance.

47. 5-MeO-DMT or a pharmaceutically acceptable salt thereof for use as in claim 46, wherein the patient suffering from substance/medication induced anxiety disorder suffers from a treatment resistant form of the disorder.

48. 5-MeO-DMT or a pharmaceutically acceptable salt thereof for use as in claim 1, wherein the patient is suffering from somatic symptom disorder associated with the sleep disturbance.

49. 5-MeO-DMT or a pharmaceutically acceptable salt thereof for use as in claim 48, wherein the patient suffering from somatic symptom disorder suffers from a treatment resistant form of the disorder.

50. 5-MeO-DMT or a pharmaceutically acceptable salt thereof for use as in claim 1, wherein the patient is suffering from an obsessive compulsive or related disorder associated with the sleep disturbance.

51. 5-MeO-DMT or a pharmaceutically acceptable salt thereof for use as in claim 50, wherein the obsessive compulsive or related disorder is obsessive compulsive disorder (OCD).

52. 5-MeO-DMT or a pharmaceutically acceptable salt thereof for use as in claims 50 or 51, wherein the patient suffers from a treatment resistant form of the disorder.

53. 5-MeO-DMT or a pharmaceutically acceptable salt thereof for use as in claim 1, wherein the patient is suffering from body dysmorphic disorder (BDD) associated with the sleep disturbance.

54. 5-MeO-DMT or a pharmaceutically acceptable salt thereof for use as in claim 53, wherein the patient suffering from BDD suffers from a treatment resistant form of the disorder.

55. 5-MeO-DMT or a pharmaceutically acceptable salt thereof for use as in claim 1, wherein the patient is suffering from post-traumatic stress disorder (PTSD) associated with the sleep disturbance.

56. 5-MeO-DMT or a pharmaceutically acceptable salt thereof for use as in claim 55, wherein the patient suffering from PTSD suffers from a treatment resistant form of the disorder.

57. 5-MeO-DMT or a pharmaceutically acceptable salt thereof for use as in claim 1, wherein the patient is suffering from a pain disorder associated with the sleep disturbance.

58. 5-MeO-DMT or a pharmaceutically acceptable salt thereof for use as in claim 57, wherein the patient is suffering from chronic pain associated with the sleep disturbance.

59. 5-MeO-DMT or a pharmaceutically acceptable salt thereof for use as in claims 57 or 58, wherein the patient suffers from a treatment resistant form of the disorder.

60. 5-MeO-DMT or a pharmaceutically acceptable salt thereof for use as in claim 1, wherein the patient is suffering from fibromyalgia associated with the sleep disturbance.

61. 5-MeO-DMT or a pharmaceutically acceptable salt thereof for use as in claim 1, wherein the patient is suffering from migraine associated with the sleep disturbance.

62. 5-MeO-DMT or a pharmaceutically acceptable salt thereof for use as in claim 1, wherein the patient is suffering from a mental and behavioural disorder due to psychoactive substance use associated with the sleep disturbance.

63. 5-MeO-DMT or a pharmaceutically acceptable salt thereof for use as in claim 62, wherein the patient is suffering from substance use disorder (SUD) associated with the sleep disturbance.

64. 5-MeO-DMT or a pharmaceutically acceptable salt thereof for use as in claims 62 or 63, wherein the patient suffers from a treatment resistant form of the disorder.

65. 5-MeO-DMT or a pharmaceutically acceptable salt thereof for use as in claim 1, wherein the patient is suffering from a psychotic disorder associated with the sleep disturbance.

66. 5-MeO-DMT or a pharmaceutically acceptable salt thereof for use as in claim 65, wherein the patient suffering from a psychotic disorder suffers from a treatment resistant form of the disorder.

67. 5-MeO-DMT or a pharmaceutically acceptable salt thereof for use as in claim 1, wherein the patient is suffering from schizophrenia associated with the sleep disturbance.

68. 5-MeO-DMT or a pharmaceutically acceptable salt thereof for use as in claim 67, wherein the patient suffering from schizophrenia suffers from a treatment resistant form of the disorder.

69. 5-MeO-DMT or a pharmaceutically acceptable salt thereof for use as in claim 1, wherein the patient is suffering from Huntington's disease associated with the sleep disturbance.

70. 5-MeO-DMT or a pharmaceutically acceptable salt thereof for use as in claim 1, wherein the patient is suffering from Parkinson's disease associated with the sleep disturbance.

71. 5-MeO-DMT or a pharmaceutically acceptable salt thereof for use as in claim 1, wherein the patient is suffering from dementia associated with the sleep disturbance.

72. 5-MeO-DMT or a pharmaceutically acceptable salt thereof for use as in claim 1, wherein the patient is suffering from Alzheimer's dementia associated with the sleep disturbance.

73. 5-MeO-DMT or a pharmaceutically acceptable salt thereof for use as in claim 1, wherein the patient is suffering from Parkinson's disease dementia associated with the sleep disturbance.

74. 5-MeO-DMT or a pharmaceutically acceptable salt thereof for use as in claim 1, wherein the patient is suffering from dementia with Lewy Bodies associated with the sleep disturbance.

75. 5-MeO-DMT or a pharmaceutically acceptable salt thereof for use as in claim 1, wherein the patient is suffering from vascular dementia associated with the sleep disturbance.

76. 5-MeO-DMT or a pharmaceutically acceptable salt thereof for use as in claim 1, wherein the patient is suffering from fronto-temporal dementia associated with the sleep disturbance.

77. 5-MeO-DMT or a pharmaceutically acceptable salt thereof for use as in claim 1, wherein the patient is suffering from an eating disorder associated with the sleep disturbance.

78. 5-MeO-DMT or a pharmaceutically acceptable salt thereof for use as in claim 77, wherein the patient suffering from an eating disorder suffers from a treatment resistant form of the disorder.

79. 5-MeO-DMT or a pharmaceutically acceptable salt thereof for use as in claim 1, wherein the patient is suffering from attention deficit hyperactivity disorder (ADHD) associated with the sleep disturbance.

80. 5-MeO-DMT or a pharmaceutically acceptable salt thereof for use as in claim 79, wherein the patient suffering from ADHD suffers from a treatment resistant form of the disorder.

81. 5-MeO-DMT or a pharmaceutically acceptable salt thereof for use as in claim 1, wherein the patient is suffering from a personality disorder associated with the sleep disturbance.

82. 5-MeO-DMT or a pharmaceutically acceptable salt thereof for use as in claim 81, wherein the patient is suffering from schizotypal personality disorder associated with the sleep disturbance.

83. 5-MeO-DMT or a pharmaceutically acceptable salt thereof for use as in claims 81 or 82, wherein the patient suffers from a treatment resistant form of the disorder.

84. 5-MeO-DMT or a pharmaceutically acceptable salt thereof for use as in claim 1, wherein the patient is suffering from a borderline personality disorder (BPD) associated with the sleep disturbance.

85. 5-MeO-DMT or a pharmaceutically acceptable salt thereof for use as in claim 84, wherein the patient suffering from an BPD suffers from a treatment resistant form of the disorder.

86. 5-MeO-DMT or a pharmaceutically acceptable salt thereof for use as in claim 1, wherein the patient is suffering from an autism spectrum disorder (ASD) associated with the sleep disturbance.

87. 5-MeO-DMT or a pharmaceutically acceptable salt thereof for use as in claim 1, wherein the patient is suffering from chronic fatigue syndrome associated with the sleep disturbance.

88. 5-MeO-DMT or a pharmaceutically acceptable salt thereof for use as in claim 87, wherein the patient suffering from chronic fatigue syndrome suffers from a treatment resistant form of the disorder.

89. 5-MeO-DMT or a pharmaceutically acceptable salt thereof for use as in claim 1, wherein the sleep disturbance is due to traumatic brain injury.

90. 5-MeO-DMT or a pharmaceutically acceptable salt thereof for use as in claim 1, wherein the sleep disturbance is due to HIV infection.

91. 5-MeO-DMT or a pharmaceutically acceptable salt thereof for use as in claim 1, wherein the sleep disturbance is due to post COVID condition.

92. 5-MeO-DMT or a pharmaceutically acceptable salt thereof for use as in claims 1 to 91, wherein the treatment reduces or eliminates the sleep disturbance.

93. 5-MeO-DMT or a pharmaceutically acceptable salt thereof for use as in claim 92, wherein the reduction or elimination of sleep disturbance is observed on day 1, for instance after about 24 hours; on day 7; on day 14; and/or on day 28 after the last administration of 5-MeO-DMT or a pharmaceutically acceptable salt thereof.

94. 5-MeO-DMT or a pharmaceutically acceptable salt thereof for use as in claim 92, wherein the reduction or elimination of sleep disturbance occurs not later than about 24 hours after the last administration of 5-MeO-DMT or a pharmaceutically acceptable salt thereof and wherein the reduction or elimination of sleep disturbance persists until at least 6 days after the last administration of 5-MeO-DMT or a pharmaceutically acceptable salt thereof; in particular until at least 14 days; more preferably until at least 28 days after the last administration of 5-MeO-DMT or a pharmaceutically acceptable salt thereof.

95. 5-MeO-DMT or a pharmaceutically acceptable salt thereof for use as in claim 92, wherein the reduction or elimination of sleep disturbance is observed on day 7 after the last administration of 5-MeO-DMT or a pharmaceutically acceptable salt thereof and preferably persists until at least 14 days, more preferably until at least 28 days.

96. 5-MeO-DMT or a pharmaceutically acceptable salt thereof for use as in claims 9 to 95, wherein the treatment leads to an improvement in the diagnosed disorder in a patient also suffering from associated sleep disturbance.

97. 5-MeO-DMT or a pharmaceutically acceptable salt thereof for use as in claim 96, wherein the improvement in the diagnosed disorder in a patient also suffering from associated sleep disturbance, as reflected by a reduction in the Clinical Global Impression-Severity (CGI-S) score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

98. 5-MeO-DMT or a pharmaceutically acceptable salt thereof for use as in claim 96, wherein the improvement in the diagnosed disorder in a patient also suffering from associated sleep disturbance, as reflected by a reduction in the Clinical Global Impression-Severity (CGI-S) score, occurs not later than about 2 hours after the last administration of 5-MeO-DMT or a pharmaceutically acceptable salt thereof and wherein the improvement in the diagnosed disorder in a patient also suffering from associated sleep disturbance, as reflected by a reduction in the Clinical Global Impression-Severity (CGI-S) score, persists until at least 6 days after the last administration of 5-MeO-DMT or a pharmaceutically acceptable salt thereof; in particular until at least 14 days; more preferably until at least 28 days after the last administration of 5-MeO-DMT or a pharmaceutically acceptable salt thereof.

99. 5-MeO-DMT or a pharmaceutically acceptable salt thereof for use as in claim 96, wherein the improvement in the diagnosed disorder in a patient also suffering from associated sleep disturbance, as reflected by a reduction in the Clinical Global Impression-Severity (CGI-S) score, is observed on day 7 after the last administration of 5-MeO-DMT or a pharmaceutically acceptable salt thereof and preferably persists until at least 14 days, more preferably until at least 28 days.

100. 5-MeO-DMT or a pharmaceutically acceptable salt thereof for use as in claims 1 to 99, wherein the 5-MeO-DMT or salt thereof is administered at a dose or in a dosage regimen that causes the patient to experience a peak psychedelic experience.

101. 5-MeO-DMT or a pharmaceutically acceptable salt thereof for use as in claims 1 to 100, wherein a dosage of about 1 mg to about 10 mg 5-MeO-DMT is administered, or wherein equimolar amounts of the pharmaceutically acceptable salt are administered instead of 5-MeO-DMT.

102. 5-MeO-DMT or a pharmaceutically acceptable salt thereof for use as in claims 1 to 100, wherein a dosage of about 2 mg; or of about 5 mg; or of about 8 mg is administered, or wherein equimolar amounts of the pharmaceutically acceptable salt are administered instead of 5-MeO-DMT.

103. 5-MeO-DMT or a pharmaceutically acceptable salt thereof for use as in claims 1 to 100, wherein a dosage of about 1 mg; or of about 2 mg; or of about 3 mg is administered, or wherein equimolar amounts of the pharmaceutically acceptable salt are administered instead of 5-MeO-DMT

104. 5-MeO-DMT or a pharmaceutically acceptable salt thereof for use as in claims 1 to 101, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.

105. 5-MeO-DMT or a pharmaceutically acceptable salt thereof for use as in claims 1 to 101 or 104, wherein the 5-MeO-DMT is administered in a dosage from about 1 mg to about 3 mg for a first administration, and then increased, unless the patient has already experienced a peak psychedelic experience, to a dosage from about 4 mg to about 6 mg for a second administration, and then increased, unless the patient has already experienced a peak psychedelic experience, to a dosage from about 7 mg to about 9 mg for a third administration, or wherein equimolar amounts of the pharmaceutically acceptable salt are administered instead of 5-MeO-DMT.

106. 5-MeO-DMT or a pharmaceutically acceptable salt thereof for use as in claim 105, wherein the first dosage of 5-MeO-DMT is about 2 mg, the second dosage of 5-MeO-DMT is about 5 mg, and the third dosage of 5-MeO-DMT is about 8 mg; or wherein equimolar amounts of the pharmaceutically acceptable salt are administered instead of 5-MeO-DMT.

107. 5-MeO-DMT or a pharmaceutically acceptable salt thereof for use as in claims 1 to 101 or 104, wherein the 5-MeO-DMT is administered in a dosage from about 0.5 mg to about 1.5 mg for a first administration, and then increased, unless the patient has already experienced a peak psychedelic experience, to a dosage from about 1.5 mg to about 2.5 mg for a second administration, and then increased, unless the patient has already experienced a peak psychedelic experience, to a dosage from about 2.5 mg to about 3.5 mg for a third administration, or wherein equimolar amounts of the pharmaceutically acceptable salt are administered instead of 5-MeO-DMT.

108. 5-MeO-DMT or a pharmaceutically acceptable salt thereof for use as in claim 107, wherein the first dosage of 5-MeO-DMT is about 1 mg, the second dosage of 5-MeO-DMT is about 2 mg, and the third dosage of 5-MeO-DMT is about 3 mg; or wherein equimolar amounts of the pharmaceutically acceptable salt are administered instead of 5-MeO-DMT.

109. 5-MeO-DMT or a pharmaceutically acceptable salt thereof for use as in claims 104 to 108, wherein the interval between two administrations is not less than 1 hour and not more than 24 hours, such as about 1 to 4 hours, preferably 1 to 2 hours.

110. 5-MeO-DMT or a pharmaceutically acceptable salt thereof for use as in claims 100 to 109, wherein the occurrence of a peak psychedelic experience is identified through achievement of at least 60% of the maximum possible score in each of the four subscales (mystical, positive mood, transcendence of time and space, and ineffability) of the 30-item revised Mystical Experience Questionnaire (MEQ30) or is identified through achievement of at least 60% of the maximum possible score of the Oceanic Boundlessness (OBN) dimension of the Altered States of Consciousness (ASC) questionnaire or is identified through achievement of a Peak Experience Scale (PES) Total Score of at least 75.

111. 5-MeO-DMT or a pharmaceutically acceptable salt thereof for use as in claim 110, wherein the occurrence of a peak psychedelic experience is identified through achievement of a Peak Experience Scale (PES) Total Score of at least 75.

112. 5-MeO-DMT or a pharmaceutically acceptable salt thereof for use as in claims 1 to 111, wherein the 5-MeO-DMT or a pharmaceutically acceptable salt thereof is administered via intravenous injection.
